(19) United States
(12) Patent Application Publication
(10) Pub. No.: US 2025/0380945 A1
(43) Pub. Date: Dec. 18, 2025

(54) RELOAD ASSEMBLY OF SURGICAL INSTRUMENT

(71) Applicant: LIVSMED INC., Seongnam-si (KR)

(72) Inventor: Woo Jung CHOI, Siheung-si (KR)

(21) Appl. No.: 19/237,974

(22) Filed: Jun. 13, 2025

(30) Foreign Application Priority Data
Jun. 18, 2024 (KR) 10-2024-0078791

(51) Int. Cl. A61B 17/072; A61B 17/00

(52) U.S. Cl. CPC A61B 17/072; A61B 2017/00323; A61B 2017/00398; A61B 2017/07278

(57) ABSTRACT

A reload assembly of a surgical instrument includes an end tool including one or more rotatable jaws and configured to be rotated in at least one direction, a shaft having an end to which the end tool is connected, and a power transmission part connected to the shaft and configured to transmit power generated by a power generation part to the end tool. The power transmission part includes a pulley frame connected to the power generation part, a driving pulley unit accommodated in the pulley frame and configured to receive the power generated by the power generation part and perform an axial rotation, and a wire unit having one end portion connected to the driving pulley unit and another end portion connected to the end tool and configured to transmit the power generated by the power generation part to the end tool.

### CROSS-REFERENCE TO RELATED APPLICATION

This application is based on and claims priority under 35 U.S.C. § 119 to Korean Patent Application No. 10-2024-0078791, filed on Jun. 18, 2024, in the Korean Intellectual Property Office, the disclosure of which is incorporated by reference herein in its entirety.

### BACKGROUND

### 1. Field

The disclosure relates to a surgical instrument, and more particularly, to a surgical instrument mounted on a robot arm or manually operable for use in laparoscopic surgery or various other surgeries.

### 2. Description of the Related Art

In medical terms, surgery refers to curing an illness by cutting, incising, or manipulating the skin, mucous membranes, or other tissues by using medical devices. In particular, open surgery, which involves cutting open the skin at the surgical site and treating, shaping, or removing the organs inside, causes problems such as bleeding, side effects, patient pain, and scarring. Therefore, surgery using a robot or surgery performed by forming a certain hole in the skin and inserting only a medical device, such as a laparoscope, a surgical instrument, or a microsurgical microscope, has recently attracted attention as an alternative.

A surgical instrument is a tool for operating on a surgical site by manipulating an end tool provided at an end of a shaft which passes through a hole drilled in the skin. A medical doctor may manipulate the end tool with his/her hand by using a certain driver, or may manipulate the end tool by using a robot arm. The end tool provided in the surgical instrument performs a rotating motion, a gripping motion, a cutting motion, or the like through a certain structure.

The aforementioned background technology is technical information possessed by the inventor for derivation of the disclosure or acquired by the inventor during the derivation of the disclosure, and is not necessarily prior art disclosed to the public before the application of the disclosure.

### SUMMARY

The present disclosure provides a surgical instrument which may be mounted on a robot arm or manually operated for use in laparoscopic surgery or various other surgeries, wherein the surgical instrument is capable of an axial rotation (roll) without any limitation of rotation angle.

In an embodiment, a reload assembly of a surgical instrument includes an end tool including one or more rotatable jaws and configured to be rotated in at least one direction, a shaft having an end to which the end tool is connected, and a power transmission part connected to the shaft and configured to transmit power generated by a power generation part to the end tool, wherein the power transmission part includes a pulley frame connected to the power generation part, a driving pulley unit accommodated in the pulley frame and configured to receive the power generated by the power generation part and perform an axial rotation, and a wire unit having one end portion connected to the driving pulley unit and another end portion connected to the end tool and configured to transmit the power generated by the power generation part to the end tool. A rotation shaft of the driving pulley unit may be arranged in a direction extending toward the end tool.

In another embodiment, the driving pulley unit may include a first driving pulley, the wire unit may include a first wire wound around the first driving pulley in a first direction and a second wire wound around the first driving pulley in a second direction opposite to the first direction, and by unidirectional rotation of the first driving pulley, one of the first wire and the second wire may be wound around the first driving pulley and another of the first wire and the second wire may be unwound from the first driving pulley.

In the other embodiment, a groove having a shape of a thread root may be formed on an outer circumferential surface of the first driving pulley, and the first wire and the second wire may be wound around the groove.

In an embodiment, a portion of the first wire fixed to the first driving pulley may be constant, and a portion of the second wire fixed to the first driving pulley may be variable.

In the other embodiment, the groove may be formed continuously to allow the first wire and the second wire to be wound around the groove, and when one of the first wire and the second wire is unwound from a wound position as the first driving pulley is rotated, another of the first wire and the second wire may be wound at the wound position.

In the other embodiment, the driving pulley unit may include a pair of driving pulleys arranged symmetrically with respect to a central axis of the shaft.

In an embodiment, the driving pulley unit may include at least one firing driving pulley around which a forward wire and a backward wire are wound, a yaw driving pulley around which a yaw wire is wound, and a pitch driving pulley around which a pitch wire is wound.

In the other embodiment, the driving pulley unit may include a pair of firing driving pulleys, and the pair of firing driving pulleys may be arranged symmetrically with respect to a center shaft of the shaft.

In the other embodiment, the power transmission part may further include an auxiliary pulley unit contacting at least a portion of the wire unit and configured to change a progressing path of the wire unit extending from the driving pulley unit and guide the wire unit into the shaft.

In the other embodiment, the driving pulley unit may include a first driving pulley and a second driving pulley arranged symmetrically with respect to the central axis of the shaft, and the auxiliary pulley unit may include a center auxiliary pulley arranged between the first driving pulley and the second driving pulley.

In the other embodiment, the center auxiliary pulley may be axially coupled to a center auxiliary pulley rotation shaft, and the center auxiliary pulley rotation shaft may be arranged to cross an imaginary plane passing through the first driving pulley and the second driving pulley.

In the other embodiment, the center auxiliary pulley rotation shaft may be configured to receive forces from a plurality of wires passing through the center auxiliary pulley, and a portion of the forces applied in a direction perpendicular to the center auxiliary pulley rotation shaft may be partially offset from each other.

In the other embodiment, the first driving pulley and the second driving pulley may manipulate a same degree of freedom of the end tool.

In the other embodiment, the center auxiliary pulley may include a first auxiliary pulley, a second auxiliary pulley, a third auxiliary pulley, and a fourth auxiliary pulley, which are all axially coupled about a same rotation shaft, the first auxiliary pulley and the second auxiliary pulley may be arranged on one side with respect to the central axis of the shaft, and the third auxiliary pulley and the fourth auxiliary pulley may be arranged on another side with respect to the central axis of the shaft.

In the other embodiment, the wire unit may include a pair of wires including a forward wire and a backward wire, one of the forward wire and the backward wire may pass through one of the first auxiliary pulley and the second auxiliary pulley, and another of the forward wire and the backward wire may pass through one of the third auxiliary pulley and the fourth auxiliary pulley.

In the other embodiment, a first forward wire and a first backward wire may be wound around the first driving pulley, a second forward wire and a second backward wire may be wound around the second driving pulley, the first forward wire may be wound around one of the first auxiliary pulley and the second auxiliary pulley, the second backward wire may be wound around another of the first auxiliary pulley and the second auxiliary pulley, the second forward wire may be wound around one of the third auxiliary pulley and the fourth auxiliary pulley, and the first backward wire may be wound around another of the third auxiliary pulley and the fourth auxiliary pulley.

In the other embodiment, the driving pulley unit may include a first driving pulley, the auxiliary pulley unit may include at least one pair of auxiliary pulleys, and each of the at least one pair of auxiliary pulleys may be spaced apart from each other corresponding to a diameter of the first driving pulley around which a pair of wires are wound.

In the other embodiment, the pulley frame may be configured to accommodate the driving pulley unit to allow the driving pulley unit to be axially rotatable.

In the other embodiment, the pulley frame may include a first pulley frame including an auxiliary pulley fixing part configured to accommodate at least a portion of auxiliary pulley unit, and a second pulley frame configured to connect the shaft to the first pulley frame.

In the other embodiment, one end portion of the driving pulley unit may be coupled to the first pulley frame, and another end portion opposite to the end portion may be coupled to the second pulley frame.

In the other embodiment, the auxiliary pulley unit may include a center auxiliary pulley arranged adjacent to a central axis of the shaft, and the auxiliary pulley fixing part may include a center auxiliary pulley fixing part extending from the first pulley frame toward the shaft and configured to accommodate the center auxiliary pulley.

In the other embodiment, a surgical instrument includes an end tool including one or more rotatable jaws and configured to be rotated in at least one direction, a shaft having an end to which the end tool is connected, a power generation part configured to generate power for driving the end tool, and a power transmission part arranged between the shaft and the power generation part and configured to transmit the power generated by a power generation part to the end tool, wherein the power transmission part includes a pulley frame connected to the power generation part, a driving pulley unit accommodated in the pulley frame and configured to receive the power generated by the power generation part and perform an axial rotation, and a wire unit having one end portion connected to the driving pulley unit and another end portion connected to the end tool and configured to transmit the power generated by the power generation part to the end tool, wherein a rotation shaft of the driving pulley unit is arranged in a direction extending toward the end tool.

In the other embodiment, the surgical instrument may further include a manipulation part configured to receive, from a user, a signal for controlling an operation of the end tool, wherein the power generation part may include a motor pack including at least one motor arranged to be at least partially accommodated in a housing of the manipulation part and configured to generate power for driving the end tool, based on a signal input to the manipulation part, and a roll driving motor configured to generate power for a roll rotation of the motor pack.

In the other embodiment, the power transmission part may be connected to the motor pack and configured to be roll-rotated together with the motor pack.

In an embodiment, the end tool and the shaft may be roll-rotated together by the roll rotation of the power transmission part.

In the other embodiment, the end tool may further include a moving member configured to move along a longitudinal direction of the end tool, and the motor pack may further include a yaw driving motor configured to generate power for a yaw rotation of the end tool, a pitch driving motor configured to generate power for a pitch rotation of the end tool, and a firing driving motor configured to generate power for a linear motion of the moving member.

In the other embodiment, the roll driving motor, the yaw driving motor, the pitch driving motor, and the firing driving motor may be driven independently of each other to independently perform the yaw rotation of the end tool, the pitch rotation of the end tool, the roll rotation of the motor pack, and the linear motion of the movable member.

In the other embodiment, the driving pulley unit may include a yaw driving pulley connected to the yaw driving motor, a pitch driving pulley connected to the pitch driving motor, and a firing driving pulley connected to the firing driving motor.

In the other embodiment, the power generation part may further include a base plate connected to the roll driving motor, the yaw driving motor, the pitch driving motor, and the firing driving motor, wherein the base plate may be rotated by the driving of the roll driving motor, and the roll driving motor, the yaw driving motor, the pitch driving motor, and the firing driving motor may be rotated simultaneously by the rotation of the base plate.

Other aspects, features, and advantages of the disclosure will become better understood through the accompanying drawings, the appended claims, and the detailed description.

### BRIEF DESCRIPTION OF THE DRAWINGS

The above and other aspects, features, and advantages of certain embodiments of the disclosure will be more apparent from the following description taken in conjunction with the accompanying drawings.

FIG. 1 is a perspective view illustrating a surgical instrument according to a first embodiment.

FIG. 2 is a perspective view illustrating the remaining parts of the surgical instrument according to the first embodiment, excluding a manipulation part.

FIG. 3 is a schematic perspective view for describing an end tool of FIG. 1.

FIG. 4 is a perspective view of the end tool of FIG. 3, when viewed from another direction.

FIG. 5 is a schematic perspective view of the end tool of FIG. 3, from which a second jaw is removed.

FIG. 6 is a schematic perspective view of the end tool of FIG. 5, from which a cartridge is removed.

FIG. 7 is a phantom perspective view of FIG. 6.

FIG. 8 is a perspective view illustrating a first jaw and a cartridge of the surgical instrument of FIG. 1.

FIG. 9 is a perspective view illustrating a power transmission part and a shaft of FIG. 2.

FIG. 10 is a diagram for describing the internal structure of the power transmission part according to the first embodiment.

FIG. 11 is a diagram illustrating the power transmission part of FIG. 10, when viewed from behind.

FIG. 12 is a perspective view illustrating a first pulley frame in the power transmission part of FIG. 10.

FIG. 13 is a perspective view illustrating a second pulley frame in the power transmission part of FIG. 9.

FIG. 14 is a cross-sectional perspective view illustrating the side cross-section of the second pulley frame of FIG. 13.

FIG. 15 is a side view illustrating a driving pulley of the power transmission part according to the first embodiment.

FIG. 16 is an exploded perspective view of the driving pulley of FIG. 15.

FIGS. 17A and 17B are diagrams for describing a relationship between a driving pulley and a wire according to a driving process of the power transmission part according to the first embodiment.

FIGS. 18 to 20 are diagrams for describing a process in which a pair of wires are wound around or unwound from the driving pulley of FIG. 15.

FIG. 21 is a perspective view illustrating a state where the first pulley frame of the power transmission part of FIG. 10 is removed.

FIG. 22 is a diagram illustrating the power transmission part of FIG. 21, when viewed from the front in an X-axis direction.

FIG. 23 is a perspective view illustrating wires connected to a driving pulley and a center auxiliary pulley in the power transmission part of FIG. 21.

FIG. 24 is a diagram for describing an auxiliary pulley arranged on the first pulley frame in the power transmission part of FIG. 10.

FIG. 25 is a perspective view illustrating a state where a wire is wound around the auxiliary pulley of FIG. 24.

FIG. 26 is a diagram for describing an internal structure of a power transmission part according to a second embodiment.

FIG. 27 is a diagram illustrating the power transmission part of FIG. 26, when viewed from behind.

FIG. 28 is a diagram for describing an arrangement of a pulley and a wire in the power transmission part of FIG. 26.

FIG. 29 is a perspective view illustrating a center auxiliary pulley and a wire in the power transmission part of FIG. 28.

FIG. 30 is a diagram illustrating the center auxiliary pulley and the wire of FIG. 29, when viewed from the front.

FIG. 31 is a perspective view illustrating the auxiliary pulley and the wire in the power transmission part of FIG. 28.

FIGS. 32 and 33 are side views illustrating the auxiliary pulley and the wire of FIG. 31, when viewed from a side.

FIG. 34 is a perspective view illustrating the arrangement of the auxiliary pulley and the wire in the power transmission part of FIG. 28, excluding the driving pulley.

FIG. 35 is a diagram illustrating the auxiliary pulley and the wire of FIG. 34, when viewed from the front.

FIGS. 36 and 37 are diagrams for describing a process in which a pair of wires are wound around or unwound from the driving pulley of FIG. 26.

FIG. 38 is a diagram for describing an internal structure of a power transmission part according to a third embodiment.

FIG. 39 is a diagram illustrating the power transmission part according to the third embodiment, when viewed from behind.

FIG. 40 is a diagram for describing an arrangement of a pulley and a wire in the power transmission part of FIG. 38.

FIG. 41 is a perspective view illustrating an auxiliary pulley and a wire in the power transmission part of FIG. 40.

FIG. 42 is a diagram illustrating the auxiliary pulley and the wire of FIG. 41, when viewed from the front.

FIG. 43 is a diagram illustrating a manipulation part and a power generation part of a surgical instrument according to a second embodiment.

FIG. 44 is a perspective view illustrating the power generation part of FIG. 43.

FIG. 45 is a diagram illustrating the power generation part of FIG. 44, when viewed from behind.

FIG. 46 is a diagram for describing a gear structure of the power generation part of FIG. 44.

FIG. 47 is a diagram illustrating the gear structure of FIG. 46, when viewed from the front.

FIG. 48 is a diagram for describing the rotation of the power generation part of FIG. 44.

FIG. 49 is a diagram for describing a roll motion of the surgical instrument according to the second embodiment.

FIG. 50 is a diagram for describing a coupling structure of the surgical instrument according to the second embodiment.

FIG. 51 is a diagram illustrating the internal structure of the surgical instrument according to the second embodiment.

FIG. 52 is a diagram for describing a coupling structure of the surgical instrument according to the first embodiment.

FIGS. 53 to 57 are diagrams illustrating a pitch rotation motion of a surgical instrument according to an embodiment.

FIGS. 58 to 62 are diagrams illustrating a yaw rotation motion of a surgical instrument according to an embodiment.

FIGS. 63 to 67 are diagrams illustrating a state where a surgical instrument according to an embodiment performs a pitch rotation motion and a yaw rotation motion.

### DETAILED DESCRIPTION

Hereinafter, the following embodiments will be described in detail with reference to the accompanying drawings. When describing the embodiments with reference to the accompanying drawings, the same or corresponding elements are denoted by the same reference numerals and redundant descriptions thereof are omitted.

Since various changes may be made to the present embodiments, specific embodiments will be illustrated in the drawings and described in detail in the detailed description. Effects and features of the present embodiments, and methods of achieving them will be clarified with reference to the detailed description below along with the drawings. However, the present embodiments are not limited to the embodiments disclosed below and may be implemented in various forms.

In describing the disclosure, when the detailed description of the relevant known technology is determined to obscure the gist of the disclosure, the detailed description thereof may be omitted.

The singular forms as used herein are intended to include the plural forms as well unless the context clearly indicates otherwise. While the terms such as “first” and “second” may be used to describe various elements, the elements should not be limited by the terms. These terms are only used to distinguish one element from another.

In the following embodiments, it will be understood that the terms “include” and/or “comprise” used herein specify the presence of stated features or elements, but do not preclude the presence or addition of one or more other features or elements.

In the following embodiments, it will be understood that, when a portion such as unit, region, or element is referred to as being “on” another portion, this may include not only a case where the portion is directly on the other portion, but also a case where intervening units, regions, or elements may be present therebetween.

In the following embodiments, it will be understood that the terms “connection” or “coupling” do not necessarily mean “direct and/or fixed connection or coupling” of two members, unless the context clearly indicates otherwise, and this does not preclude the disposition of other members between the two members.

Also, sizes of elements in the drawings may be exaggerated or reduced for convenience of explanation. For example, since sizes and thicknesses of elements in the drawings are arbitrarily illustrated for convenience of explanation, the following embodiments are not limited thereto.

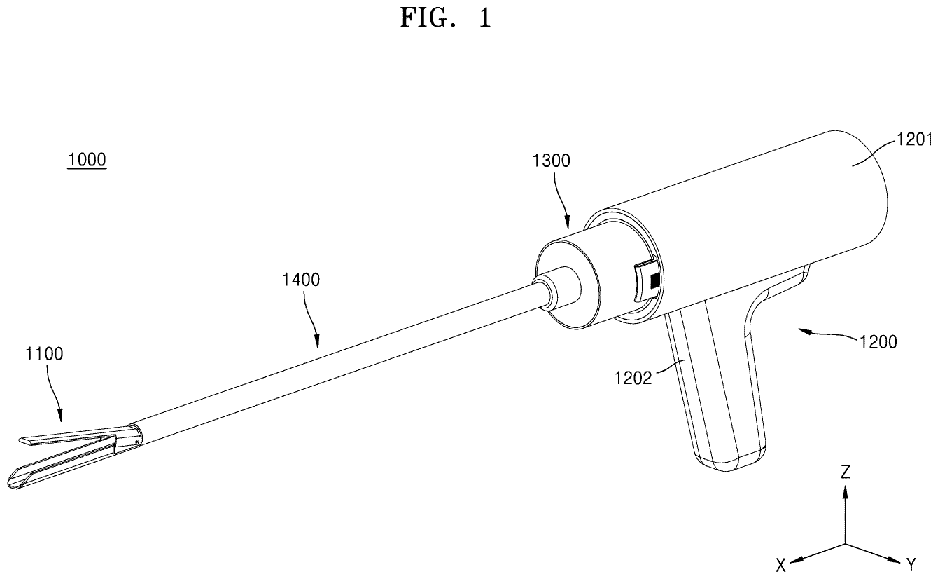

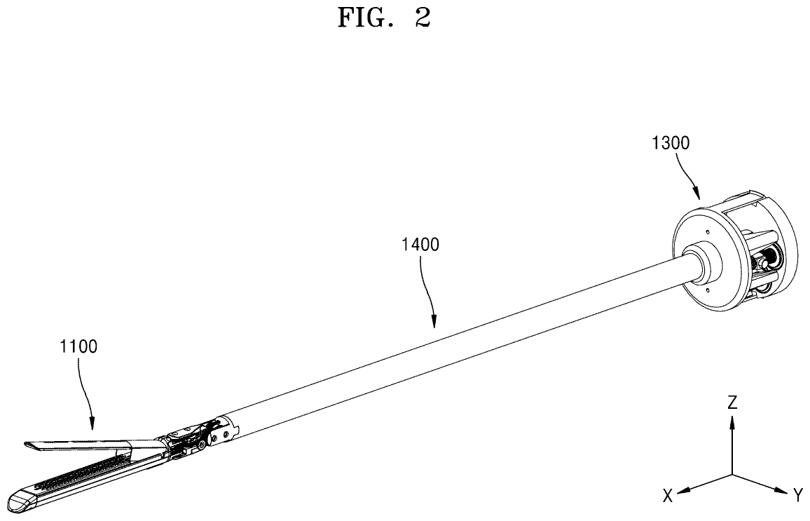

FIG. 1 is a perspective view illustrating a surgical instrument according to a first embodiment, and FIG. 2 is a perspective view illustrating the remaining portions of the surgical instrument according to the first embodiment, excluding the manipulation part.

Referring to FIGS. 1 and 2, a surgical instrument 1000 according to the present embodiment may include an end tool 1100, a manipulation part 1200, a connection part (or a shaft) 1400, and a power transmission part 1300.

In some embodiments, as illustrated in FIG. 2, the configuration including the end tool 1100, the connection part 1400, and the power transmission part 1300 is referred to as a reload assembly.

The connection part 1400 may be formed in the shape of a hollow shaft, and one or more wires and electric wires may be accommodated in the connection part 1400. For convenience of explanation of the present embodiment, the shaft is referred to as the connection part 1400. The manipulation part 1200 may be coupled to one end portion of the connection part 1400 and the end tool 1100 may be coupled to another end portion of the connection part 1400, such that the connection part 1400 may serve to connect the manipulation part 1200 to the end tool 1100. For example, the connection part 1400 may include a straight portion 1401. Although not illustrated, the connection part 1400 may include one or more curved portions for ease of use and arrangement control of manipulation configuration.

The power transmission part 1300 may be formed at the other end portion of the connection part 1400 and may transmit, to the end tool 1100, power generated by a power generation part described below. For example, the power transmission part 1300 may be arranged between the end tool 1100 and the manipulation part 1200. As described below, when a user, such as a medical doctor, manipulates the manipulation part 1200, the power generation part may generate power for controlling the end tool 1100, and the generated power may be transmitted to the end tool 1100 through the power transmission part 1300. The power transmission part 1300 may include a plurality of wires, pulleys, links, joints, gears, or the like. The power transmission part 1300 is described in detail below with reference to embodiments.

The user may operate the end tool 1100 by manipulating the manipulation part 1200. For example, the manipulation part 1200 may be configured to allow the user to input a signal for controlling the operation of the end tool 1100. The manipulation part 1200 may also be configured to receive, from the user, the signal for controlling the operation of the end tool 1100. The signal for controlling the operation of the end tool 1100 may be a mechanical manipulation, such as a pressing motion of a button or a switch, a mechanical manipulation, such as rotation or movement of a specific member, or an electrical signal generated by the mechanical manipulation, but the disclosure is not limited thereto. The manipulation part 1200 is provided as an interface to be directly controlled by a medical doctor, for example, a gun shape, a tongs shape, a stick shape, a lever shape, or the like, and when the medical doctor controls the manipulation part 1200, the end tool 1100, which is connected to the interface and inserted into the body of a surgical patient, performs a certain motion, thereby performing surgery. Although FIG. 1 illustrates that the manipulation part 1200 is formed in a gun shape, the concept of the disclosure is not limited thereto, and various types of manipulation parts which may be connected to the end tool 1100 and manipulate the end tool 1100 are possible.

The manipulation part 1200 may include a housing 1201 which forms the outer shape of the manipulation part 1200. As described below, at least a portion of the power generation part which generates power for controlling the end tool 1100 may be accommodated in the housing 1201. In some embodiments, a circuit unit which controls the operation of the power generation part and a slip ring which supplies electrical energy to the power generation part, connects communication, or transmits various other signals may be accommodated in the housing 1201.

A handle 1202 may be formed in the manipulation part 1200. The handle 1202 may be a portion held by the user. Accordingly, the user may use the surgical instrument 1000 according to the disclosure while holding the handle 1202 of the manipulation part 1200.

In some embodiments, although not illustrated, a button, a switch, a lever, or the like may be further formed on the manipulation part 1200 to control various operations of the end tool 1100.

The end tool 1100 may be formed at the other end portion of the connection part 1400 and may be inserted into a surgical site to perform motions necessary for surgery. As an example of the end tool 1100 described above, a pair of jaws 1103 may be used to perform a gripping motion. However, the concept of the disclosure is not limited thereto, and various devices for performing surgery may be used as the end tool 1100. For example, a configuration of a cantilever cautery may also be used as the end tool 1100. The end tool 1100 may be connected to the manipulation part 1200 by the power transmission part 1300 (for example, a wire or the like) and receive a driving force of the manipulation part 1200 through the power transmission part 1300 to perform a motion necessary for surgery, such as gripping, cutting, suturing, or the like. Hereinafter, the end tool 1100 of the surgical instrument 1000 of FIG. 1 is described in more detail.

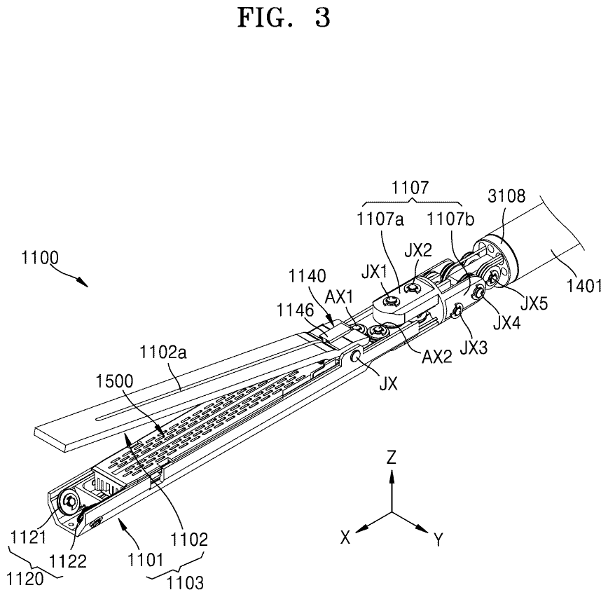

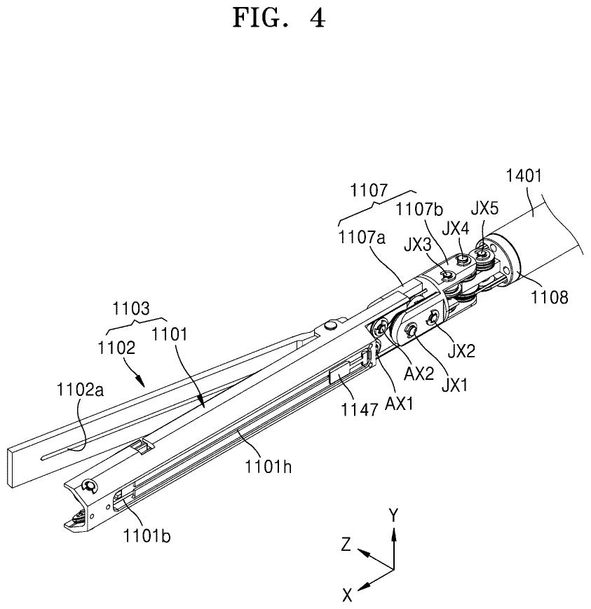

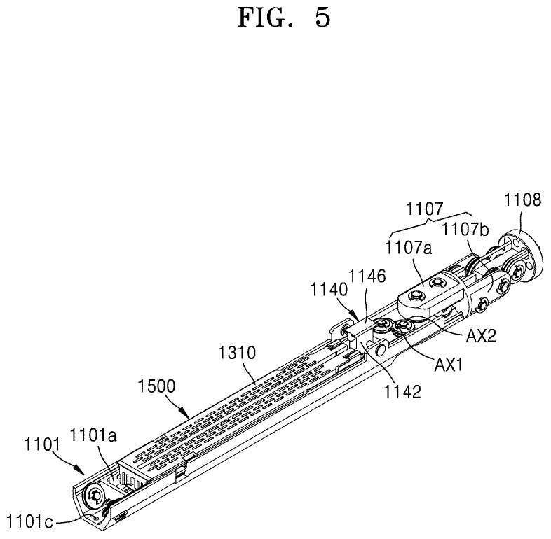

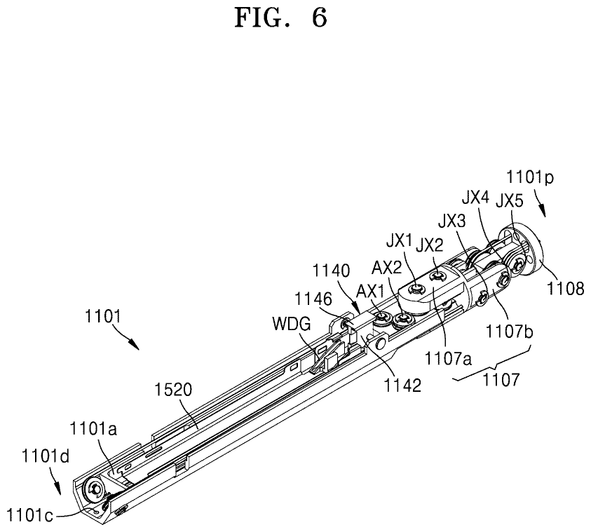

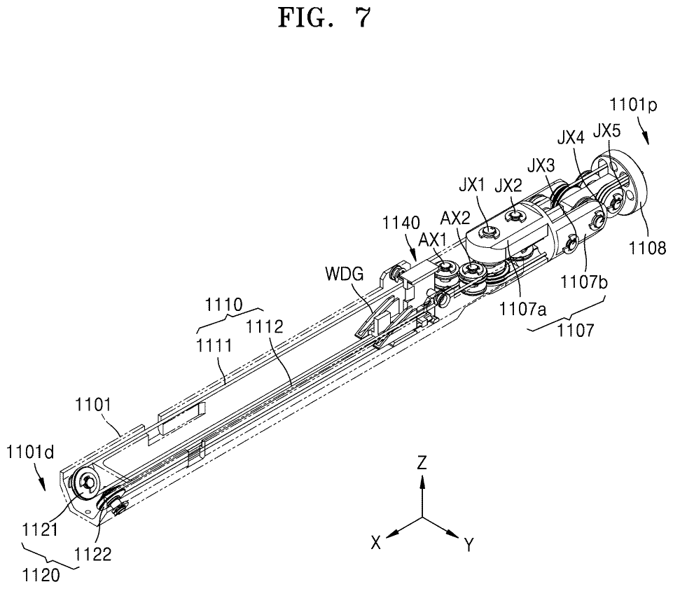

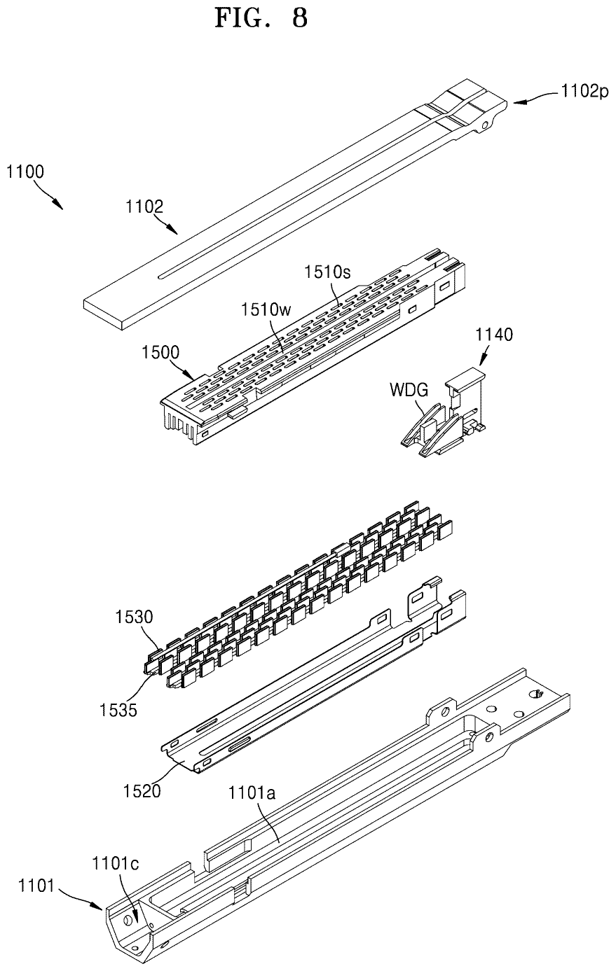

FIG. 3 is a schematic perspective view for describing the end tool of FIG. 1, and FIG. 4 is a perspective view of the end tool of FIG. 3, when viewed from another direction. FIG. 5 is a schematic perspective view of the end tool of FIG. 3 from which a second jaw is removed, FIG. 6 is a schematic perspective view of the end tool of FIG. 5 from which a cartridge is removed, and FIG. 7 is a phantom perspective view of FIG. 6. FIG. 8 is a perspective view illustrating a first jaw and a cartridge of the surgical instrument of FIG. 1.

The end tool 1100 may include a jaw 1103, a plurality of fixed pulleys 1120, and a plurality of forward wires 1110. The plurality of fixed pulleys 1120 may include two or more pulleys, for example, a first fixed pulley 1121 and a second fixed pulley 1122. The plurality of forward wires 1110 may include two or more wires, for example, a first forward wire 1111 and a second forward wire 1112.

The jaw 1103 may perform various functions, for example, a gripping motion. As a specific example, the jaw 1103 may include a pair of jaws, that is, a first jaw 1101 and a second jaw 1102. Each of the first jaw 1101 and the second jaw 1102 or an element encompassing the first jaw 1101 and the second jaw 1102 may be referred to as the jaw 1103.

The first jaw 1101 and the second jaw 1102 may be arranged to face each other and may move closer to each other and move away from each other. For example, the first jaw 1101 and the second jaw 1102 may be formed to rotate around a shaft JX.

A cartridge 1500 may be arranged to be accommodated in the first jaw 1101, and a plurality of staples may be arranged inside the cartridge 1500. When an operation member 1140 receives a force from the plurality of forward wires 1110 in a state where the first jaw 1101 and the second jaw 1102 are close to each other, for example, in a state where body tissue is arranged between the first jaw 1101 and the second jaw 1102 so that the first jaw 1101 and the second jaw 1102 are closed, the operation member 1140 may move in a direction of a distal end 1101d of the first jaw 1101 and push and raise the staples so that stapling is performed. At this time, while applying pressure to the outer surfaces of the first jaw 1101 and the second jaw 1102 in a state of protruding outside the first jaw 1101 and the second jaw 1102, one or more clamps 1146 and 1147 of the operation member 1140 may be moved forward so that the stapling process is smoothly performed. In other embodiments, the cartridge 1500 may have a case 1520 corresponding to the bottom, and the case 1520 may be arranged in the first jaw 1101.

In some embodiments, the operation member 1140 may be used together with a wedge WDG. For example, the wedge WDG may be prepared separately from the operation member 1140 and arranged adjacent to the operation member 1140 in the first jaw 1101. In some embodiments, the operation member 1140 and the wedge WDG may be formed integrally with each other. The wedge WDG may be arranged on at least one side of a main body 1142 and may be formed to have a certain inclined surface. For example, the wedge WDG may be formed to be inclined to some extent in the extension direction of the end tool 1100. For example, the height of a proximal end 1101p of the first jaw 1101 may be formed to be higher than the height of the distal end 1101d.

The wedge WDG may be formed to be in sequential contact with a withdrawal member 1535 or a plurality of staples 1530 arranged in the cartridge 1500 and may sequentially push and raise the staples 1530.

The plurality of fixed pulleys 1120 may be arranged in the first jaw 1101 to be closer to the front of the cartridge 1500, that is, closer to the distal end 1101d of the first jaw 1101 than the cartridge 1500. For example, the plurality of fixed pulleys 1120 may be arranged in a front space 1101c of the first jaw 1101, and details thereof are described below.

In some embodiments, the end tool 1100 of the surgical instrument may include one or more members, for example, a joint member, which connects the jaw 1103 to the connection part 1400. In some embodiments, the end tool 1100 may include an end tool hub 1108 and a pitch hub 1107.

The end tool hub 1108 may be arranged to connect the end tool 1100 to the straight portion 1401 of the connection part 1400.

For example, a pulley shaft JX4 may correspond to the end tool hub 1108, and the pulley shaft JX4 may be a pitch rotation shaft. As a specific example, the end tool 1100 may perform a vertical rotation motion around the pulley shaft JX4 with respect to the drawing. In some embodiments, one or more pulleys may be arranged adjacent to the pulley shaft JX4.

The end tool hub 1108 may have a bar shape protruding long from the surface corresponding to the connection part 1400, for example, the center of the disk-shaped main area, and the pulley shaft JX4 and another pulley shaft JX5 may additionally correspond to the bar area.

The pitch hub 1107 may be connected to the end tool hub 1108 and the jaw 1103. The pitch hub 1107 may be axially coupled to the end tool hub 1108 with respect to the pulley shaft JX4. The pitch hub 1107 may be rotated around the pulley shaft JX4 while being connected to the end tool hub 1108. For example, the end tool 1100 may perform a pitch motion by rotating the pitch hub 1107 around the pulley shaft JX4 with respect to the end tool hub 1108.

In some embodiments, the jaw 1103 of the end tool 1100 may be axially coupled with the pitch hub 1107 with respect to a pulley shaft JX1. The jaw 1103 may be rotated around the pulley shaft JX1 while being connected to the pitch hub 1107. For example, the jaw 1103 of the end tool 1100 may perform a yaw motion by rotating around the pulley shaft JX1 with respect to the pulley shaft JX1.

Consequently, the yaw motion of the end tool 1100 may include a motion in which the jaw 1103 is rotated around the pulley shaft JX1 with respect to the pitch hub 1107, and the pitch motion of the end tool 1100 may include a motion in which the pitch hub 1107 is rotated around the pulley shaft JX4 with respect to the end tool hub 1108, and thus, the jaw 1103 coupled to the pitch hub 1107 is rotated together with the pitch hub 1107.

The pitch hub 1107 may include a first hub 1107a and a second hub 1107b.

The first hub 1107a of the pitch hub 1107 may be connected to the jaw 1103. For example, the first hub 1107a may be elongated to be connected to an area of the first jaw 1101. As a specific example, the first hub 1107a may include two parallel bar shapes opposite each other, and the area of the first jaw 1101 may be arranged and coupled between the two parallel bar shapes.

The second hub 1107b of the pitch hub 1107 may be connected to the end tool hub 1108. For example, the second hub 1107b may include two parallel bar shapes opposite each other, and an area of the end tool hub 1108 may be arranged and coupled between the two parallel bar shapes.

As described above, another pulley shaft JX5 which is spaced apart from the pulley shaft JX4 and closer to the connection part 1400 than the pulley shaft JX4 may be arranged in the end tool hub 1108. The pulley shaft JX4 and the pulley shaft JX5 may include shafts arranged parallel to each other.

Another pulley shaft JX2 which is adjacent to and parallel to the pulley shaft JX1 may be arranged in the pitch hub 1107. A pulley shaft JX3 and the pulley shaft JX4 in a direction different from the pulley shaft JX1 and the pulley shaft JX2, for example, in a direction crossing or perpendicular to the pulley shaft JX1 and the pulley shaft JX2, may be sequentially arranged in a direction toward the connection part 1400 (or in a direction away from the operation member).

The pulley shaft JX4 may be a pitch motion shaft of the end tool 1100, and the pulley shaft JX1 may be a yaw motion shaft.

The pulley shaft JX3 and the pulley shaft JX5 may each be a pitch auxiliary pulley shaft, and the pulley shaft JX2 may be a yaw auxiliary pulley shaft. At least one area of one or more driving wires, for example, a wire which transmits the driving force for the pitch motion or a wire which transmits the driving force for the yaw motion, may be in contact with or wound around the pulley shafts JX1, JX2, JX3, JX4, and JX5.

The pulley shafts JX2, JX3, and JX5 adjacent to the pulley shaft JX4, which is the pitch motion shaft, and the pulley shaft JX1, which is the yaw motion shaft, may control a path along which the driving wires are wound around the pulley shaft JX4 and the pulley shaft JX1, so as to ensure the efficiency of the arrangement of the driving wires and the stability of the transmission and path of power through the driving wires.

In some embodiments, at least one area of the forward wire 1110 may be in contact with or wound around the pulley shafts JX1, JX2, JX3, JX4, and JX5.

Further details of the arrangement of the pulley shafts JX1, JX2, JX3, JX4, and JX5 are described below.

One or more switching pulley shafts AX1 and AX2 may be arranged in the end tool 1100, and one or more pulleys corresponding to the switching pulley shafts AX1 and AX2 may be arranged.

For example, the switching pulley shafts AX1 and AX2 may be arranged in a direction close to the jaw 1103, as a specific example, the proximal end 1101p of the first jaw 1101 and may be arranged closer to the distal end 1101d of the first jaw 1101 than the pulley shafts JX1, JX2, JX3, JX4, and JX5 described above.

The pulley shafts AX1 and AX2 may be shafts formed in parallel to each other. Because the pulley shafts AX1 and AX2 are arranged so that the front-and-rear positions between the pulley shafts AX1 and AX2 are different from each other, the switching pulley shaft AX1 and the switching pulley shaft AX2 may be sequentially arranged with respect to the distal end 1101d of the first jaw 1101 and some areas may overlap each other.

At least one area of the forward wire 1110 may be wound around or in contact with the switching pulley shafts AX1 and AX2, and the switching pulley shafts AX1 and AX2 may organize and guide a path before the forward wire 1110 enters the pulley shafts JX1, JX2, JX3, JX4, and JX5. Further details of the arrangement of the switching pulley shafts AX1 and AX2 are described below.

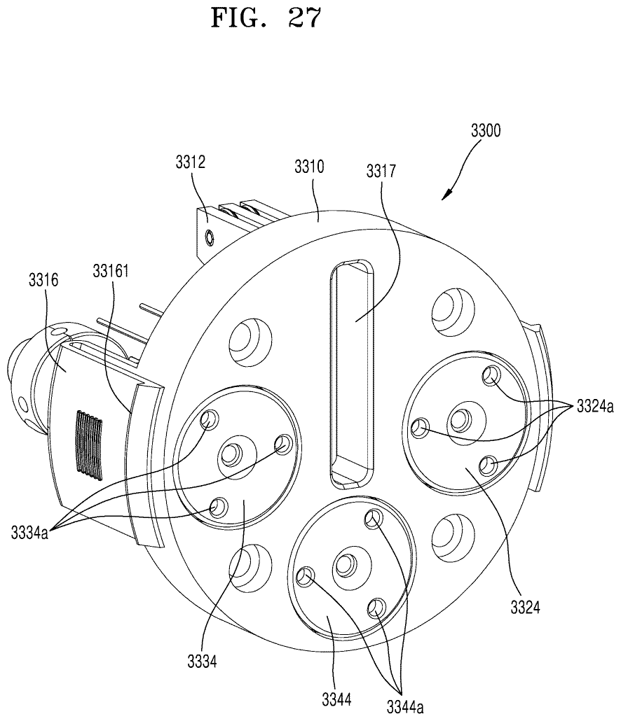

As illustrated in FIG. 27, the first forward wire 1111 and the second forward wire 1112 may be wound to correspond to the first fixed pulley 1121 and the second fixed pulley 1122 in the first jaw 1101 to change direction, and may be connected to the rear of the end tool 1100 through at least one area of the switching pulley shafts AX1 and AX2 and the pulley shafts JX1, JX2, JX3, JX4, and JX5 and may extend up to the manipulation part 1200 through the connection part 1400, so as to be precisely controlled. Accordingly, precise motion control of the operation member 1140 may be easily implemented, and details thereof are described below.

The jaw 1103 of the end tool 1100 is described in more detail.

The second jaw 1102 may be formed entirely in an elongated bar shape. For example, the second jaw 1102 may be formed in a bar shape to correspond to at least one area of the first jaw 1101.

The proximal end 1102p of the second jaw 1102 may include an area coupled to the first jaw 1101. For example, the second jaw 1102 may be formed to be rotatable around the shaft JX of the proximal end 1102p.

The second jaw 1102 may have various forms. As a specific example, a plurality of anvil grooves may be formed in at least one area of the surface of the second jaw 1102 facing the first jaw 1101, and the anvil grooves may include a shape corresponding to the shape of the staples 1530.

The anvil grooves of the second jaw 1102 may serve as a support to bend the staples 1530 when the operation member 1140 pushes and raises the staples 1530 in the stapling motion.

The second jaw 1102 may include a guide groove 1102a. The guide groove 1102a may have a shape which is elongated along the longitudinal direction of the second jaw 1102.

The guide groove 1102a may be formed to guide the operation member 1140 and may be a groove which passes through an area facing the operation member 1140. Accordingly, an area of the operation member 1140, for example, at least one area of the main body 1142 of the operation member 1140, or the first clamp 1146 connected thereto may pass through the guide groove 1102a and be ejected to the outside of the second jaw 1102. When the operation member 1140 is moved forward, the first clamp 1146 may pass through the guide groove 1102a of the second jaw 1102, be exposed to the outside of the second jaw 1102, and be in contact with the upper surface of the second jaw 1102 or apply pressure to the upper surface of the second jaw 1102. By moving the operation member 1140, the first clamp 1146 may apply pressure to the upper surface of the second jaw 1102, and the second clamp 1147 to be described below may apply pressure to the lower surface of the first jaw 1101, so that a gap between the second jaw 1102 and the first jaw 1101 becomes narrow, allowing the second jaw 1102 to naturally maintain a close state with respect to the first jaw 1101.

Referring to FIGS. 3, 4, and the like, the first jaw 1101 may be formed entirely in an elongated bar shape. A rotation shaft may be arranged at the proximal end to enable a rotation motion. The rotation shaft may correspond to the rotation shaft JX formed in the second jaw 1102 described above. In some embodiments, the cartridge 1500 may be accommodated on the side closer to the distal end 1101d than the rotation shaft.

For example, the first jaw 1101 may be formed entirely in the form of a hollow box, of which one surface (upper surface) is removed, such that a cartridge accommodation part 1101a capable of accommodating the cartridge 1500 may be formed inside the first jaw 1101. For example, the first jaw 1101 may be formed in an approximately “U” shape in cross section.

A guide groove 1101h may be formed on the bottom surface of the first jaw 1101, that is, on the bottom surface facing the upper open area in which one surface is removed. For example, the guide groove 1101h may be formed to guide a linear motion of the operation member 1140.

The guide groove 1101h may be formed to guide the operation member 1140 and may be a groove which passes through an area facing the operation member 1140. Accordingly, an area of the operation member 1140, for example, at least one area of the main body 1142 of the operation member 1140, or the second clamp 1147 connected thereto may pass through the guide groove 1101h and be ejected to the outside of the first jaw 1101. When the operation member 1140 is moved forward, the second clamp 1147 may pass through the guide groove 1101h of the first jaw 1101, be exposed to the outside of the first jaw 1101, and be in contact with the lower surface of the first jaw 1101 or apply pressure to the lower surface of the first jaw 1101. By moving the operation member 1140, the second clamp 1147 may apply pressure to the lower surface of the first jaw 1101, and the first clamp 1146 may apply pressure to the upper surface of the second jaw 1102, so that a gap between the second jaw 1102 and the first jaw 1101 becomes narrow, allowing the second jaw 1102 to naturally maintain a close state with respect to the first jaw 1101.

In other embodiments, the first jaw 1101 may include a window 1101b. After the operation member 1140 is operated or after the end tool 1100 is used, the second clamp 1147 of the operation member 1140 may correspond to the window 1101b and release the coupled state between the first jaw 1101 and the operation member 1140.

The first jaw 1101 may include the front space 1101c in front of the cartridge accommodation part 1101a.

For example, the front space 1101c may be arranged closer to the distal end 1101d of the first jaw 1101 than the cartridge accommodation part 1101a. The plurality of fixed pulleys 1120 may be arranged in the front space 1101c. For example, the first fixed pulley 1121 and the second fixed pulley 1122 may be arranged in the front space 1101c.

In describing the disclosure, a portion closer to the user side, that is, a portion close to the manipulation part, is described as a proximal end, and a portion farther from the user side, that is, a portion close to the end tool, is described as a distal end.

For example, a portion of the power transmission part closer to the manipulation part is defined as a proximal end of the power transmission part, and a portion of the power transmission part farther from the power transmission part, that is, a portion of the power transmission part closer to the end tool, is defined as a distal end of the power transmission part. From another viewpoint, the proximal end of the power transmission part may be described as a portion closer to the connection part, and the distal end of the power transmission part may be described as a portion farther from the connection part.

Hereinafter, the power transmission part of the surgical instrument according to the first embodiment is described.

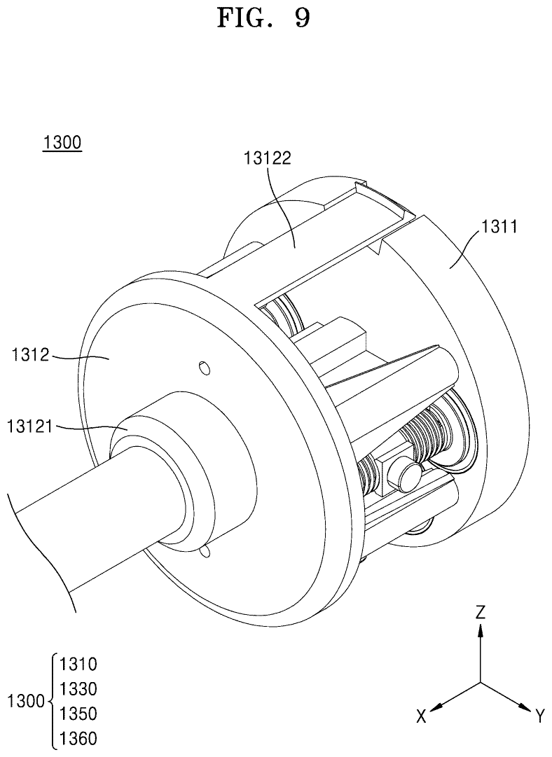

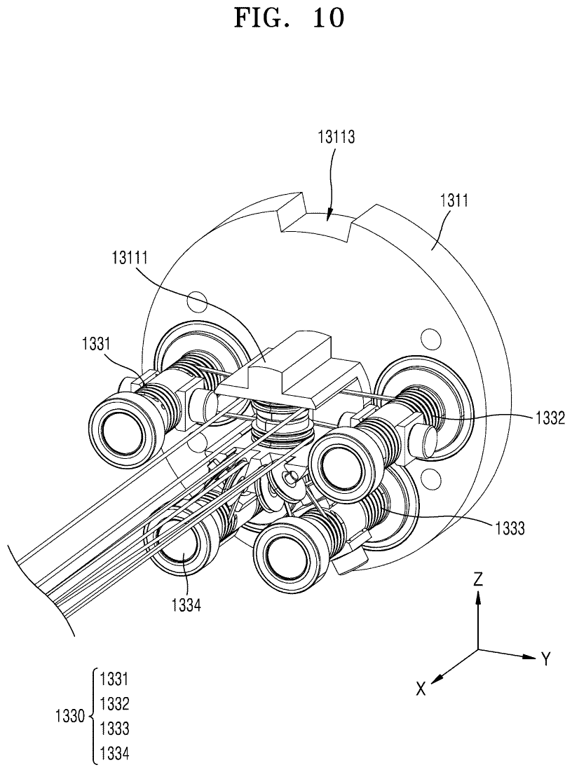

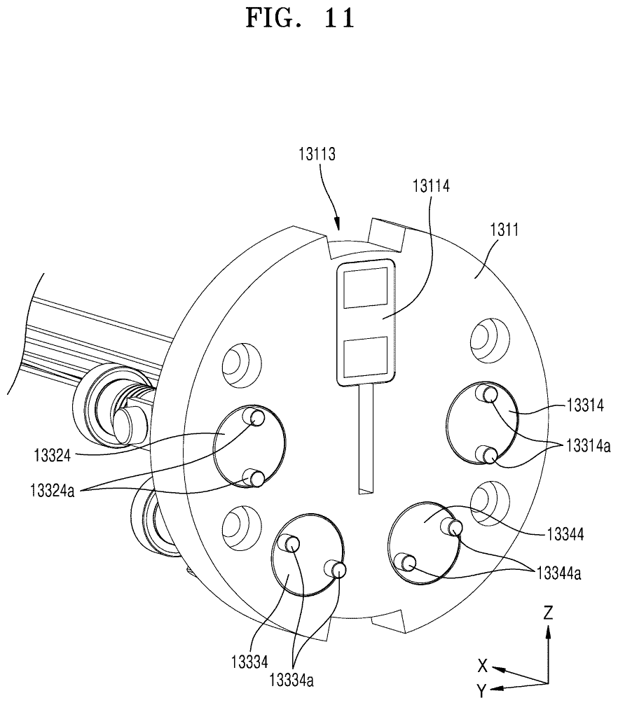

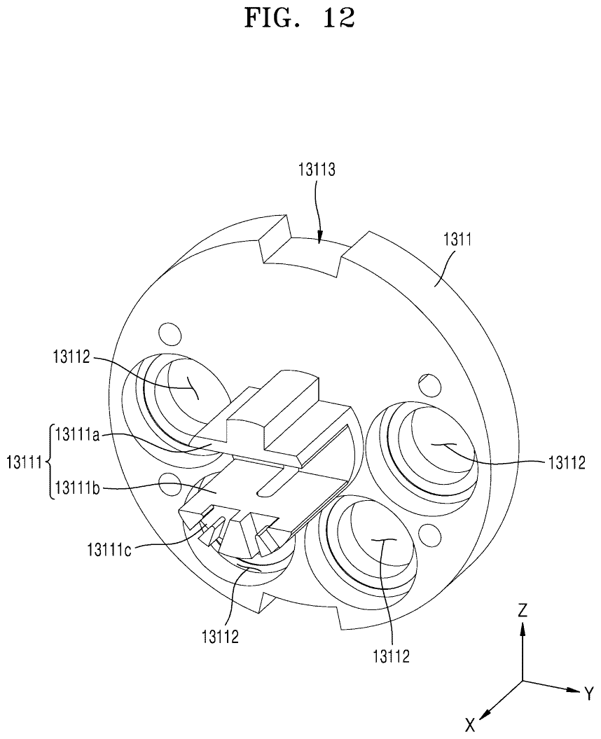

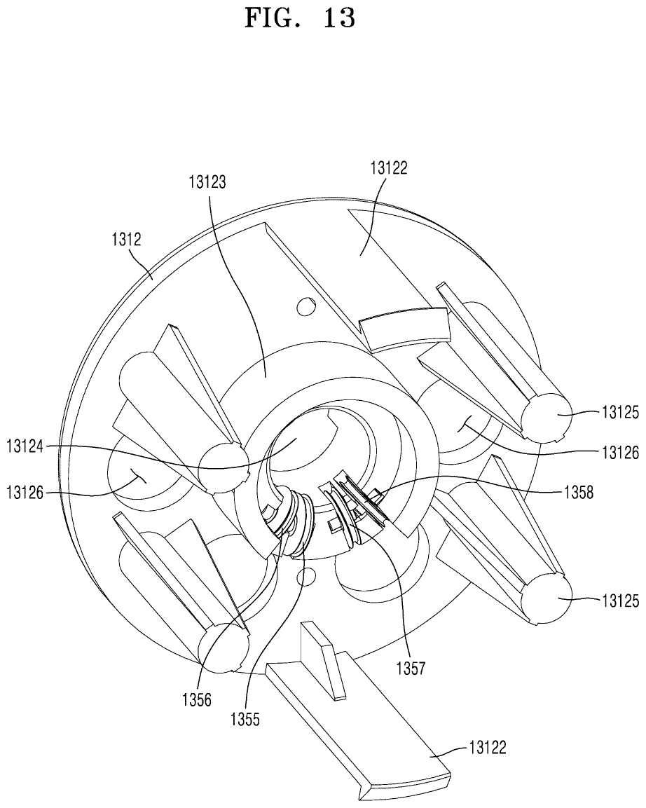

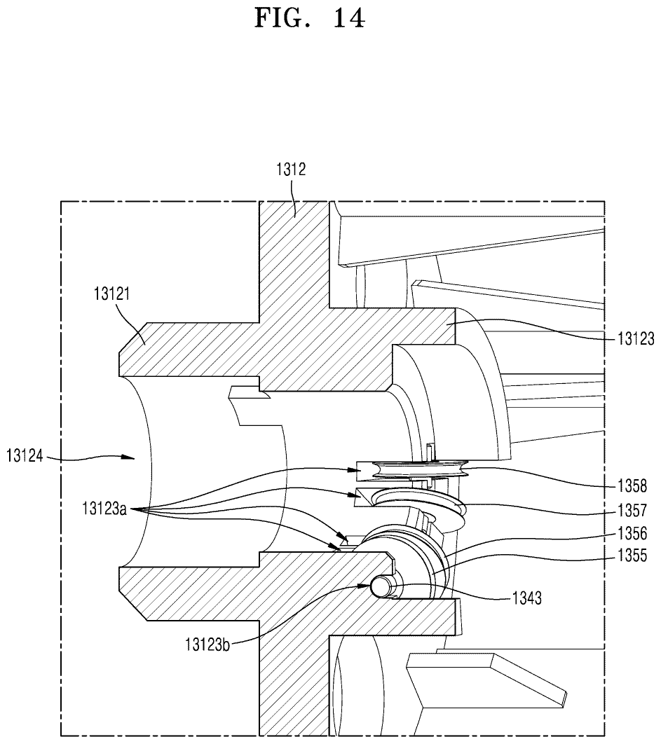

FIG. 9 is a perspective view illustrating the power transmission part 1300 and the shaft (or a connection part) 1400 of FIG. 2. FIG. 10 is a diagram for describing the internal structure of the power transmission part 1300 according to the first embodiment, FIG. 11 is a diagram illustrating the power transmission part 1300 of FIG. 10, when viewed from behind, and FIG. 12 is a perspective view illustrating the first pulley frame 1311 in the power transmission part 1300 of FIG. 10. FIG. 13 is a perspective view illustrating a second pulley frame 1312 in the power transmission part 1300 of FIG. 9, and FIG. 14 is a cross-sectional perspective view illustrating the side cross-section of the second pulley frame 1312 of FIG. 13.

Referring to FIGS. 9 to 14, the power transmission part 1300 of the surgical instrument 1000 according to the first embodiment may include a pulley frame 1310, a driving pulley unit, an auxiliary pulley unit, and a wire unit.

The term “driving pulley unit” is a concept indicating a driving pulley itself or a plurality of driving pulleys. For example, the driving pulley unit may include a plurality of driving pulleys. In describing common contents for each of the plurality of driving pulleys, a representative driving pulley is referred to as a driving pulley 1330 for convenience of explanation.

Similarly, the wire unit may include a plurality of wires. In describing common contents for each of the plurality of wires, a representative wire is referred to as a wire 1360 for convenience of explanation.

In some embodiments, the auxiliary pulley unit may include a plurality of auxiliary pulleys. In describing common contents for each of the plurality of auxiliary pulleys, a representative auxiliary pulley is referred to as an auxiliary pulley 1350 for convenience of explanation.

The pulley frame 1310 may be connected to the power generation part (not shown) described above. For example, the pulley frame 1310 may be coupled to the housing of the manipulation part 1200 and may be connected to the power generation part provided in the manipulation part 1200. For example, the pulley frame 1310 may be connected to a pulley coupling plate of the power generation part to be described below. Accordingly, the pulley frame 1310 may support the driving pulley 1330 so that the driving pulley 1330 described below may be stably connected to a driving motor of the power generation part. However, the concept of the disclosure is not limited thereto, and another device including a drape device may be arranged between the manipulation part 1200 and the power transmission part 1300, so that the power transmission part 1300 may be connected to the other device and thus connected to the power generation part.

In some embodiments, the pulley frame 1310 may accommodate the driving pulley 1330 so that the driving pulley 1330 is axially rotatable. Accordingly, the driving pulley 1330 may be connected to the driving motor and may be rotatable by receiving a driving force generated by the driving motor. Details thereof are described below.

In some embodiments, the pulley frame 1310 may include the first pulley frame 1311 and the second pulley frame 1312. The first pulley frame 1311 and the second pulley frame 1312 may be members connected to each other.

The first pulley frame 1311 may be a portion coupled to the power generation part, as described above. The second pulley frame 1312 may be a portion to which the shaft 1400 is coupled.

From another viewpoint, the pulley frame 1310 may be arranged between the shaft 1400 and the power generation part, the first pulley frame 1311 may be arranged at the proximal end, and the second pulley frame 1312 may be arranged at the distal end.

The first pulley frame 1311 may include a driving pulley coupling part 13112 which accommodates at least a portion of the driving pulley 1330. The driving pulley coupling part 13112 may be formed at a position corresponding to the driving motor (not shown) of the power generation part. The driving pulley coupling part 13112 may be formed in a hollow shape passing through the first pulley frame 1311. For example, the hollow inner surface of the driving pulley coupling part 13112 may be formed to accommodate a bearing 13303 of the driving pulley 1330.

In some embodiments, a plurality of driving pulley coupling parts 13112 may be formed to correspond to the number of driving pulleys 1330. The plurality of driving pulley coupling parts 13112 may be symmetrically arranged with respect to the center of the first pulley frame 1311.

In some embodiments, the first pulley frame 1311 may form an auxiliary pulley fixing part which accommodates at least a portion of the auxiliary pulley 1350.

The auxiliary pulley fixing part may include a center auxiliary pulley fixing part 13111 formed to extend from the first pulley frame 1311 toward the shaft 1400. A center auxiliary pulley to be described below may be accommodated in the center auxiliary pulley fixing part 13111.

For example, the center auxiliary pulley fixing part 13111 may include an upper end portion 13111a and a lower end portion 13111b. The upper end portion 13111a and the lower end portion 13111b of the center auxiliary pulley fixing part 13111 may be formed to extend from the first pulley frame 1311 in a direction parallel to the shaft 1400, and the upper end portion 13111a and the lower end portion 13111b may be formed so that opposite surfaces of the upper end portion 13111a and the lower end portion 13111b are parallel to each other.

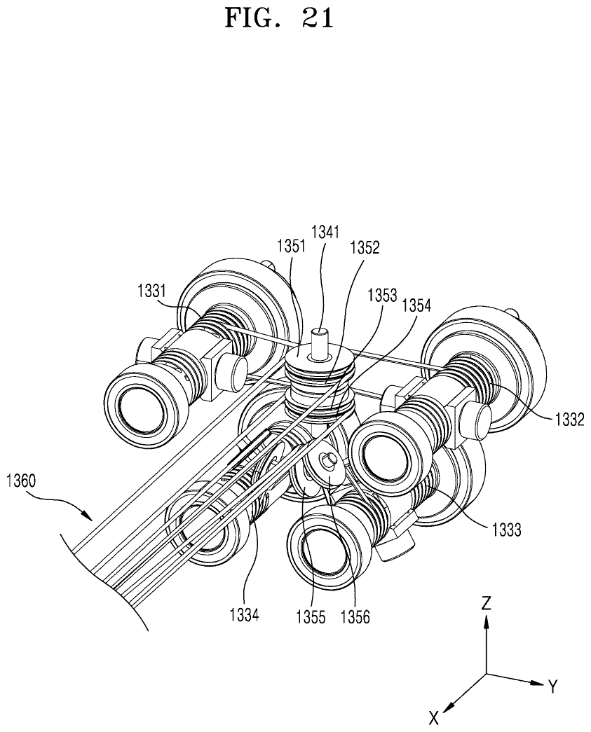

An auxiliary pulley rotation shaft (1341 of FIG. 21) may be coupled between the upper end portion 13111a and the lower end portion 13111b of the center auxiliary pulley fixing part 13111 to accommodate at least a portion of the center auxiliary pulley.

In some embodiments, referring to FIG. 12, a space may be formed at the lower end portion 13111b of the center auxiliary pulley fixing part 13111 to partially accommodate an auxiliary pulley other than the center auxiliary pulley. From another viewpoint, an auxiliary pulley accommodation groove 13111c may be formed at the lower end portion 13111b of the center auxiliary pulley fixing part 13111.

In the embodiment, it has been described that only a portion of the auxiliary pulley is arranged at the lower end portion 13111b of the center auxiliary pulley fixing part 13111, but the concept of the disclosure is not necessarily limited thereto. The auxiliary pulley rotation shaft (1341 of FIG. 21) may be coupled to the lower end portion 13111b of the center auxiliary pulley fixing part 13111 so that auxiliary pulleys 1355, 1356, 1357, and 1358 are rotated around the auxiliary pulley rotation shaft (1341 of FIG. 21).

In some embodiments, the center auxiliary pulley fixing part 13111 may be coupled to an extension portion 13123 of a shaft coupling part of the second pulley frame 1312 to be described below. For example, the center auxiliary pulley fixing part 13111 may be inserted into a hollow formed by the extension portion 13123 of the shaft coupling part. For example, the outermost diameter of the center auxiliary pulley fixing part 13111 may be less than the inner diameter of the extension portion 13123 of the shaft coupling part.

In some embodiments, the first pulley frame 1311 may form one or more fastening member accommodation grooves 13113 on the side circumference. The fastening member accommodation groove 13113 may be formed to accommodate a fastening member 13122 extending from the second pulley frame 1312. For example, the fastening member accommodation groove 13113 may accommodate an end portion of the fastening member 13122. For example, the fastening member accommodation groove 13113 may be formed to have a width greater than a width of the fastening member 13122 and may be formed to have a constant depth so that the end of the fastening member 13122 is movable in a thickness direction.

In some embodiments, the first pulley frame 1311 may further include a data transmission/reception part 13114 electrically connectable to the manipulation part 1200 or another device on a rear surface, which is the opposite surface of the surface opposite the second pulley frame 1312.

The data transmission/reception part 13114 may further include a memory. The memory may store programs and instructions for processing and control by a processor and may also store data input from or output to the device.

For example, the memory may include at least one type of storage medium among flash memory-type memory, multimedia card micro-type memory, card-type memory (for example, secure digital (SD) or extreme digital (XD) memory), random access memory (RAM), static random access memory (SRAM), read-only memory (ROM), electrically erasable programmable read-only memory (EEPROM), and programmable read-only memory (PROM).

The data transmission/reception part 13114 may read and write data, store information related to the end tool 1100, and provide information for preventing reuse. For example, information including the entire length of the reload including the end tool 1100, the shaft 1400, and the power transmission part 1300, the specifications of the reload including the type of the end tool 1100, the number of times the reload has been used, or the like may be stored.

In some embodiments, referring to FIGS. 13 and 14, the second pulley frame 1312 may include a shaft coupling part 13121, an extension portion 13123 of the shaft coupling part 13121, a fastening member 13122, and a driving pulley coupling part 13126.

The shaft coupling part 13121 may protrude from the second pulley frame 1312 to the distal end side. The shaft coupling part 13121 may form a shaft insertion hole 13124 which accommodates at least a portion of the shaft 1400. The shaft insertion hole 13124 may be formed to pass through the second pulley frame 1312. For example, the inner diameter of the shaft coupling part 13121 may be formed to correspond to the outer diameter of the shaft 1400 so that the shaft 1400 may be fitted to the shaft coupling part 13121, and the end portion of the shaft 1400 may be inserted into the shaft coupling part 13121.

The second pulley frame 1312 may have the extension portion 13123 of the shaft coupling part 13121 formed on the opposite surface of the surface where the shaft coupling part 13121 is formed. For example, the extension portion 13123 of the shaft coupling part 13121 may protrude toward the proximal end side. In some embodiments, the extension portion 13123 of the shaft coupling part 13121 may form a hollow inside. From another viewpoint, the shaft coupling part 13121 may be formed on the front and rear surfaces of the second pulley frame 1312, and thus, the shaft coupling part 13121 may be formed as if the shaft coupling part 13121 passes through the second pulley frame 1312. However, the concept of the disclosure is not limited thereto, and the extension portion 13123 of the shaft coupling part 13121 may be formed in various shapes.

In some embodiments, as described above, the extension portion 13123 of the shaft coupling part 13121 may be coupled to the center auxiliary pulley fixing part 13111 of the first pulley frame 1311. In some embodiments, the extension portion 13123 of the shaft coupling part 13121 may be formed to accommodate the center auxiliary pulley fixing part 13111 in the hollow. For example, the inner diameter of the extension portion 13123 of the shaft coupling part 13121 may be formed to be greater than the outermost diameter of the center auxiliary pulley fixing part 13111.

As illustrated in FIG. 14, the extension portion 13123 of the shaft coupling part 13121 may form an auxiliary pulley accommodation groove 13123a and a rotation shaft fixing groove 13123b. The auxiliary pulley accommodation groove 13123a may be formed to accommodate at least a portion of the auxiliary pulleys 1355, 1356, 1357, and 1358, and the rotation shaft fixing groove 13123b may be formed to accommodate auxiliary pulley rotation shafts 1342, 1343, 1344, and 1345.

For example, the auxiliary pulley accommodation groove 13123a may be formed in a slit shape corresponding to the thicknesses of the auxiliary pulleys 1355, 1356, 1357, and 1358).

The rotation shaft fixing groove 13123b may be formed in a structure in which a lateral direction is opened so that the auxiliary pulley rotation shafts 1342, 1343, 1344, and 1345 are fixed in the rotation shaft direction, but are movable in a lateral direction.

The rotating shaft fixing groove 13123b and the auxiliary pulley accommodation groove 13123a may be formed along the circumference of the shaft insertion hole 13124. The auxiliary pulley rotation shafts 1342, 1343, 1344, and 1345 may be arranged perpendicular to the central axis of the shaft 1400, but the auxiliary pulley rotation shafts 1342, 1343, 1344, and 1345 may not be parallel to each other.

In some embodiments, the fastening member 13122 of the second pulley frame 1312 may be a portion extending from the second pulley frame 1312, as described above.

For example, the fastening member 13122 may be formed in a shape with a small thickness relative to the length and width. For example, the fastening member 13122 may be formed in a snap-fit structure.

For example, the end portion of the fastening member 13122 may be formed in a hook shape so as to be coupled to a fastening groove formed in the manipulation part 1200 or another device.

The fastening member 13122 may be formed so as to be at least partially accommodated in the fastening member accommodation groove 13113 of the first pulley frame 1311.

The second pulley frame 1312 may include a frame coupling part 13125 which may be fixedly coupled to the first pulley frame 1311. The frame coupling part 13125 may extend from the second pulley frame 1312 toward the first pulley frame 1311. For example, the frame coupling part 13125 may be formed in a pillar shape, and a plurality of frame coupling parts may be formed along the circumference of the second pulley frame 1312. Accordingly, the frame coupling part 13125 may connect the first pulley frame 1311 to the second pulley frame 1312 so that the first pulley frame 1311 and the second pulley frame 1312 are coupled to each other, and may serve to support the side surface of the pulley frame 1310.

The driving pulley coupling part 13126 formed in the second pulley frame 1312 may be formed at a position corresponding to the driving pulley coupling part 13112 formed in the first pulley frame 1311.

Accordingly, one end portion of the driving pulley 1330 or the rotation shaft of the driving pulley 1330 may be coupled to the first pulley frame 1311, and another end portion of the driving pulley 1330 or the rotation shaft of the driving pulley 1330 may be coupled to the second pulley frame 1312.

For example, bearings 13303 may be coupled to opposite end portions of the driving pulley 1330, and the bearings 13303 may be coupled to the driving pulley coupling part 13126. In other embodiments, the bearings 13303 may be coupled to opposite end portions of the rotation shaft of the driving pulley 1330, and similarly, the bearings 13303 may be coupled to the driving pulley coupling part 13112.

From another viewpoint, it may be described that the driving pulley 1330 is arranged between the first pulley frame 1311 and the second pulley frame 1312, and the first pulley frame 1311 and the second pulley frame 1312 are spaced apart from each other by the length of the driving pulley 1330.

However, the shape and structure of the pulley frame 1310 are not necessarily limited thereto, and the pulley frame 1310 may have various structures for arranging the driving pulley 1330 parallel to the shaft 1400.

Hereinafter, the driving pulley 1330 of the power transmission part 1300 according to the first embodiment is described in detail.

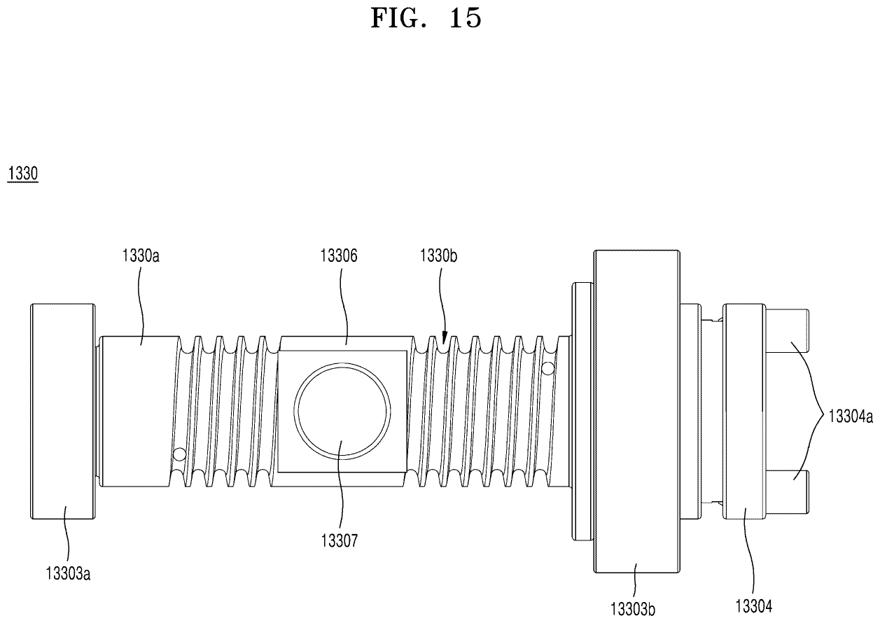

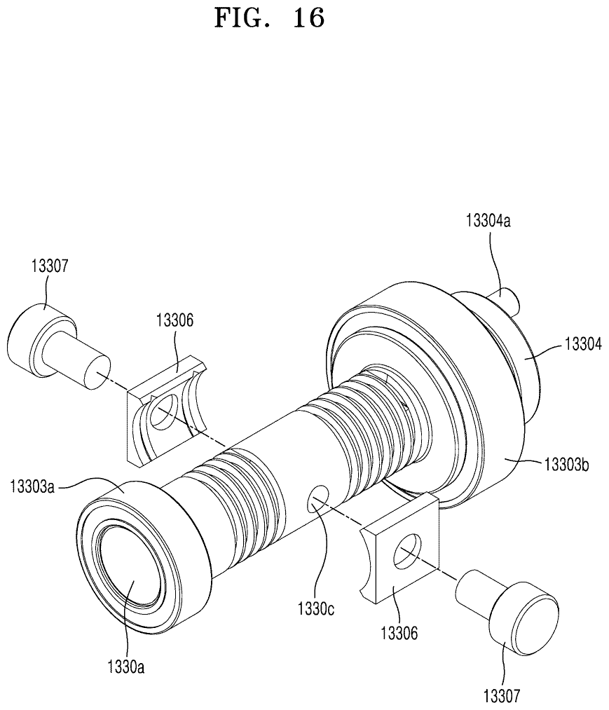

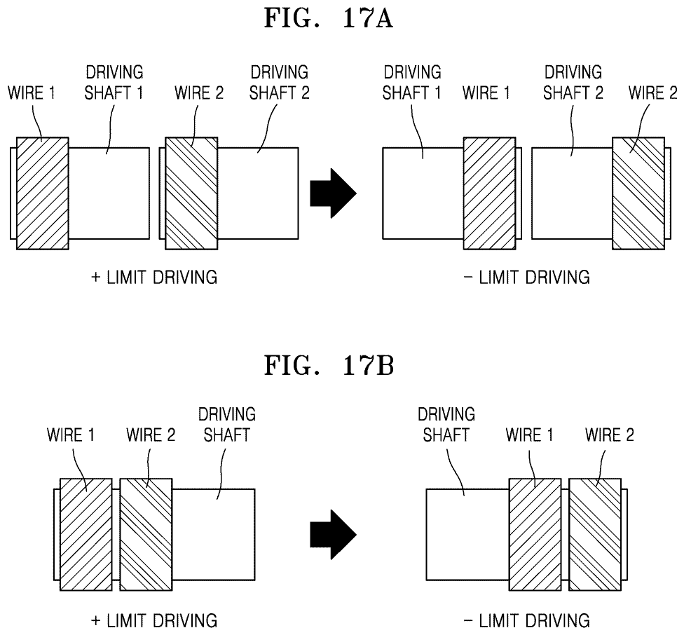

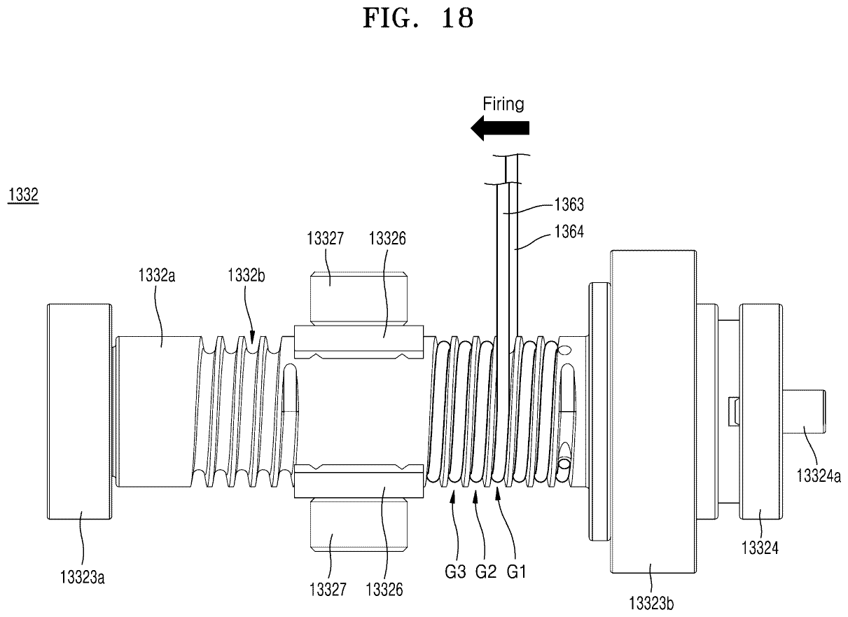

FIG. 15 is a side view illustrating the driving pulley 1330 of the power transmission part 1300 according to the first embodiment, and FIG. 16 is an exploded perspective view of the driving pulley 1330 of FIG. 15. FIGS. 17A and 17B are diagrams for describing a relationship between the driving pulley 1330 and a wire 1360 according to the driving process of the power transmission part 1300 according to the first embodiment. FIGS. 18 to 20 are diagrams for describing a process in which a pair of wires 1360 are wound around or unwound from the driving pulley 1330 of FIG. 15.

Referring again to FIGS. 9 to 11, the driving pulley 1330 may be accommodated in the pulley frame 1310 and may be axially rotated by receiving power generated by the power generation part. The rotation shaft of the driving pulley 1330 may be arranged parallel to the shaft 1400. When a plurality of driving pulleys 1330 are provided, the plurality of driving pulleys 1330 may be arranged parallel to each other.

For example, one end portion of the driving pulley 1330 may be coupled to the first pulley frame 1311, and another end portion of the driving pulley 1330 may be coupled to the second pulley frame 1312. In other embodiments, when the driving pulley 1330 is coupled to a separate rotation shaft, one end portion of the rotation shaft of the driving pulley 1330 may be coupled to the first pulley frame 1311, and another end portion of the rotation shaft of the driving pulley 1330 may be coupled to the second pulley frame 1312.

The driving pulley 1330 of the power transmission part 1300 according to the first embodiment may include firing driving pulleys 1331 and 1332 around which a forward wire and a backward wire are wound, a yaw driving pulley 1333 around which a yaw wire is wound, and a pitch driving pulley 1334 around which a pitch wire is wound. As described below, the firing driving pulleys 1331 and 1332 may include a first firing driving pulley 1331 and a second firing driving pulley 1332.

The firing driving pulleys 1331 and 1332 may be driving pulleys associated with the movement of the operation member 1140 of the end tool 1100, and the forward wire and the backward wire may be wires associated with the movement of the operation member 1140 of the end tool 1100.

The yaw driving pulley 1333 may be a driving pulley associated with the yaw rotation of the end tool 1100, and the yaw wire may be a wire associated with the yaw rotation of the end tool 1100.

The pitch driving pulley 1334 may be a driving pulley associated with the pitch rotation of the end tool 1100, and the pitch wire may be a wire associated with the pitch rotation of the end tool 1100.

The respective driving pulleys are described in detail below.

The wire 1360 may be connected to the side surface of the driving pulley 1330, and the wire 1360 may be wound or unwound according to the rotation of the driving pulley 1330. For example, one end portion of the wire 1360 may be connected to the driving pulley 1330, and another end portion of the wire 1360 may be connected to the end tool 1100, so that power generated by the power generation part may be transmitted to the end tool 1100. Details thereof are described below.

Referring to FIGS. 15 and 16, the driving pulley 1330 of the power transmission part 1300 according to the first embodiment may include a driving pulley body 1330a, a wire fixing part 13306, a pulley plate 13304, and a bearing 13303.

The driving pulley body 1330a may be formed in a cylindrical shape and may form the center of rotation of the driving pulley 1330. The pulley plate 13304 may be coupled to an end portion of the driving pulley body 1330a. Accordingly, the driving pulley 1330 may be axially rotated by receiving a rotation force applied to the pulley plate 13304.

The driving pulley body 1330a may form a groove 1330b in a side surface. For example, the driving pulley 1330 may form a helical groove 1330b shaped like a thread root on an outer surface, and the groove 1330b may be formed so that the wire 1360 is wound around the driving pulley body 1330a of the driving pulley 1330.

The bearing 13303 may be arranged at an end portion or opposite end portions of the driving pulley 1330. The bearing 13303 may support the driving pulley shaft to facilitate the axial rotation of the driving pulley 1330 in the pulley frame 1310.

For example, the bearing 13303a may be inserted into the driving pulley coupling part 13112 formed in the first pulley frame 1311, and the bearing 13303b may be inserted into the driving pulley coupling part 13126 formed in the second pulley frame 1312, so that the driving pulley 1330 may be axially rotated in the pulley frame 1310.

In some embodiments, as a method of fixing the wire 1360 to the driving pulley 1330, a fixing member or a bolt may be used. The fixing member may have various shapes, such as a ball shape or a tube shape, as necessary.

For example, the fixing member (not shown) may be coupled to an end portion of the wire 1360, and the fixing member may fix the end portion of the wire 1360 to the driving pulley 1330 across the driving pulley 1330.

For example, the end portion of the wire 1360 may be fastened to the driving pulley 1330 by using a bolt or the like. For example, the end portion of the wire 1360 may be wound half a turn or one turn under a bolt head, and the bolt may be fastened to the driving pulley 1330 to fix the wire 1360 to the driving pulley 1330.

In some embodiments, the driving pulley 1330 according to an embodiment may have a washer arranged under the bolt so as to more stably press the wire 1360.

In some embodiments, a pair of wires may be wound around the driving pulley 1330. At this time, one wire may be fastened by using the bolt, and the other wire may be fastened by using the fixing member.

For example, an end portion of the one wire may be wound 1.5 turns around the driving pulley 1330, and then, the fixing member may be fastened to the driving pulley 1330, so that the wire is fixed to the driving pulley 1330. By rotating the driving pulley 1330 while the wire is wound to some extent, the wire may be further wound around the driving pulley 1330. Accordingly, the wire may be wound around the driving pulley 1330 while initial tension is applied to the wire.

In this state, the other wire may be wound around the driving pulley 1330 and fixed to the driving pulley 1330 through the bolt. Accordingly, the other wire may be pulled before being wound around the bolt, and thus, the initial tension may be applied. The other wire may be fixed to the driving pulley 1330 through the bolt while the initial tension is applied to the wire.

In some embodiments, the initial tension in the wire may cause the bolt to rotate in a certain direction. At this time, the direction in which the wire is wound is opposite to the direction in which the bolt is fastened. Accordingly, as the wire is pulled, the bolt may be fastened more strongly.

In some embodiments, when the two wires are fixed to the driving pulley 1330 through the fixing members, it may be difficult to apply the initial tension to the wires unless the fixing members are fixed at precise positions. However, as described above, it may be easier to apply the initial tension to the wires by fixing the wire by using the fixing member and fixing the other wire by using the bolt.

Accordingly, the surgical instrument 1000 according to an embodiment may improve productivity by facilitating process control in the process of fixing different wires to the driving pulley 1330.

In some embodiments, the wire fixing part 13306 may be coupled to the side surface of the driving pulley body 1330a. The wire fixing part 13306 may further include a fixing pin 13307 so as to be coupled to the driving pulley body 1330a.

The fixing pin 13307 may serve as the bolt described above, and the wire fixing part 13306 may additionally serve to fix the wire.

For example, the wire fixing part 13306 may have a through hole into which the fixing pin 13307 is inserted. In some embodiments, an insertion hole 1330c into which the fixing pin 13307 is inserted may be formed in the side surface of the driving pulley body 1330a. Accordingly, the fixing pin 13307 may be sequentially coupled to the through hole of the wire fixing part 13306 and the insertion hole 1330c of the driving pulley body 1330a, and thus, the wire fixing part 13306 may be coupled to the driving pulley body 1330a.

The inner surface of the wire fixing part 13306 which is coupled to the driving pulley body 1330a may be formed in a shape corresponding to the shape of the driving pulley body 1330a, so that the inner surface of the wire fixing part 13306 is in close contact with and coupled to the driving pulley body 1330a. For example, the inner surface of the wire fixing part 13306 may be formed in a curved surface corresponding to the side surface of the driving pulley body 1330a. For example, the cross section of the wire fixing part 13306 may form an arc corresponding to a circular shape formed by the cross section of the driving pulley body 1330a.

In some embodiments, one or more grooves may be formed in the inner surface of the wire fixing part 13306 which is coupled to the driving pulley body 1330a. When the wire fixing part 13306 is in close contact with and coupled to the driving pulley body 1330a, a portion of the wire may be arranged in the groove.

Accordingly, an end portion of the wire wound around the driving pulley 1330 may be compressed between the wire fixing part 13306 and the driving pulley body 1330a and fixed to the driving pulley 1330.

The wire fixing part 13306 may be provided as a pair. The pair of wire fixing parts 13306 may be arranged opposite to each other on the side surface of the driving pulley body 1330a.

In some embodiments, the pulley plate 13304 may be coupled to an end portion of the driving pulley body 1330a. The pulley plate 13304 may further include a protrusion 13304a protruding outward. The protrusion 13304a may be fastened to a motor plate provided on a driving motor of another device. Accordingly, the protrusion 13304a may stably fasten the pulley plate 13304 to the motor plate so that the rotation force of the driving motor is transmitted to the driving pulley 1330 through the pulley plate 13304.

However, the concept of the disclosure is not limited thereto. A groove may be formed in the pulley plate 13304, a protrusion may be formed in the motor plate, and the groove and the protrusion may be fastened to each other.

FIGS. 17A and 17B are diagrams for describing a relationship between the driving pulley and the wire according to the driving process of the power transmission part according to the first embodiment. FIGS. 18 to 20 are diagrams for describing a process in which the pair of wires are wound around or unwound from the driving pulley of FIG. 15.

Referring to FIGS. 17A to 20, the wire may be wound around or unwound from the outer circumferential surface of the driving pulley, according to the rotation of the driving pulley.

The wire unit may include a pair of wires which are divided into a first wire and a second wire. The first wire may be wound around the driving pulley 1330 in a first direction, and the second wire may be wound around the driving pulley 1330 in a second direction opposite to the first direction. By rotating the driving pulley 1330 in any one direction, one of the first wire and the second wire may be wound around the driving pulley 1330 and the other of the first wire and the second wire may be unwound from the driving pulley 1330.

For example, when the first wire is wound clockwise around the driving pulley 1330 and the second wire is wound counterclockwise around the driving pulley 1330, when the driving pulley 1330 is rotated clockwise, the first wire may be further wound around the driving pulley 1330 and the second wire may be unwound from the driving pulley 1330. In contrast, when the driving pulley 1330 is rotated counterclockwise, the first wire may be unwound from the driving pulley 1330 and the second wire may be further wound around the driving pulley 1330.

Accordingly, the rotation of the driving pulley 1330 in one direction may cause the first wire and the second wire to be moved in different modes. For example, the rotation of the driving pulley 1330 in one direction may pull one wire and unwind another wire.

For example, the forward wire and the backward wire connected to the firing driving pulleys 1331 and 1332 may be connected to the operation member 1140 of the end tool 1100, and the forward wire and the backward wire connected to the same operation member 1140 may be moved in opposite directions at a ratio of 1:1.

For example, when the firing driving pulleys 1331 and 1332 are rotated in one direction and the forward wire is wound around the firing driving pulleys 1331 and 1332, the operation member 1140 of the end tool 1100 may be moved forward. In contrast, when the firing driving pulleys 1331 and 1332 are rotated in opposite directions and the backward wire is wound around the firing driving pulleys 1331 and 1332, the operation member 1140 of the end tool 1100 may be moved backward.

In some embodiments, when the pair of wires are wound around the driving pulley 1330 in different directions, the positions where the wires are wound may be moved in the lateral direction of the driving pulley 1330 while the wires are wound or unwound.

FIG. 17A illustrates a case where a pair of wires are respectively wound around two driving pulleys, and FIG. 17B illustrates a case where a pair of wires are wound around one driving pulley.

FIGS. 17A and 17B briefly illustrate only the wire at the position unwound from the driving pulley among the wires wound around the driving pulleys.

In general, in the process of winding the wire around the driving pulley, the driving pulley is separated into at least two parts, as illustrated in FIG. 17A. When the wires are respectively fixed to the two parts and then the two parts are rotated in opposite directions, the wire having a spare length is wound around the driving pulley and initial tension is applied to the wire. After that, the two parts are fixed relative to each other. However, in such a case, a gap may occur between the two parts and it may be difficult for the wire to cross from a part to another part beyond the gap when the wire is wound around the driving pulley. For example, when threads formed in the two parts are misaligned and not continuous in a state where the two parts are coupled to each other, it may be difficult for the wire to cross from a part to another when the wire is wound around the driving pulley.

For example, as illustrated in FIG. 17A, wire 1 and wire 2 may be respectively wound around driving shaft 1 and driving shaft 2. At this time, wire 1 may be wound from the left end portion of driving shaft 1, and wire 2 may be wound from the right end portion of driving shaft 2.

First driving (+Limit driving in FIGS. 17A and 17B) may be a state where wire 1 is wound minimally around driving shaft 1 and wire 2 is wound maximally around driving shaft 2. Second driving (−Limit driving in FIGS. 17A and 17B) may be a state where wire 1 is wound maximally around driving shaft 1 and wire 2 is wound minimally around driving shaft 2.

In the process of performing driving from the first driving (+Limit driving) where the wire is located up to the left end portion of the driving shaft to the second driving (−Limit driving) where the wire is located up to the right end portion of the driving shaft on the drawing, the position where each wire is wound around the driving pulley is moved laterally. For example, wire 1 is wound around driving shaft 1, and wire 2 is unwound from driving shaft 2.

At this time, it may be difficult for wire 1 to cross driving shaft 2 beyond a gap formed between driving shaft 1 and driving shaft 2.

In some embodiments, as illustrated in FIG. 17B, two wires may be wound around one driving pulley. For example, wire 1 may be wound from the left end portion of the driving shaft, and wire 2 may be wound from the right end portion of the driving shaft.

First driving (+Limit driving) may be a state where wire 1 is wound minimally around the driving shaft, and wire 2 may be wound maximally around the driving shaft without overlapping wire 1. Second driving (−Limit driving) may be a state where wire 2 is wound minimally around the driving shaft, and wire 1 may be wound maximally around the driving shaft without overlapping wire 2.

At this time, in the process of driving the driving pulley from the first driving (+Limit driving) to the second driving (−Limit driving), the two wires are laterally moved together while wire 1 is wound and wire 2 is unwound. Accordingly, the two wires may be moved without overlapping each other. Because two wires are wound around one driving shaft, a space required may be reduced.

Hereinafter, the second firing driving pulley 1332 is described as an example.

FIG. 18 is a diagram illustrating a state where a pair of wires are wound around the second firing driving pulley 1332. The pair of wires may be a second backward wire 1363 and a second forward wire 1364.

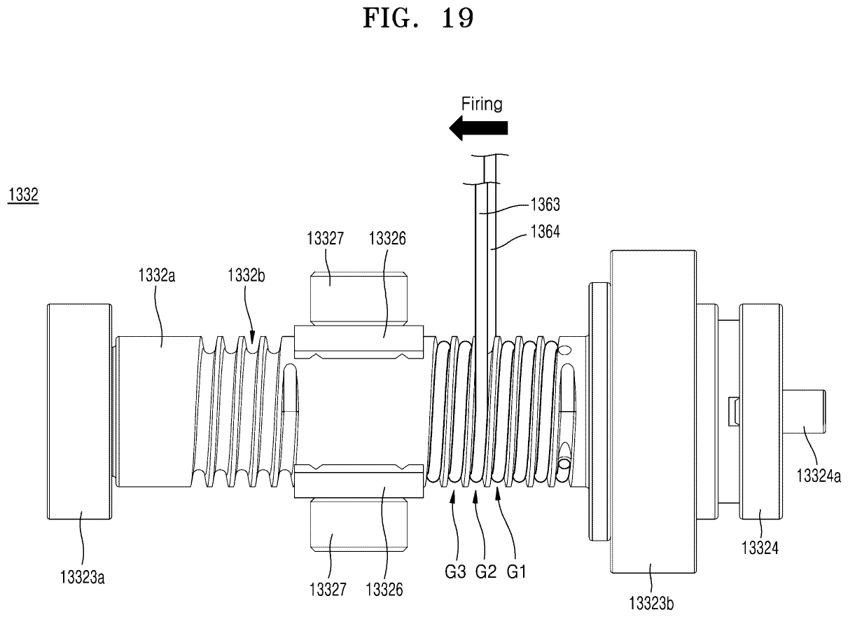

FIG. 19 is a diagram illustrating a state where the second firing driving pulley 1332 of FIG. 18 is rotated in one direction so that the second backward wire 1363 is unwound from the second firing driving pulley 1332 and the second forward wire 1364 is further wound around the second firing driving pulley 1332.

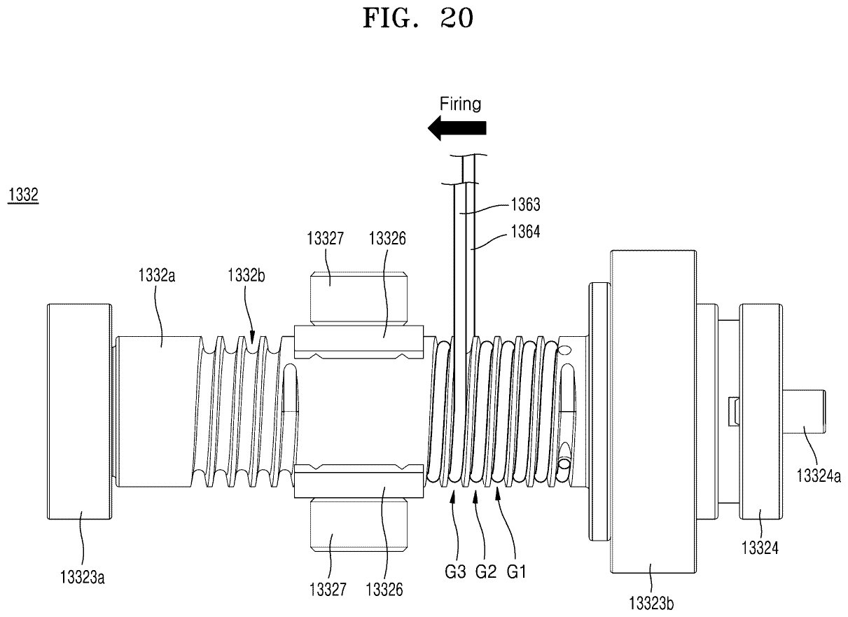

FIG. 20 is a diagram illustrating a state where the second firing driving pulley 1332 of FIG. 19 is rotated in one direction so that the second backward wire 1363 is unwound from the second firing driving pulley 1332 and the second forward wire 1364 is further wound around the second firing driving pulley 1332.

In the process from FIG. 18 to FIG. 20, the second firing driving pulley 1332 may be rotated one turn. For example, whenever the second firing driving pulley 1332 is rotated one turn, the second backward wire 1363 may be unwound one turn from the second firing driving pulley 1332, and the second forward wire 1364 may be wound one turn around the second firing driving pulley 1332.

In other words, the driving pulley 1330 may form a continuous groove 1330b around which the first wire and the second wire are wound, and as the driving pulley 1330 is rotated, a wire may be wound in a place where another wire is unwound.

For example, as illustrated in FIG. 18, the second backward wire 1363 and the second forward wire 1364 are wound around a first groove G1. However, as the driving pulley is rotated in one direction, the second backward wire 1363 and the second forward wire 1364 are wound around a second groove G2. As the driving pulley is further rotated, the second backward wire 1363 and the second forward wire 1364 are wound around a third groove G3.

In other words, in the driving pulley 1330 according to the first embodiment, not only one wire may be wound around the groove 1330b, but also the first wire or the second wire wound around the groove 1330b. From another viewpoint, a pair of wires connected to one driving pulley 1330 may share the groove 1330b with each other.

Hereinafter, the arrangement structure of the driving pulley 1330, the auxiliary pulley 1350, and the wire 1360 in the power transmission part 1300 according to the first embodiment is described in detail.

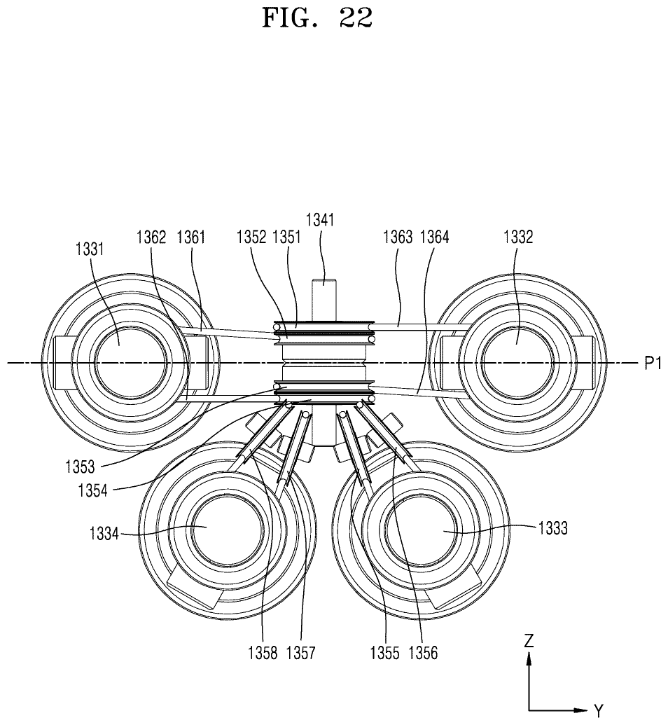

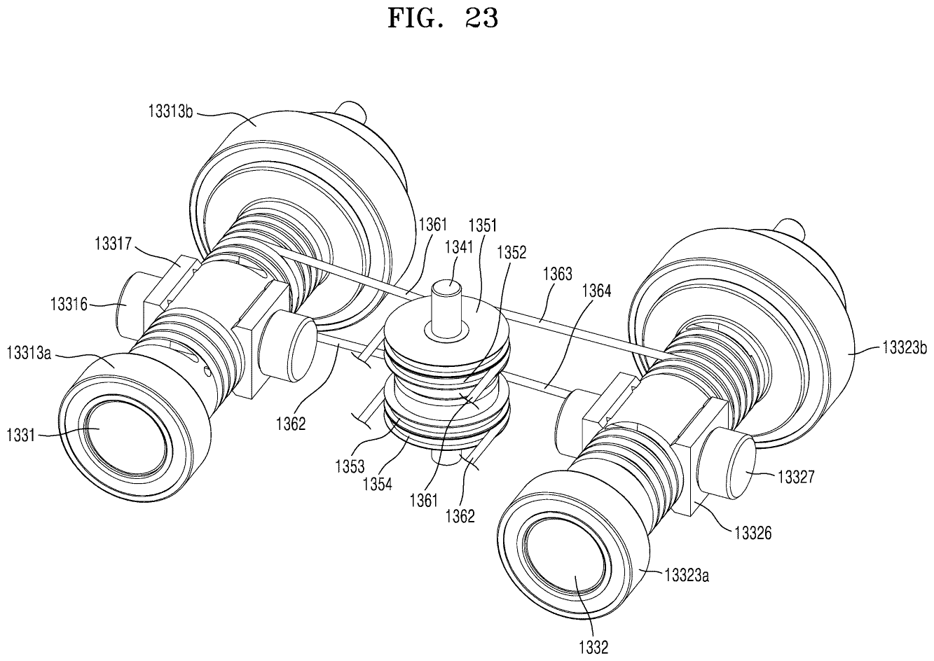

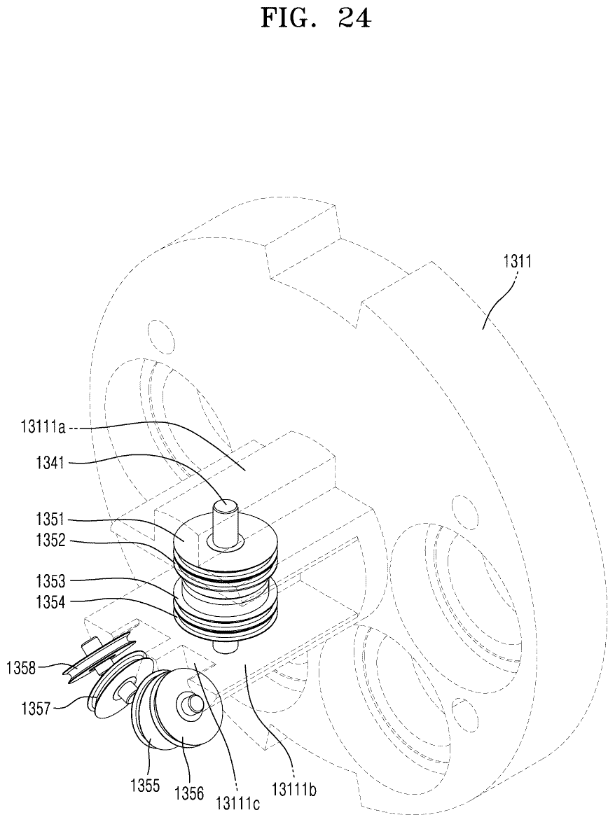

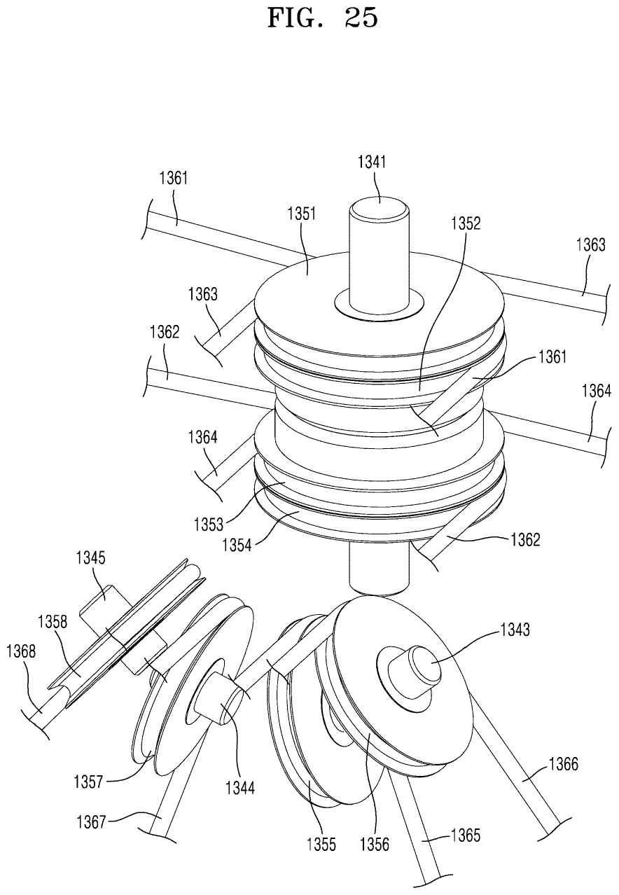

FIG. 21 is a perspective view illustrating a state where the first pulley frame of the power transmission part of FIG. 10 is removed. FIG. 22 is a diagram illustrating the power transmission part of FIG. 21, when viewed from the front in an X-axis direction, and FIG. 23 is a perspective view illustrating wires connected to the driving pulley and the center auxiliary pulley in the power transmission part of FIG. 21. FIG. 24 is a diagram for describing the auxiliary pulley arranged on the first pulley frame in the power transmission part of FIG. 10, and FIG. 25 is a perspective view illustrating a state where the wire is wound around the auxiliary pulley of FIG. 24.

Referring to FIGS. 21 to 23, a driving pulley unit may include firing driving pulleys 1331 and 1332, a yaw driving pulley 1333, and a pitch driving pulley 1334.

The firing driving pulleys 1331 and 1332 may be driving pulleys around which the forward wire and the backward wire are wound. The yaw driving pulley 1333 may be a driving pulley around which the yaw wire is wound. The pitch driving pulley 1334 may be a driving pulley around which the pitch wire is wound.

The driving pulley unit may include a pair of driving pulleys arranged symmetrically with respect to the central axis of the shaft 1400.

The pair of driving pulleys arranged symmetrically with respect to the central axis of the shaft 1400 may be the first firing driving pulley 1331 and the second firing driving pulley 1332.

The yaw driving pulley 1333 and the pitch driving pulley 1334 may be spaced apart from the pair of firing driving pulleys 1331 and 1332 and arranged parallel to the shaft 1400. Although the yaw driving pulley 1333 and the pitch driving pulley 1334 are illustrated as being arranged under the firing driving pulleys 1331 and 1332, the concept of the disclosure is not limited thereto, and the yaw driving pulley 1333 and the pitch driving pulley 1334 may be arranged parallel to the firing driving pulleys 1331 and 1332 in the pulley frame 1310.

Referring to FIGS. 21 to 25, the auxiliary pulley 1350 may partially contact the wire 1360, and may guide the wire 1360 to the shaft 1400 by changing a traveling path of the wire 1360 extending from the driving pulley 1330.

From another viewpoint, the wire 1360 wound around the driving pulley 1330 may pass through the auxiliary pulley 1350 and be arranged in the shaft 1400. The respective wires 1360 may pass through the inside of the shaft 1400 and be connected to the pulley of the end tool 1100.

The auxiliary pulley unit includes at least one pair of auxiliary pulleys. The pair of auxiliary pulleys may be spaced apart from each other to correspond to the diameter of the driving pulley 1330 around which the pair of wires are wound.

The pair of wires may be wound around one driving pulley 1330 and arranged to extend from the driving pulley 1330. The respective wires may be arranged parallel or substantially parallel to each other. At this time, the respective wires extending from the driving pulley 1330 may be sufficiently spaced apart from each other by passing through the respective auxiliary pulleys 1350 spaced apart from each other.

The auxiliary pulley unit may include a center auxiliary pulley arranged adjacent to the central axis of the shaft 1400.

In other words, the center auxiliary pulley may be arranged adjacent to the extension line of the shaft 1400. From another viewpoint, the center auxiliary pulley may be arranged between the pair of firing driving pulleys 1331 and 1332.

The center auxiliary pulley may be arranged in the center auxiliary pulley fixing part 13111 of the first pulley frame 1311.

The center auxiliary pulley may be axially coupled to the center auxiliary pulley rotation shaft 1341, and the center auxiliary pulley rotation shaft 1341 may be coupled to the center auxiliary pulley fixing part 13111.

The center auxiliary pulley rotation shaft 1341 to which the center auxiliary pulley is axially coupled may be perpendicular to the central axis of the shaft 1400. In some embodiments, the center auxiliary pulley rotation shaft 1341 may be perpendicular to an imaginary plane P1 which includes the rotation shafts of the pair of driving pulleys arranged symmetrically with respect to the central axis of the shaft 1400.

Because the center auxiliary pulley is rotated with respect to the center auxiliary pulley rotation shaft 1341, the rotation plane on which the center auxiliary pulley is rotated may be parallel to the imaginary plane P1.

Accordingly, the wire extending from the firing driving pulleys 1331 and 1332 may change a direction by nearly 90° by passing through the center auxiliary pulley and enter the shaft 1400.

Because the auxiliary pulley of the power transmission part 1300 according to the first embodiment is arranged as described above, the wire extending from the inside of the shaft 1400 to the power transmission part 1300 may be stably wound around the driving pulley 1330 arranged parallel to the shaft 1400.

In some embodiments, the center auxiliary pulley may include a first auxiliary pulley 1351, a second auxiliary pulley 1352, a third auxiliary pulley 1353, and a fourth auxiliary pulley 1354, which are axially coupled with respect to the same rotation shaft.

The first auxiliary pulley 1351 and the second auxiliary pulley 1352 may be arranged on a side with respect to the central axis of the shaft 1400, and the third auxiliary pulley 1353 and the fourth auxiliary pulley 1354 may be arranged on another side with respect to the central axis of the shaft 1400.

From another viewpoint, the first auxiliary pulley 1351 and the second auxiliary pulley 1352 may be arranged on a side with respect to the imaginary plane P1 including the rotation shafts of the pair of driving pulleys, and the third auxiliary pulley 1353 and the fourth auxiliary pulley 1354 may be arranged on another side with respect to the imaginary plane P1 including the rotation shafts of the pair of driving pulleys.

From another viewpoint, the first auxiliary pulley 1351 and the second auxiliary pulley 1352 may be arranged close to each other, and the third auxiliary pulley 1353 and the fourth auxiliary pulley 1354 may be arranged close to each other. The first auxiliary pulley 1351 and the second auxiliary pulley 1352 may be spaced apart from the third auxiliary pulley 1353 and the fourth auxiliary pulley 1354.

As described above, in the power transmission part 1300 according to an embodiment, the pair of wires may be wound around one driving pulley 1330. For example, the forward wire and the backward wire may be wound around one firing driving pulleys 1331 and 1332 in different directions.

One of the forward wire and the backward wire may pass through the auxiliary pulley arranged at the upper end, and the other of the forward wire and the backward wire may pass through the auxiliary pulley arranged at the lower end.

For example, the driving pulley unit may include a first driving pulley and a second driving pulley arranged symmetrically with respect to the central axis of the shaft 1400. The first forward wire 1361 and the first backward wire 1362 may be wound around the first driving pulley, and the second forward wire 1364 and the second backward wire 1363 may be wound around the second driving pulley.

For example, the first driving pulley may be the first firing driving pulley 1331, and the second driving pulley may be the second firing driving pulley 1332.

The first forward wire 1361 may be wound around the second auxiliary pulley 1352, the first backward wire 1362 may be wound around the fourth auxiliary pulley 1354, the second forward wire 1364 may be wound around the third auxiliary pulley 1353, and the second backward wire 1363 may be wound around the first auxiliary pulley 1351.

In an embodiment, the first forward wire 1361 may be wound around the first auxiliary pulley 1351, the first backward wire 1362 may be wound around the third auxiliary pulley 1353, the second forward wire 1364 may be wound around the fourth auxiliary pulley 1354, and the second backward wire 1363 may be wound around the second auxiliary pulley 1352.

When the pair of wires wound around one driving pulley are all arranged only at the upper end or the lower end with respect to the central axis of the shaft 1400, one of the wires may be sharply bent when the wire passes through the auxiliary pulley and is wound around the driving pulley.

For example, when looking at the progressing path in which the wire enters the auxiliary pulley from the shaft 1400 and exits from the auxiliary pulley to the driving pulley, even when the wire enters parallel to the rotation plane of the auxiliary pulley, the wire may be bent at a large angle with respect to the rotation plane of the auxiliary pulley when exiting from the auxiliary pulley. Accordingly, the wire exerts a twisting force on the auxiliary pulley, which may cause great friction between the wire and the auxiliary pulley.

However, because the auxiliary pulley 1350 is arranged as described above, the power transmission part 1300 according to an embodiment may reduce a force which the auxiliary pulley 1350 receives from the wire 1360 and may reduce a friction which occurs between the wire 1360 and the auxiliary pulley 1350.

In some embodiments, because the first auxiliary pulley 1351, the second auxiliary pulley 1352, the third auxiliary pulley 1353, and the fourth auxiliary pulley 1354 are axially coupled to one rotation shaft, the power transmission part 1300 according to an embodiment may partially offset a force applied to the center auxiliary pulley rotation shaft 1341.

For example, the first forward wire 1361 and the second forward wire 1364 are moved in opposite directions on the Y-axis, and the first backward wire 1362 and the second backward wire 1363 are moved in opposite directions on the Y-axis. Accordingly, the Y-axis component forces among the forces applied to the rotation shaft may be offset from each other.

In some embodiments, referring again to FIGS. 21 to 25, a pair of yaw wires 1365 and 1366 wound around the yaw driving pulley 1333 and extending from the yaw driving pulley 1333 may pass through yaw auxiliary pulleys 1355 and 1356 and extend toward the shaft 1400. A pair of pitch wires 1367 and 1368 wound around the pitch driving pulley 1334 and extending from the pitch driving pulley 1334 may pass through pitch auxiliary pulleys 1357 and 1358 and extend toward the shaft 1400.

The auxiliary pulleys 1355 and 1356 and the pitch auxiliary pulleys 1357 and 1358 may be coupled to different shafts.

For example, the yaw auxiliary pulleys 1355 and 1356 may be provided as a pair. The pair of yaw auxiliary pulleys 1355 and 1356 may be spaced apart from each other and may be arranged not to be parallel to each other.

For example, the pair of yaw auxiliary pulleys 1355 and 1356 may be arranged closer to each other as the pair of yaw auxiliary pulleys 1355 and 1356 are farther away from the yaw driving pulley 1333.

Similarly, the pitch auxiliary pulleys 1357 and 1358 may be provided as a pair. The pair of pitch auxiliary pulleys 1357 and 1358 may be spaced apart from each other and may be arranged not to be parallel to each other.

For example, the pair of pitch auxiliary pulleys 1357 and 1358 may be arranged closer to each other as the pair of pitch auxiliary pulleys 1357 and 1358 are farther away from the pitch driving pulley 1334.

The yaw auxiliary pulleys 1355 and 1356 and the pitch auxiliary pulleys 1357 and 1358 may be partially accommodated in the auxiliary pulley accommodation grooves 13111c formed at the lower end portion 13111b of the center auxiliary pulley fixing part 13111.

In some embodiments, as described above, the yaw auxiliary pulleys 1355 and 1356 and the pitch auxiliary pulleys 1357 and 1358 may be partially accommodated in the auxiliary pulley accommodation grooves 13123a formed in the second pulley frame 1312. The auxiliary pulley rotation shafts 1342, 1343, 1344, and 1345 may be fixed to the rotation shaft fixing groove 13123b.

Hereinafter, a power transmission part according to a second embodiment is described.

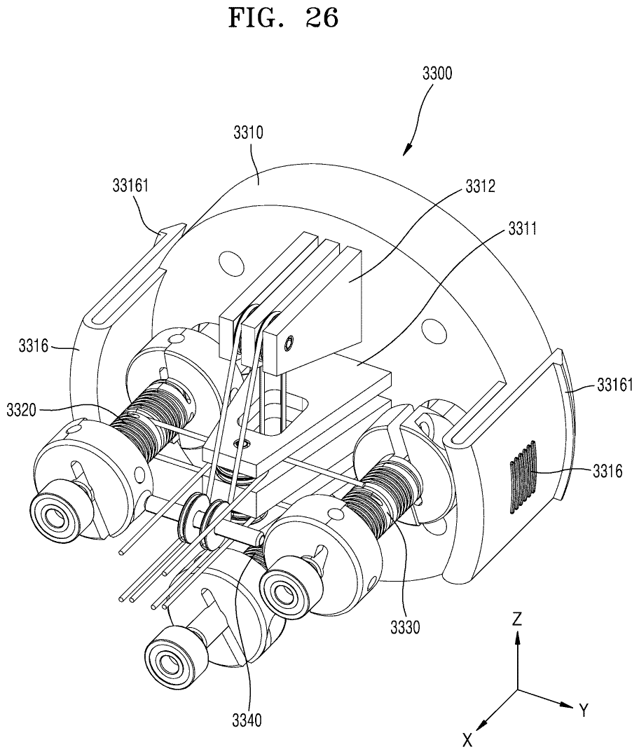

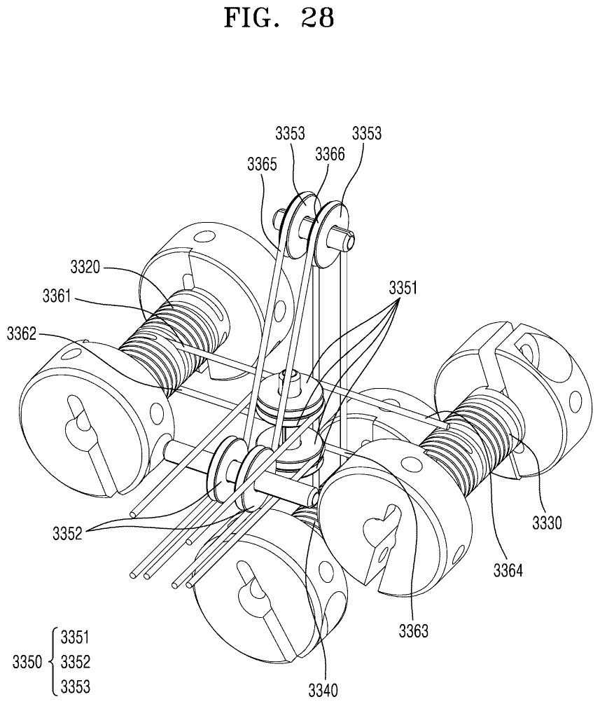

FIG. 26 is a diagram for describing the internal structure of the power transmission part according to the second embodiment. FIG. 27 is a diagram illustrating the power transmission part of FIG. 26, when viewed from behind. FIG. 28 is a diagram for describing an arrangement of a pulley and a wire in the power transmission part of FIG. 26.

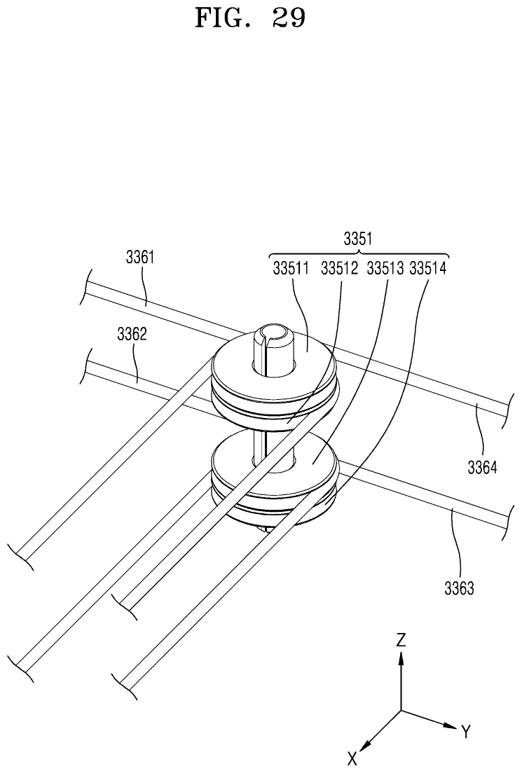

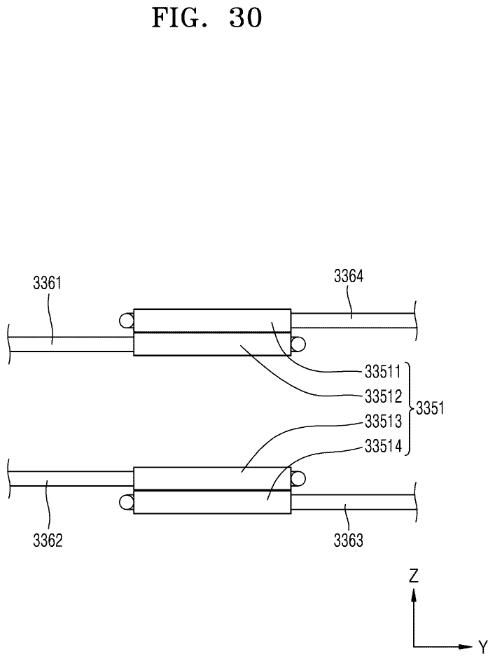

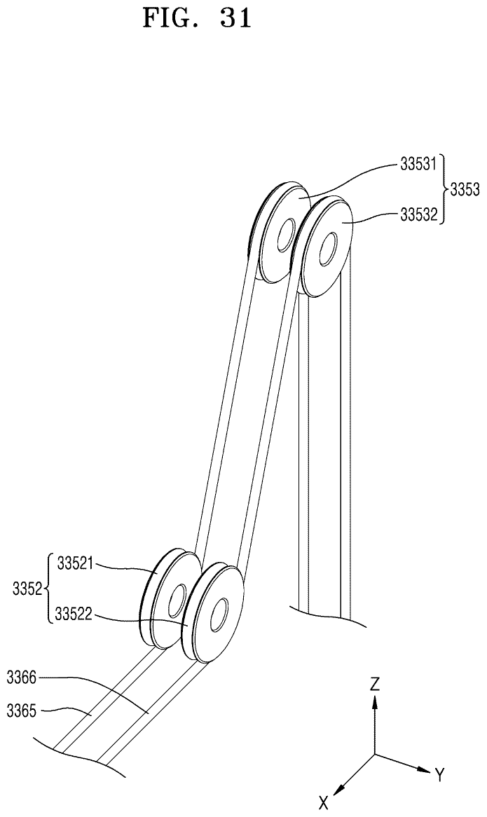

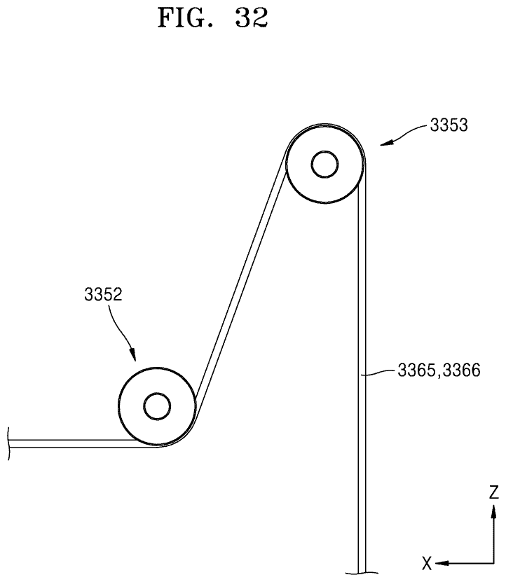

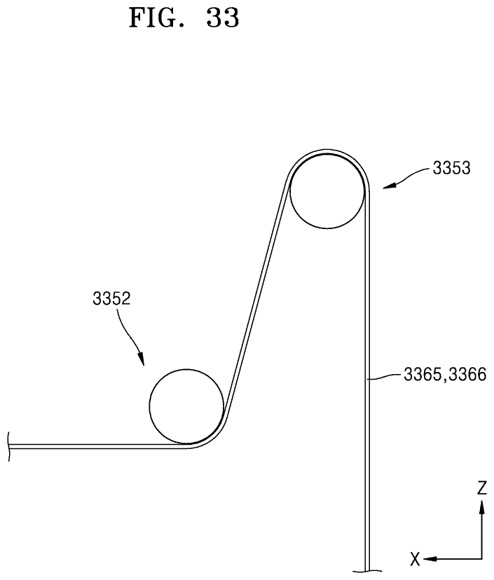

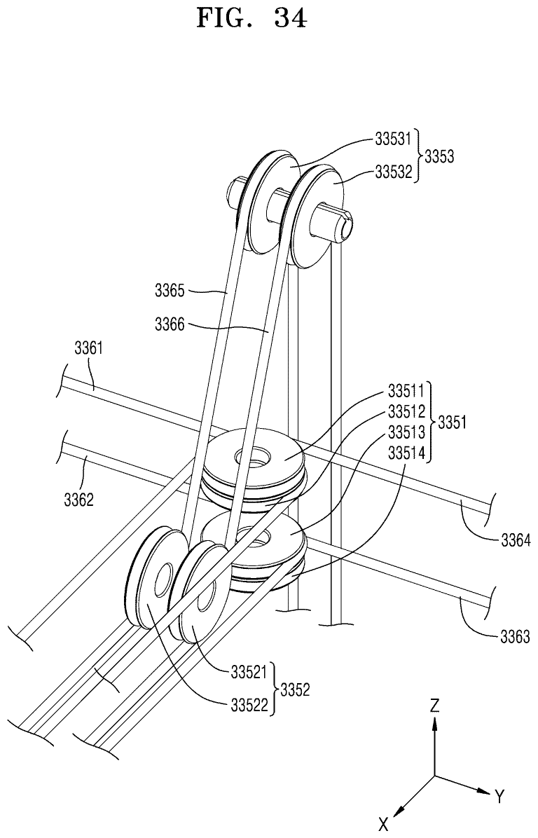

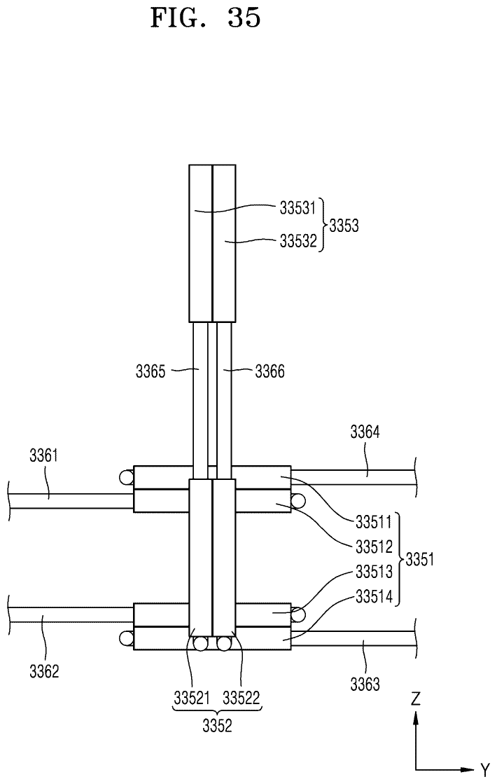

FIG. 29 is a perspective view illustrating a center auxiliary pulley and a wire in the power transmission part of FIG. 28. FIG. 30 is a diagram illustrating the center auxiliary pulley and the wire of FIG. 29, when viewed from the front. FIG. 31 is a perspective view illustrating the auxiliary pulley and the wire in the power transmission part of FIG. 28. FIGS. 32 and 33 are side views illustrating the auxiliary pulley and the wire of FIG. 31, when viewed from a side. FIG. 34 is a perspective view illustrating the arrangement of the auxiliary pulley and the wire in the power transmission part of FIG. 28, excluding the driving pulley. FIG. 35 is a diagram illustrating the auxiliary pulley and the wire of FIG. 34, when viewed from the front.

Referring to FIGS. 26 to 28, the power transmission part 3300 according to the second embodiment may include a pulley frame 3310, at least one pulley, and at least one wire.

The pulley frame 3310 may form the overall frame of the power transmission part 3300.

At least one pulley may be arranged on the pulley frame 3310. The expression that “the pulley is arranged” should be interpreted in a broad sense. For example, the expression that “the pulley is arranged” may mean that the pulley is directly connected to the pulley frame 3310, or may mean that a rotation shaft is installed in the pulley frame 3310 and the pulley is connected to the rotation shaft. For example, the expression that “the pulley is arranged” may mean that a separate member is provided in the pulley frame 3310, a rotation shaft is connected to the separate member, and a pulley is connected to the rotation shaft. For example, the expression that “the pulley is arranged” may mean that a hole is formed in the pulley frame 3310, a rotation shaft is arranged to pass through the hole, and a pulley is installed on the rotation shaft.

The power transmission part 3300 may include at least one pulley.

The pulley is a member around which the wire is wound, and a groove around which the wire is wound may be formed in the pulley.

In an embodiment, the power transmission part 3300 may include a yaw driving pulley 3320, a pitch driving pulley 3330, and a firing driving pulley 3340. In some embodiments, the power transmission part 3300 may include at least one yaw wire 3361 and 3362, at least one pitch wire 3363 and 3364, and firing wires 3365 and 3366.

The yaw driving pulley 3320 may be a pulley associated with a yaw rotation of an end tool 1100, and the yaw wires 3361 and 3362 may be wires associated with the yaw rotation of the end tool 1100.

The yaw driving pulley 3320 may be arranged in an area of the pulley frame 3310.

The yaw wires 3361 and 3362 may be arranged on the end tool 1100 and may extend from the pulley associated with the yaw rotation of the end tool 1100 toward the yaw driving pulley 3320.

In an embodiment, the yaw wires 3361 and 3362 may be provided as a pair. As described below, the yaw wires 3361 and 3362 may be respectively connected to the upper side and the lower side of the yaw driving pulley 3320.

In other embodiments, the yaw wires 3361 and 3362 may include a first yaw wire 3361 and a second yaw wire 3362. The first yaw wire 3361 may extend upward from the yaw driving pulley 3320 with respect to FIG. 28 and may be connected to the yaw driving pulley 3320. The second yaw wire 3362 may extend downward from the yaw driving pulley 3320 with respect to FIG. 28 and may be connected to the yaw driving pulley 3320.

After the yaw wires 3361 and 3362 are connected to the yaw driving pulley 3320, the yaw wires 3361 and 3362 may be wound around or unwound from the yaw driving pulley 3320 when the yaw driving pulley 3320 is rotated. For example, when the yaw driving pulley 3320 is rotated in one direction, one yaw wire may be wound around the yaw driving pulley 3320, and another yaw wire may be unwound from the yaw driving pulley 3320. For example, when the yaw driving pulley 3320 is rotated clockwise with respect to FIG. 28, the first yaw wire 3361 may be unwound from the yaw driving pulley 3320, and the second yaw wire 3362 may be wound around the yaw driving pulley 3320. In contrast, when the yaw driving pulley 3320 is rotated counterclockwise with respect to FIG. 28, the first yaw wire 3361 may be wound around the yaw driving pulley 3320, and the second yaw wire 3362 may be unwound from the yaw driving pulley 3320.

From another viewpoint, it may be stated that, when the yaw driving pulley 3320 is rotated, the pair of yaw wires 3361 and 3362 are moved in opposite directions with respect to the yaw driving pulley 3320.

As such, when the yaw driving pulley 3320 is rotated to move the pair of yaw wires 3361 and 3362 in different directions, the pulley connected to the yaw wires 3361 and 3362 on the end tool 1100 side is rotated in the corresponding direction. Accordingly, the pulley connected to the yaw wires 3361 and 3362 on the end tool 1100 side may implement the yaw rotation of the end tool 1100 while rotating in one direction.

The pitch driving pulley 3330 may be a pulley associated with a pitch rotation of the end tool 1100, and the pitch wires 3363 and 3364 may be wires associated with the pitch rotation of the end tool 1100.

The pitch driving pulley 3330 may be arranged in an area of the pulley frame 3310.

In an embodiment, the pitch driving pulley 3330 may be arranged on an opposite side of the yaw driving pulley 3320. In other embodiments, the pitch driving pulley 3330 and the yaw driving pulley 3320 may be arranged symmetrically on opposite sides with respect to the center of the pulley frame 3310. Accordingly, as described below, the yaw wires 3361 and 3362 and the pitch wires 3363 and 3364 extending to pass through the auxiliary pulley 3350 may substantially vertically approach the yaw driving pulley 3320 and the pitch driving pulley 3330.

The pitch wires 3363 and 3364 may be arranged on the end tool 1100 and may extend from the pulley associated with the pitch rotation of the end tool 1100 toward the pitch driving pulley 3330.

In an embodiment, the pitch wires 3363 and 3364 may be provided as a pair. As described below, the pitch wires 3363 and 3364 may be respectively connected to the upper side and the lower side of the pitch driving pulley 3330.

In other embodiments, the pitch wires 3363 and 3364 may include a first pitch wire 3363 and a second pitch wire 3364. The first pitch wire 3363 may extend downward from the pitch driving pulley 3330 with respect to FIG. 28 and may be connected to the pitch driving pulley 3330. The second pitch wire 3364 may extend upward from the pitch driving pulley 3330 with respect to FIG. 28 and may be connected to the pitch driving pulley 3330.

After the pitch wires 3363 and 3364 are connected to the pitch driving pulley 3330, the pitch wires 3363 and 3364 may be wound around or unwound from the pitch driving pulley 3330 when the pitch driving pulley 3330 is rotated. For example, when the pitch driving pulley 3330 is rotated in one direction, one pitch wire may be wound around the pitch driving pulley 3330, and another pitch wire may be unwound from the pitch driving pulley 3330. For example, when the pitch driving pulley 3330 is rotated clockwise with respect to FIG. 28, the first pitch wire 3363 may be unwound from the pitch driving pulley 3330, and the second pitch wire 3364 may be wound around the pitch driving pulley 3330. In contrast, when the pitch driving pulley 3330 is rotated counterclockwise with respect to FIG. 28, the first pitch wire 3363 may be wound around the pitch driving pulley 3330, and the second pitch wire 3364 may be unwound from the pitch driving pulley 3330.

From another viewpoint, it may be stated that, when the pitch driving pulley 3330 is rotated, the pair of pitch wires 3363 and 3364 are moved in opposite directions with respect to the pitch driving pulley 3330.

As such, when the pitch driving pulley 3330 is rotated to move the pair of pitch wires 3363 and 3364 in different directions, the pulley connected to the pitch wires 3363 and 3364 on the end tool 1100 side is rotated in the corresponding direction. Accordingly, the pulley connected to the pitch wires 3363 and 3364 on the end tool 1100 side may implement the pitch rotation of the end tool 1100 while rotating in one direction.

The firing driving pulley 3340 may be a pulley associated with the movement of the operation member 1140 provided in the end tool 1100, and the firing wires 3365 and 3366 may be wires associated with the movement of the operation member 1140 provided in the end tool 1100. The firing driving pulley 3340 may be arranged in an area of the pulley frame 3310.

In other embodiments, the firing driving pulley 3340 may be arranged at a position which is not parallel to the yaw driving pulley 3320 and the pitch driving pulley 3330 of the pulley frame 3310. For example, the firing driving pulley 3340 may be arranged under the yaw driving pulley 3320 and the pitch driving pulley 3330. For example, as illustrated in FIG. 26, the yaw driving pulley 3320 and the pitch driving pulley 3330 may be arranged in parallel to each other on the pulley frame 3310, and the firing driving pulley 3340 may be arranged under the yaw driving pulley 3320 and the pitch driving pulley 3330.

In an embodiment, the firing wires 3365 and 3366 may be connected to a moving member (not shown) of the end tool 1100. In this case, the firing wires 3365 and 3366 may extend from the moving member toward the firing driving pulley 3340. In another embodiment, the firing wires 3365 and 3366 may be directly connected to the operation member 1140. In this case, the firing wires 3365 and 3366 may extend from the operation member 1140 toward the firing driving pulley 3340. In another embodiment, when the end tool 1100 is provided with a separate pulley associated with the translational motion of the operation member 1140, the firing wires 3365 and 3366 may be connected to the pulley, and the firing wires 3365 and 3366 may extend from the pulley toward the firing driving pulley 3340.

In an embodiment, the firing wires 3365 and 3366 may be provided as a pair. As described below, the firing wires 3365 and 3366 may be respectively connected to the left side and the right side of the firing driving pulley 3340.

In other embodiments, the firing wires 3365 and 3366 may include a first firing wire 3365 and a second firing wire 3366. The first firing wire 3365 may extend leftward from the firing driving pulley 3340 with respect to FIG. 28 and may be connected to the firing driving pulley 3340. The second firing wire 3366 may extend rightward from the firing driving pulley 3340 with respect to FIG. 28 and may be connected to the firing driving pulley 3340.

After the firing wires 3365 and 3366 are connected to the firing driving pulley 3340, the firing wires 3365 and 3366 may be wound around or unwound from the firing driving pulley 3340 when the firing driving pulley 3340 is rotated. For example, when the firing driving pulley 3340 is rotated in one direction, one of the firing wires 3365 and 3366 may be wound around the firing driving pulley 3340, and another of the firing wires 3365 and 3366 may be unwound from the firing driving pulley 3340. For example, when the firing driving pulley 3340 is rotated clockwise with respect to FIG. 28, the first firing wire 3365 may be unwound from the firing driving pulley 3340, and the second firing wire 3366 may be wound around the firing driving pulley 3340. In contrast, when the firing driving pulley 3340 is rotated counterclockwise with respect to FIG. 28, the first firing wire 3365 may be wound around the firing driving pulley 3340, and the second firing wire 3366 may be unwound from the firing driving pulley 3340.

From another viewpoint, it may be stated that, when the firing driving pulley 3340 is rotated, the pair of firing wires 3365 and 3366 are moved in opposite directions with respect to the firing driving pulley 3340.

As such, when the firing driving pulley 3340 is rotated to move the pair of firing wires 3365 and 3366 in different directions, the operation member 1140 provided in the end tool 1100 may be moved forward or backward accordingly. As an example, when the operation member 1140 is formed to be moved dependently by the movement of the moving member, the firing driving pulley 3340 may be rotated so that the pair of firing wires 3365 and 3366 may move the moving member forward or backward, and thus, the operation member 1140 may be moved forward or backward. As another example, when the operation member 1140 is formed to be connected to the firing wires 3365 and 3366 and directly moved forward or backward, the firing driving pulley 3340 may be rotated so that the pair of firing wires 3365 and 3366 may directly move the operation member 1140 forward or backward. However, the disclosure is not limited thereto, and when the end tool 1100 is provided with a separate pulley associated with the translational motion of the operation member 1140, the firing wires 3365 and 3366 may be connected to the pulley to rotate the pulley so that the operation member 1140 may be moved forward or backward.

In an embodiment, the power transmission part 3300 may further include at least one auxiliary pulley 3350. The auxiliary pulley 3350 may change the paths of wires entering the power transmission part 3300.

In an embodiment, the auxiliary pulley 3350 may include a first auxiliary pulley 3351 connected to the yaw wires 3361 and 3362 and the pitch wires 3363 and 3364 and changing the paths of the yaw wires 3361 and 3362 and the pitch wires 3363 and 3364. In some embodiments, the auxiliary pulley 3350 may include a second auxiliary pulley 3352 and a third auxiliary pulley 3353 connected to the firing wires 3365 and 3366 and changing the paths of the firing wires 3365 and 3366.

The first auxiliary pulley 3351 may be arranged in the power transmission part 3300 and may change the paths of the yaw wires 3361 and 3362 and the pitch wires 3363 and 3364 extending from the end tool 1100 to the power transmission part 3300 through a connection part 3400.

A first auxiliary pulley fixing part 3311 may be formed in the pulley frame 3310. The first auxiliary pulley fixing part 3311 may be a portion where the first auxiliary pulley 3351 is installed. For example, the first auxiliary pulley fixing part 3311 may be formed integrally with the pulley frame 3310. For example, the first auxiliary pulley fixing part 3311 may be formed as a separate member and may be coupled or assembled to the pulley frame 3310.

In an embodiment, the first auxiliary pulley fixing part 3311 may include at least one through hole, and the rotation shaft of the first auxiliary pulley 3351 may be arranged to pass through the through hole. From another viewpoint, the first auxiliary pulley 3351 may be arranged to overlap in parallel to the through hole formed in the first auxiliary pulley fixing part 3311, and the rotation shaft may be arranged to pass through both the first auxiliary pulley 3351 and the first auxiliary pulley fixing part 3311.

The first auxiliary pulley fixing part 3311 may be formed in a shape in which at least a portion of the first auxiliary pulley fixing part 3311 extends from the pulley frame 3310 toward the connection part 3400. For example, it may be stated that the first auxiliary pulley fixing part 3311 is formed to extend from a surface of the pulley frame 3310 toward the connection part 3400. In other embodiments, the first auxiliary pulley fixing part 3311 may be arranged at the center of the pulley frame 3310.

In this case, the yaw driving pulley 3320 and the pitch driving pulley 3330 may be respectively arranged on opposite sides of the first auxiliary pulley fixing part 3311. In other embodiments, the yaw driving pulley 3320 and the pitch driving pulley 3330 may be respectively arranged at positions symmetrical with each other with respect to the first auxiliary pulley fixing part 3311. From another viewpoint, the yaw wires 3361 and 3362 and the pitch wires 3363 and 3364 entering the first auxiliary pulley 3351 may be distributed to opposite sides while passing through the first auxiliary pulley fixing part 3311. Accordingly, it may be stated that the yaw wires 3361 and 3362 may extend toward the yaw driving pulley 3320, and the pitch wires 3363 and 3364 may extend toward the pitch driving pulley 3330.

Therefore, the yaw wires 3361 and 3362 may substantially vertically approach the yaw driving pulley 3320, and the pitch wires 3363 and 3364 may substantially vertically approach the pitch driving pulley 3330. For example, the yaw wires 3361 and 3362 may vertically approach the yaw driving pulley 3320, and the pitch wires 3363 and 3364 may vertically approach the pitch driving pulley 3330. From another viewpoint, it may be stated that the yaw wires 3361 and 3362 may approach the yaw driving pulley 3320 so as to form a tangent to the yaw driving pulley 3320, and the pitch wires 3363 and 3364 may approach the pitch driving pulley 3330 so as to form a tangent to the pitch driving pulley 3330.

The first auxiliary pulley 3351 may be arranged in the first auxiliary pulley fixing part 3311 and may change the paths of the yaw wires 3361 and 3362 and the pitch wires 3363 and 3364 extending to the power transmission part 3300.

For example, as illustrated in FIG. 28, the first auxiliary pulley 3351 may be arranged between the yaw driving pulley 3320 and the pitch driving pulley 3330.

A plurality of first auxiliary pulleys 3351 may be provided. The first auxiliary pulleys 3351 may be provided at least as many as the number of wires entering the power transmission part 3300.

As an example, the yaw wires 3361 and 3362 may be provided as a pair to enter the upper side and the lower side of the yaw driving pulley 3320, respectively. In some embodiments, the pitch wires 3363 and 3364 may be provided as a pair to enter the upper side and the lower side of the pitch driving pulley 3330, respectively. For example, when four wires entering the power transmission part 3300 are provided, four first auxiliary pulleys 3351 may be provided.

In other embodiments, the plurality of first auxiliary pulleys 3351 may be arranged side by side. For example, the plurality of first auxiliary pulleys 3351 may be arranged parallel to each other. This may be said to ensure that the wires going around the first auxiliary pulley 3351 extend to the yaw driving pulley 3320 and the pitch driving pulley 3330 so as to be parallel or substantially parallel to each other.

For example, the wires extending from the end tool 1100 to the power transmission part 3300 extend parallel to each other. Accordingly, because the plurality of first auxiliary pulleys 3351 are arranged parallel to each other, the wires going around the first auxiliary pulley 3351 may extend to the yaw driving pulley 3320 and/or the pitch driving pulley 3330 so as to be parallel or substantially parallel to each other.

In other embodiments, the first auxiliary pulley 3351 may be arranged so that the winding directions of the wires may be substantially perpendicular to each other in relation to the yaw driving pulley 3320 and the pitch driving pulley 3330, or may be perpendicular to each other. Referring to FIG. 28, the direction in which the wire is wound around the first auxiliary pulley 3351 and the direction in which the wire is wound around the yaw driving pulley 3320 and the pitch driving pulley 3330 may be perpendicular to each other. From another viewpoint, it may be stated that the first auxiliary pulley 3351 changes the traveling direction of the wire entering the first auxiliary pulley 3351 by 90°. From another viewpoint, it may be stated that the groove formed in the first auxiliary pulley 3351 and the grooves formed in the yaw driving pulley 3320 and the pitch driving pulley 3330 may be perpendicular to each other.

With this configuration, the paths of the wires entering the power transmission part 3300 may be changed through the first auxiliary pulley 3351, and the wires entering the power transmission part 3300 may vertically or substantially vertically approach toward the yaw driving pulley 3320 and the pitch driving pulley 3330.

The second auxiliary pulley 3352 may be arranged in the power transmission part 3300 and may change the paths of the firing wires 3365 and 3366 extending from the end tool 1100 to the power transmission part 3300 through the connection part 3400.

The third auxiliary pulley 3353 may be arranged in the power transmission part 3300 and may change the paths of the firing wires 3365 and 3366 extending from the second auxiliary pulley 3352.

Referring again to FIG. 26, the second auxiliary pulley 3352 may guide the firing wires 3365 and 3366 upward, which enter the power transmission part 3300 from the end tool 1100 through the connection part 3400. For example, the second auxiliary pulley 3352 may change the paths of the firing wires 3365 and 3366 entering the power transmission part 3300 to guide the firing wires 3365 and 3366 in the opposite direction to the direction in which the firing driving pulley 3340 is located. The third auxiliary pulley 3353 may change the paths of the firing wires 3365 and 3366 extending from the second auxiliary pulley 3352 to guide the firing wires 3365 and 3366 toward the firing driving pulley 3340.

Accordingly, the firing wires 3365 and 3366 may extend in a direction away from the firing driving pulley 3340 due to the second auxiliary pulley 3352 and then extend again toward the firing driving pulley 3340.

As described above, the firing driving pulley 3340 and the firing wires 3365 and 3366 may be associated with the movement of the operation member 1140 of the end tool 1100. For example, the firing wires 3365 and 3366 may be wound around the firing driving pulley 3340 more times than the yaw wires 3361 and 3362 or the pitch wires 3363 and 3364. This is related to the movement of the operation member 1140. For example, this is because the length by which the firing wires 3365 and 3366 are wound or unwound for the movement of the operation member 1140 is longer than the length by which the yaw wires 3361 and 3362 and the pitch wires 3363 and 3364 are wound or unwound for the yaw motion and the pitch motion of the end tool 1100.

At this time, because the groove formed in the firing driving pulley 3340 is formed in a thread root shape, the firing wires 3365 and 3366 may be moved in the axial direction of the firing driving pulley 3340 as the firing wires 3365 and 3366 are wound around the firing driving pulley 3340 a plurality of times. This means that an angle of the firing wires 3365 and 3366 extending from the third auxiliary pulley 3353 to the firing driving pulley 3340 increases (the firing wires 3365 and 3366 are connected obliquely). In this case, relatively strong stress may be applied to the firing wires 3365 and 3366.

Accordingly, the second auxiliary pulley 3352 may guide the firing wires 3365 and 3366 in a direction opposite to the firing driving pulley 3340, and the third auxiliary pulley 3353 may guide the firing wires 3365 and 3366 again toward the firing driving pulley 3340, so that the length of the firing wires 3365 and 3366 extending from the third auxiliary pulley 3353 to the firing driving pulley 3340 may be ensured. As a result, the change in the angle at which the firing wires 3365 and 3366 enter the firing driving pulley 3340 may be reduced.

A second auxiliary pulley fixing part 3312 may be formed in the pulley frame 3310. The second auxiliary pulley fixing part 3312 may be a portion where the third auxiliary pulley 3353 is installed. For example, the second auxiliary pulley fixing part 3312 may be formed integrally with the pulley frame 3310. For example, the second auxiliary pulley fixing part 3312 may be formed as a separate member and may be coupled or assembled to the pulley frame 3310.

In an embodiment, the second auxiliary pulley fixing part 3312 may include at least one through hole, and the rotation shaft of the third auxiliary pulley 3353 may be arranged to pass through the through hole. From another viewpoint, the third auxiliary pulley 3353 may be arranged to overlap the through hole formed in the second auxiliary pulley fixing part 3312 in parallel, and the rotation shaft may be arranged to pass through both the third auxiliary pulley 3353 and the second auxiliary pulley fixing part 3312.

The second auxiliary pulley fixing part 3312 may be formed in a shape which protrudes from the pulley frame 3310 toward the connection part 3400. For example, it can be said that the second auxiliary pulley 3352 fixing portion 3312 is formed to protrude from one side of the pulley frame 3310 toward the connection part 3400. In other embodiments, the second auxiliary pulley fixing part 3312 may be arranged over the pulley frame 3310. For example, the second auxiliary pulley fixing part 3312 may be arranged on the opposite side of the firing driving pulley 3340 with respect to the center of the pulley frame 3310.

In an embodiment, the second auxiliary pulley 3352 and the third auxiliary pulley 3353 may each be provided as a pair. For example, the second auxiliary pulley 3352 may include a pulley to which the first firing wire 3365 is connected and a pulley to which the second firing wire 3366 is connected. In some embodiments, the third auxiliary pulley 3353 may include a pulley to which the first firing wire 3365 is connected and a pulley to which the second firing wire 3366 is connected.

In other embodiments, two second auxiliary pulleys 3352 may be arranged parallel to each other. This may be said to ensure that the wires 3365 and 3366 going around the second auxiliary pulley 3352 extend to the third auxiliary pulley 3353 so as to be parallel or substantially parallel to each other.

For example, the wires 3365 and 3366 extending from the end tool 1100 to the power transmission part 3300 extend parallel to each other. Accordingly, because the two second auxiliary pulleys 3352 are arranged parallel to each other, the wires 3365 and 3366 going around the second auxiliary pulley 3352 may extend to the third auxiliary pulley 3353 so as to be parallel or substantially parallel to each other.

In other embodiments, two third auxiliary pulleys 3353 may be arranged parallel to each other. This may be said to ensure that the wires 3365 and 3366 going around the third auxiliary pulley 3353 extend to the firing driving pulley 3340 so as to be parallel or substantially parallel to each other.

For example, the wires 3365 and 3366 extending from the second auxiliary pulley 3352 to the third auxiliary pulley 3353 extend parallel to each other. Accordingly, because the two third auxiliary pulleys 3353 are arranged parallel to each other, the wires 3365 and 3366 going around the third auxiliary pulley 3353 may extend to the firing driving pulley 3340 so as to be parallel or substantially parallel to each other.

In other embodiments, the second auxiliary pulley 3352 and the third auxiliary pulley 3353 may be arranged so that the winding directions of the wires 3356 and 3366 may be substantially perpendicular to each other in relation to the firing driving pulley 3340, or may be perpendicular to each other. Referring to FIG. 26, the direction in which the wires 3365 and 3366 are wound around the second auxiliary pulley 3352 and the third auxiliary pulley 3353 may be perpendicular to the direction in which the wires 3365 and 3366 are wound around the firing driving pulley 3340. From another viewpoint, it may be stated that the groove formed in the first auxiliary pulley 3351 and the grooves formed in the yaw driving pulley 3320 and the pitch driving pulley 3330 are perpendicular to each other.

With this configuration, the paths of the wires 3365 and 3366 going around the second auxiliary pulley 3352 and the third auxiliary pulley 3353 may be changed, and the wires 3365 and 3366 going around the second auxiliary pulley 3352 and the third auxiliary pulley 3353 may vertically or substantially vertically approach toward the firing driving pulley 3340.

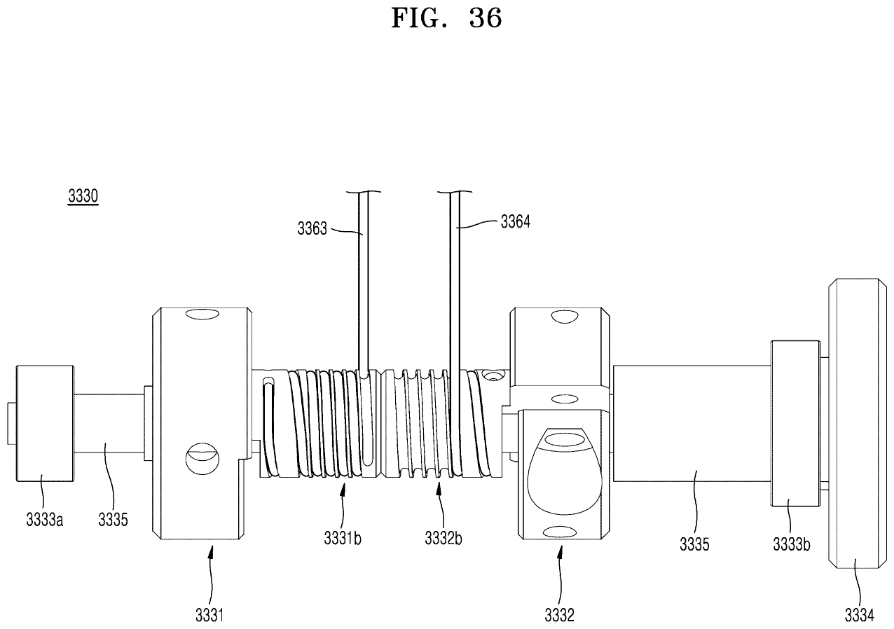

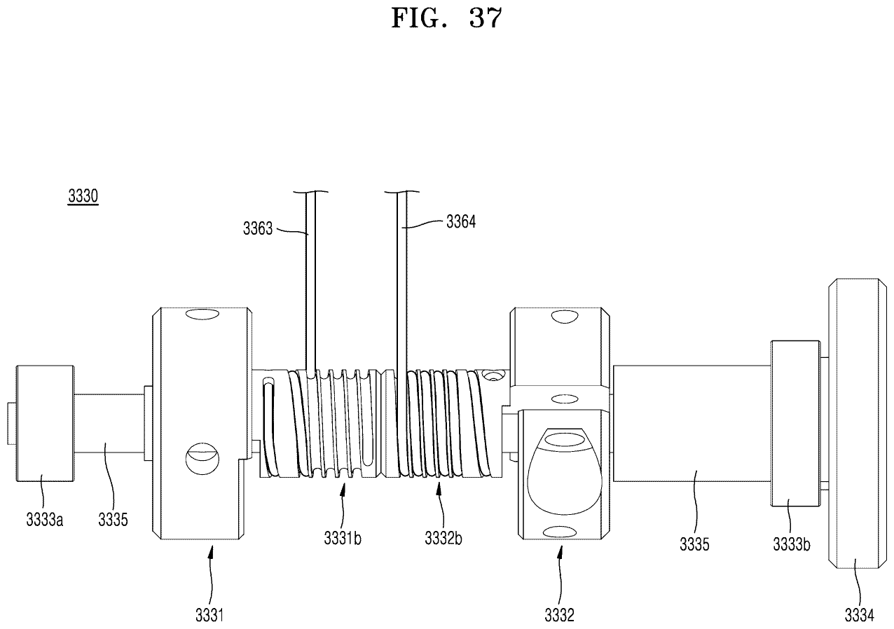

FIGS. 36 and 37 are diagrams for describing a process in which the pair of wires are wound around or unwound from the driving pulley of FIG. 26.

The driving pulley according to the second embodiment may include a driving pulley rotation shaft, a first part, a second part, a bearing, and a pulley plate.

For example, the pitch driving pulley 3330 may include a driving pulley rotation shaft 3335, a first part 3331, a second part 3332, a bearing 3333, and a pulley plate 3334.

The first part 3331 and the second part 3332 may be parts which the wires 3363 and 3364 are connected to and wound around, respectively, and the driving pulley body may be separated into two parts.

The first part 3331 and the second part 3332 may be coupled to the driving pulley rotation shaft 3335 and may be rotated together according to the rotation of the driving pulley rotation shaft 3335.

The first part 3331 may form a groove around which the first wire 3363 is wound, and the second part 3332 may form a groove around which the second wire 3364 is wound.

The wires wound around the first part 3331 and the second part 3332 may be wound in opposite directions. For example, the first wire 3363 may be wound clockwise around the first part 3331, and the second wire 3364 may be wound counterclockwise around the second part 3332.

Accordingly, when the pitch driving pulley 3330 is rotated in one direction, one pitch wire may be wound around the pitch driving pulley 3330, and another pitch wire may be unwound from the pitch driving pulley 3330.

For example, in the pitch driving pulley 3330, the pitch wire 3363 may be connected to the first part 3331, and the pitch wire 3364 may be connected to the second part 3332. For example, with respect to FIG. 26, the pitch wire 3363 may be wound clockwise around the first part 3331, and the pitch wire 3364 may be wound counterclockwise around the second part 3332. Accordingly, when the pitch driving pulley 3330 is rotated clockwise, the pitch wire 3363 may be unwound from the first part 3331, and the pitch wire 3364 may be wound around the second part 3332.

FIG. 36 illustrates a state where the pitch wire 3363 is wound maximally around the first part 3331, and the pitch wire 3364 is wound minimally around the second part 3332.

FIG. 37 illustrates a state where the pitch wire 3364 is wound maximally around the second part 3332, and the pitch wire 3363 is wound minimally around the first part 3331.

As described above, when the pitch driving pulley 3330 is rotated clockwise, the pitch wire 3363 is unwound from the first part 3331, and the pitch wire 3364 is wound around the second part 3332, and thus, the positions of the grooves around which the wires are wound may be changed.

Accordingly, when rotated clockwise, the pitch driving pulley 3330 in the state illustrated in FIG. 36 may become the pitch driving pulley 3330 in the state illustrated in FIG. 37.

In some embodiments, a bearing 3333b may be arranged at an end portion of the driving pulley rotation shaft 3335, and a bearing 3333a may be arranged at another end of the driving pulley rotation shaft 3335. The bearing 3333 may support the driving pulley shaft 3335 to facilitate the axial rotation of the pitch driving pulley 3330 in the pulley frame 3310.

The pulley plate 3334 may be coupled to an end portion of the driving pulley rotation shaft 3335.

The pulley plate 3334 may further include an insertion hole 3334a (see, FIG. 27) which is recessed inward. The insertion hole 3334a may be fastened to a motor plate provided on a driving motor of another device. For example, a protrusion protruding from the motor plate may be inserted into the insertion hole 3334a of the pulley plate 3334 so that the motor plate and the pulley plate 3334 may be fastened to each other. Accordingly, a rotation force of the driving motor may be transmitted to the pitch driving pulley 3330 through the pulley plate 3334. For example, the pitch driving pulley 3330 may be axially rotated by receiving the rotation force applied to the pulley plate 3334. However, the concept of the disclosure is not limited thereto, and a protrusion protruding outward may be formed on the pulley plate 3334 and inserted into the insertion hole of the motor plate so that the pulley plate 3334 and the motor plate may be fastened to each other.

The structure of the driving pulley has been described by taking the pitch driving pulley 3330 as an example. Because the yaw driving pulley 3320 and the firing driving pulley 3340 are substantially identical to the pitch driving pulley 3330 in a corresponding range, a detailed description thereof is omitted.

Hereinafter, a power transmission part according to a third embodiment is described.

The power transmission part according to the third embodiment differs from the power transmission part according to the second embodiment in terms of the arrangement of the auxiliary pulley. A configuration which is different from the second embodiment is described in detail below.

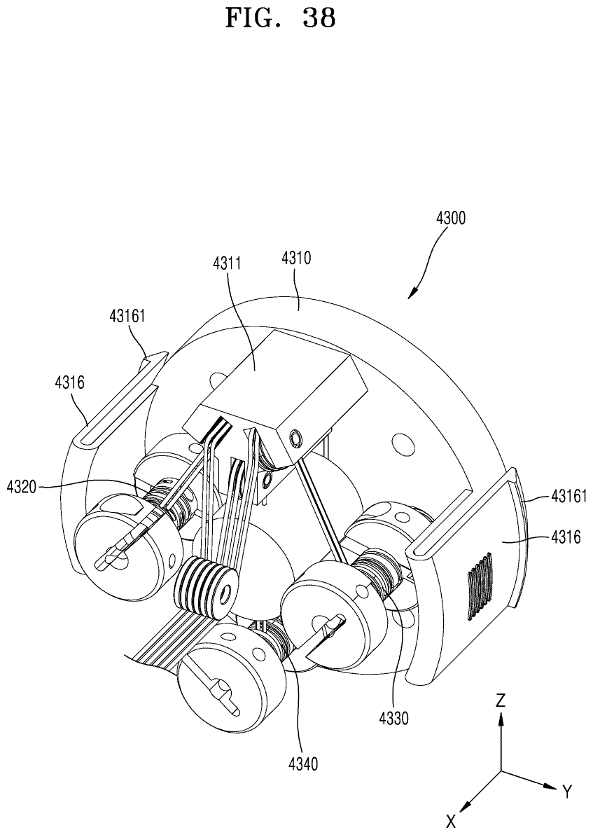

FIG. 38 is a diagram for describing the internal structure of the power transmission part according to the third embodiment.

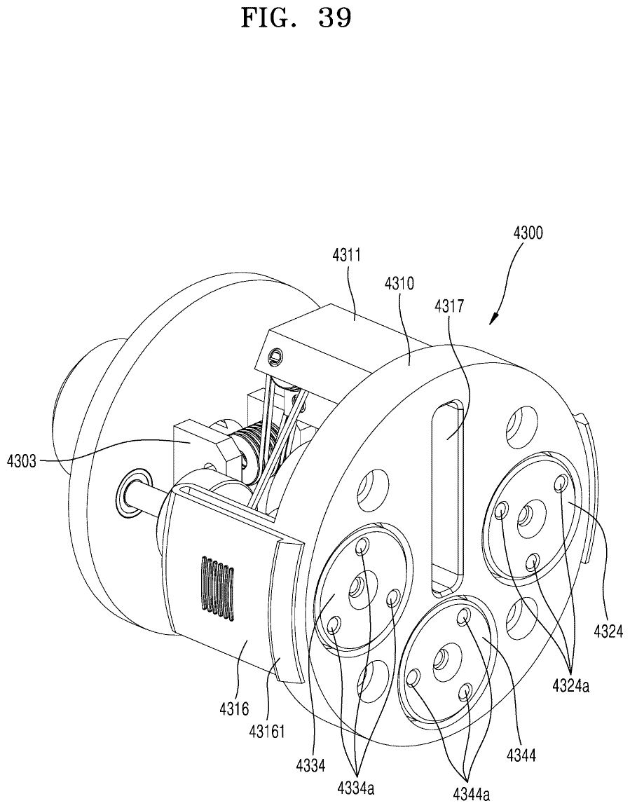

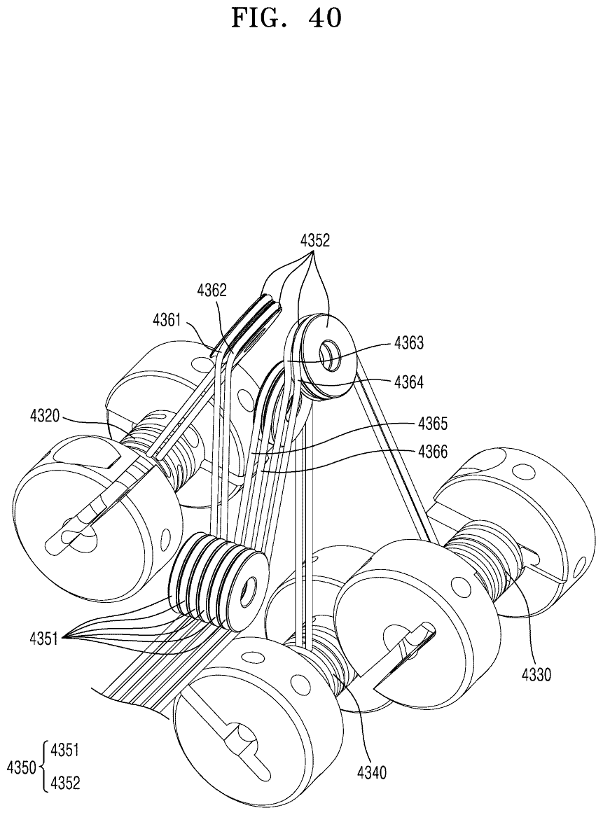

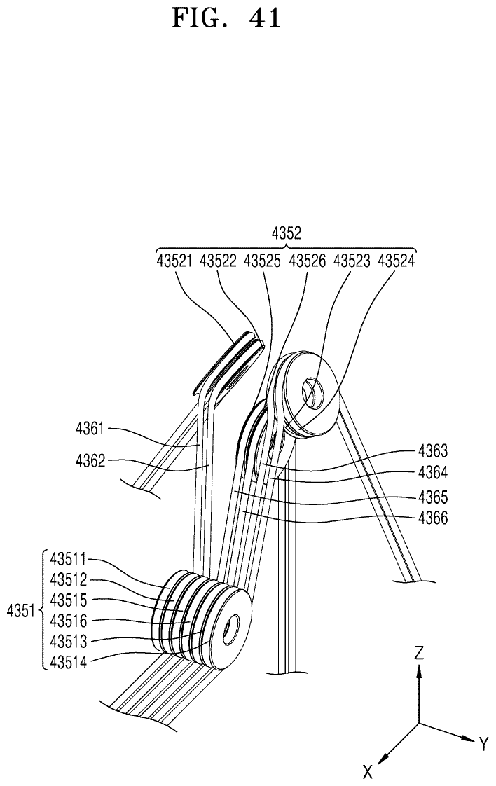

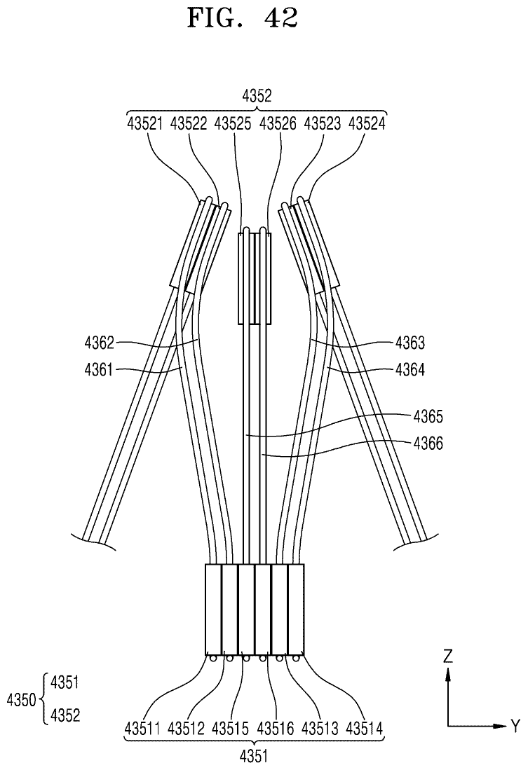

FIG. 39 is a diagram illustrating the power transmission part according to the third embodiment, when viewed from behind. FIG. 40 is a diagram for describing an arrangement of a pulley and a wire in the power transmission part of FIG. 38. FIG. 41 is a perspective view illustrating an auxiliary pulley and a wire in the power transmission part of FIG. 40. FIG. 42 is a diagram illustrating the auxiliary pulley and the wire of FIG. 41, when viewed from the front.

Referring to FIGS. 38 to 42, the power transmission part according to the third embodiment may include a pulley frame 4310, a yaw driving pulley 4320, a pitch driving pulley 4330, and a firing driving pulley 4340. In some embodiments, the power transmission part 4300 may include at least one yaw wire 4361 and 4362, at least one pitch wire 4363 and 4364, and firing wires 4365 and 4366.

In some embodiments, the driving pulley may include a driving pulley rotation shaft, a first part, a second part, a bearing, and a pulley plate. In the present embodiment, because the parts related to the driving pulley are substantially identical to the driving pulley rotation shaft 3335, the first part 3331, the second part 3332, the bearing 3333, and the pulley plate 3334 described with reference to FIGS. 36 and 37, a detailed description thereof is omitted herein.

The power transmission part 4300 according to the third embodiment may further include at least one auxiliary pulley 4350. The auxiliary pulley 4350 may change the paths of wires entering the power transmission part 4300.

In other words, the power transmission part 4300 may change the path of the wire extending from the inside of the shaft to the power transmission part 4300 through the auxiliary pulley 4350, so as to guide the wire to be connected to the driving pulley.

In an embodiment, the auxiliary pulley 4350 may include a first auxiliary pulley 4351 connected to the yaw wires 4361 and 4362, the pitch wires 4363 and 4364, and the firing wires 4365 and 4366 and changing the paths of the yaw wires 4361 and 4362, the pitch wires 4363 and 4364, and the firing wires 4365 and 4366.

In some embodiments, the auxiliary pulley 4350 may further include a second auxiliary pulley 4352 connected to the respective wires through the first auxiliary pulley 4351 and changing the paths of the respective wires.

For example, the first auxiliary pulley 4351 may include first yaw wire auxiliary pulleys 43511 and 43512, first pitch wire auxiliary pulleys 43513 and 43514, and first firing wire auxiliary pulleys 43515 and 43516.

The second auxiliary pulley 4352 may include second yaw wire auxiliary pulleys 43521 and 43522, second pitch wire auxiliary pulleys 43523 and 43524, and second firing wire auxiliary pulleys 43525 and 43526.

The yaw wires 4361 and 4362 may be connected to the second yaw wire auxiliary pulleys 43521 and 43522 through the first yaw wire auxiliary pulleys 43511 and 43512.

The pitch wires 4363 and 4364 may be connected to the second pitch wire auxiliary pulleys 43523 and 43524 through the first pitch wire auxiliary pulleys 43513 and 43514.

The firing wires 4365 and 4366 may be connected to the second firing wire auxiliary pulleys 43525 and 43526 through the first firing wire auxiliary pulleys 43515 and 43516.

The first auxiliary pulley 4351 may be arranged so that a rotation shaft of the first auxiliary pulley 4351 is perpendicular to an extension shaft of the shaft.

From another viewpoint, the rotation shaft of the first auxiliary pulley 4351 may be arranged perpendicular to the rotation shafts of the yaw driving pulley 4320 and the pitch driving pulley 4330.

From another viewpoint, the rotation plane on which the first auxiliary pulley 4351 is rotated may be arranged between the yaw driving pulley 4320 and the pitch driving pulley 4330.

The first auxiliary pulley 4351 may guide the wire upward, which enters the power transmission part 4300 from the end tool (not shown, see 1100) through the connection part (not shown, see 3400).

For example, the first firing wire auxiliary pulleys 43515 and 43516 may change the paths of the firing wires 4365 and 4366 entering the power transmission part 4300 to guide the firing wires 4365 and 4366 in the opposite direction to the direction in which the firing driving pulley 4340 is located. The second firing wire auxiliary pulleys 43525 and 43526 may change the paths of the firing wires 4365 and 4366 extending from the first firing wire auxiliary pulleys 43515 and 43516 to guide the firing wires 4365 and 4366 toward the firing driving pulley 4340.

Accordingly, the firing wires 4365 and 4366 may extend in a direction away from the firing driving pulley 4340 due to the first firing wire auxiliary pulleys 43515 and 43516 and then extend again toward the firing driving pulley 4340.

The first yaw wire auxiliary pulleys 43511 and 43512 and the first pitch wire auxiliary pulleys 43513 and 43514 may also be arranged parallel to the first firing auxiliary pulleys.

For example, the first yaw wire auxiliary pulleys 43511 and 43512 may guide the yaw wires 4361 and 4362 upward. The first pitch wire auxiliary pulleys 43513 and 43514 may guide the pitch wires 4363 and 4364 upward.

The second yaw wire auxiliary pulleys 43521 and 43522 and the second pitch wire auxiliary pulleys 43523 and 43524 may be spaced apart from the second firing wire auxiliary pulleys 43525 and 43526.

The second yaw wire auxiliary pulleys 43521 and 43522 may not be parallel to the second firing wire auxiliary pulleys 43525 and 43526. For example, the second yaw wire auxiliary pulleys 43521 and 43522 may be inclined with respect to the second firing wire auxiliary pulleys 43525 and 43526.

From another viewpoint, the rotation shafts of the second yaw wire auxiliary pulleys 43521 and 43522 may not be parallel to the rotation shafts of the second firing wire auxiliary pulleys 43525 and 43526.

For example, the rotation plane on which the second yaw wire auxiliary pulleys 43521 and 43522 are rotated may include a rotation shaft of the yaw driving pulley 4320. Accordingly, the yaw wires 4361 and 4362 passing through the second yaw wire auxiliary pulleys 43521 and 43522 may be connected perpendicular to the yaw driving pulley 4320.

Similarly, the second pitch wire auxiliary pulleys 43523 and 43524 may not be parallel to the second firing wire auxiliary pulleys 43525 and 43526. For example, the second pitch wire auxiliary pulleys 43523 and 43524 may be inclined with respect to the second firing wire auxiliary pulleys 43525 and 43526.

From another viewpoint, the rotation shafts of the second pitch wire auxiliary pulleys 43523 and 43524 may not be parallel to the rotation shafts of the second firing wire auxiliary pulleys 43525 and 43526.

For example, the rotation plane on which the second pitch wire auxiliary pulleys 43523 and 43524 are rotated may include a rotation shaft of the pitch driving pulley 4330. Accordingly, the pitch wires 4363 and 4364 passing through the second pitch wire auxiliary pulleys 43523 and 43524 may be connected perpendicular to the pitch driving pulley 4330.

In some embodiments, the pulley frame 4310 may include an auxiliary pulley fixing part 4311 which accommodates at least a portion of the second auxiliary pulley 4352. The rotation shaft of the second auxiliary pulley 4352 may be fixed to the auxiliary pulley fixing part 4311, and the second auxiliary pulley 4352 may be rotated around the rotation shaft.

The second auxiliary pulley 4352 may be arranged at the upper end portion of the pulley frame 4310 so that the wires passing through the first auxiliary pulley 4351 are respectively distributed to the driving pulleys. In some embodiments, by arranging the second auxiliary pulley away from each driving pulley, the length of each wire extending from the second auxiliary pulley to each driving pulley may be ensured. As a result, the change in the angle at which each wire enters the driving pulley may be reduced.

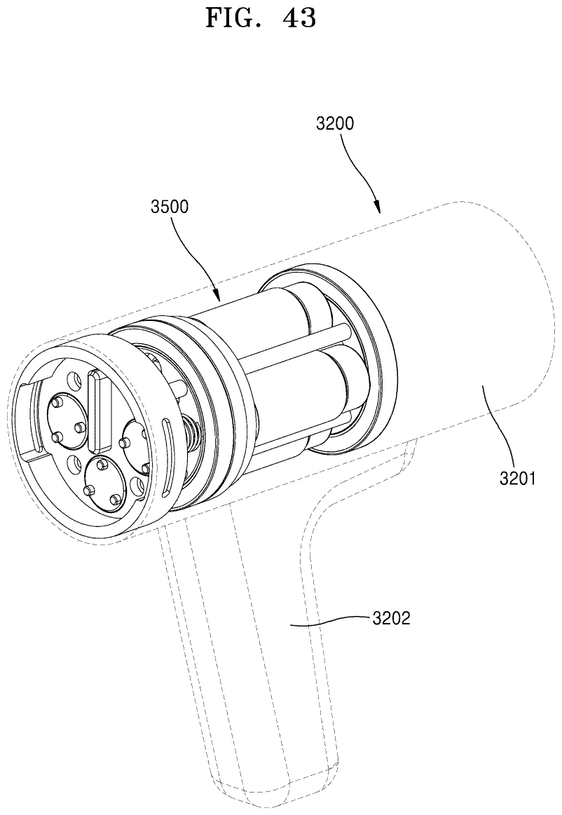

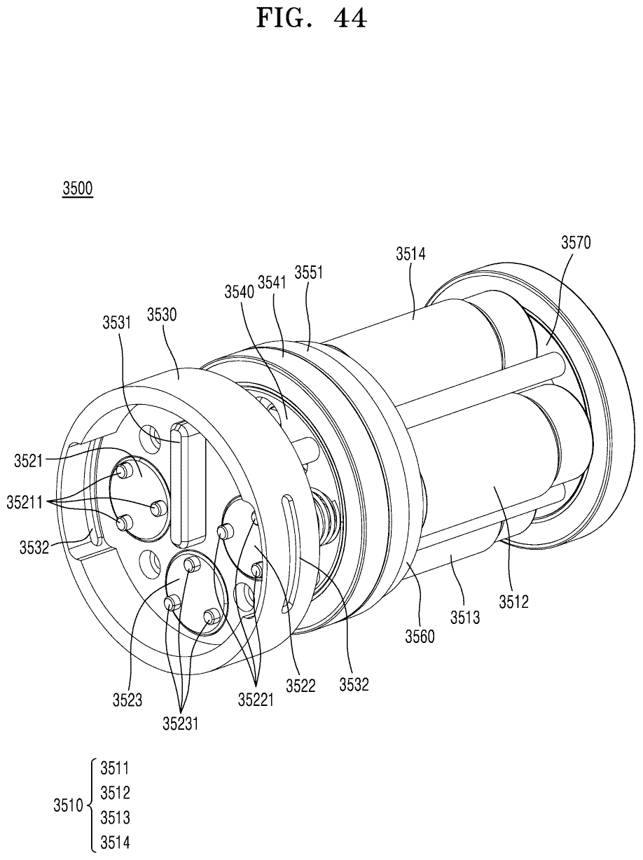

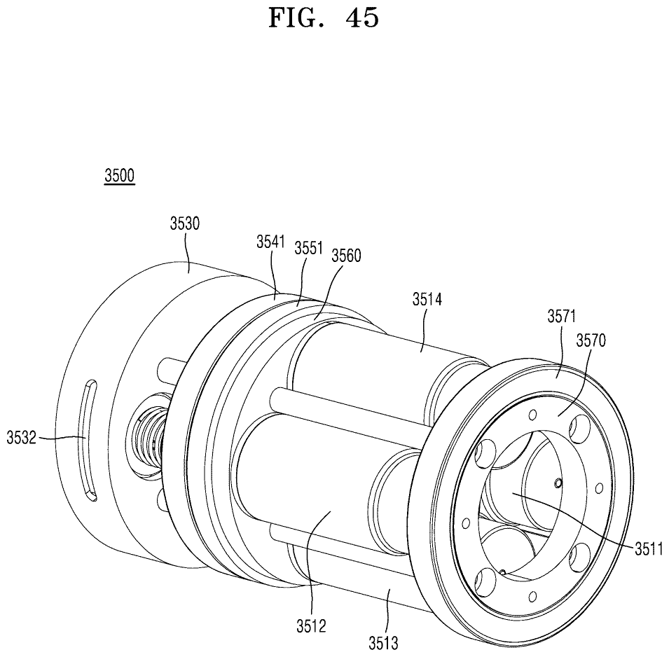

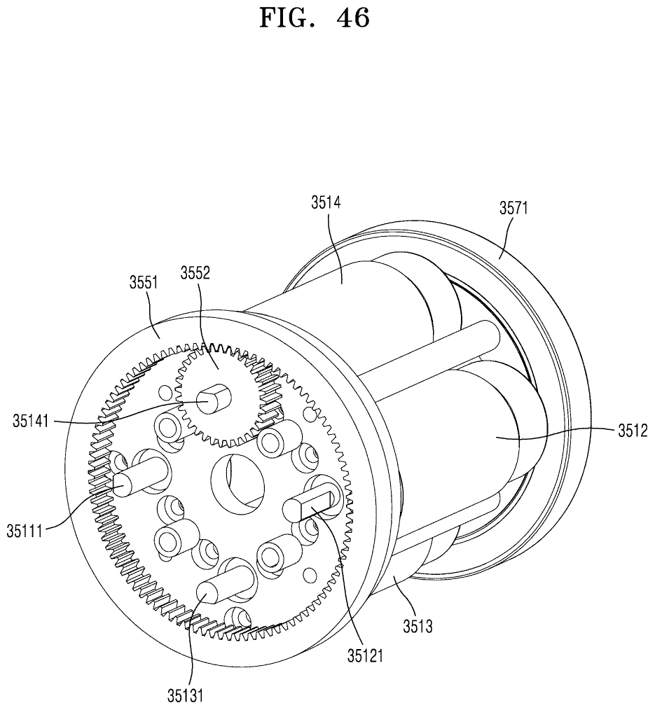

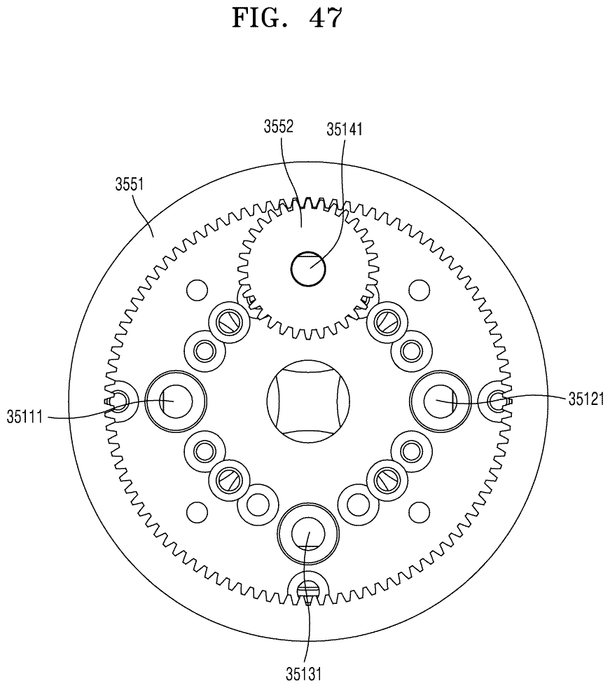

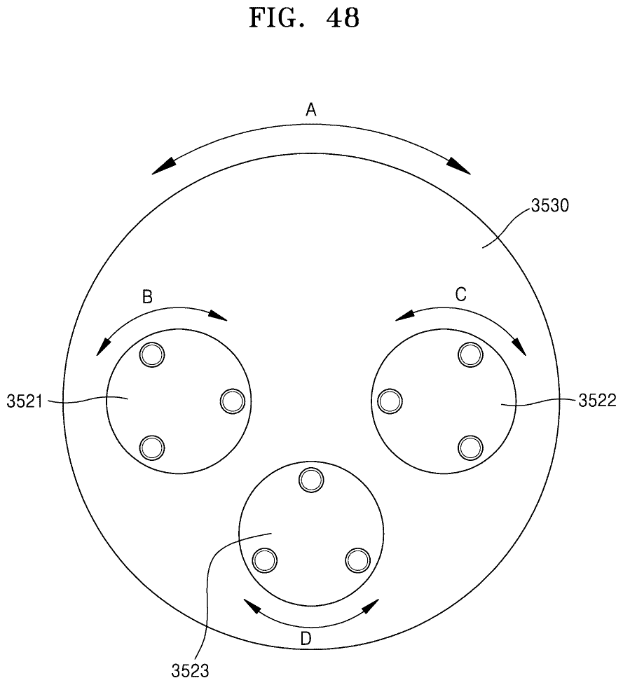

FIG. 43 is a diagram illustrating a manipulation part and a power generation part of a surgical instrument according to the second embodiment, and FIG. 44 is a perspective view illustrating the power generation part of FIG. 43. FIG. 45 is a diagram illustrating the power generation part of FIG. 44, when viewed from behind, and FIG. 46 is a diagram for describing a gear structure of the power generation part of FIG. 44. FIG. 47 is a diagram illustrating the gear structure of FIG. 46, when viewed from the front, and FIG. 48 is a diagram for describing the rotation of the power generation part of FIG. 44.

Referring to FIGS. 43 to 48, a surgical instrument according to an embodiment may include a power generation part 3500 which generates power for controlling an end tool 1100.

The power generation part 3500 may be arranged so that at least a portion of the power generation part 3500 is accommodated in a housing 3201 of a manipulation part 3200. The housing 3201 may refer to a configuration which forms the outer shape of the manipulation part 3200. For a driving module of a surgical instrument, the housing 3201 may refer to a configuration which forms the outer shape of the module body.

When a user manipulates the manipulation part 3200, the power generation part 3500 may generate power for controlling the end tool 1100, based on the manipulation.

The power generation part 3500 may include a motor pack 3510 including at least one motor.

The motor pack 3510 may perform a roll rotation with respect to the extension direction of the connection part 3400.

The roll motion used in the disclosure is defined as follows.

The roll motion refers to a motion in which the end tool 1100, the connection part 3400, the motor pack 3510, and the like constituting the surgical instrument 3000 are rotated with respect to the extension direction of the connection part 3400. In other words, the roll motion refers to a motion of rotation without bending in the Y-axis direction of FIG. 2 or the Z-axis direction of FIG. 2 with respect to the extension direction (the X-axis direction of FIG. 2) of the connection part 3400.

Referring again to FIG. 43, at least a portion of the power generation part 3500 may be accommodated in the housing 3201 of the manipulation part 3200. In this case, the motor pack 3510 may be accommodated in the housing 3201 of the manipulation part 3200. The expression that “the motor pack 3510 performs a roll rotation may mean rotating along the inner circumferential surface of the housing 3201 in the housing 3201. From another viewpoint, when the user holds a handle 3202 of the manipulation part 3200 and performs a manipulation for the roll rotation of the motor pack 3510, the motor pack 3510 may be rotated with respect to the extension direction of the connection part 3400 in the housing 3201 in a state where the housing 3201 and the handle 3202 of the manipulation part 3200 are fixed in place. From another viewpoint, it may be stated that, when the user performs a manipulation for the roll motion while holding the connection part 3400, the housing 3201 and the handle 3202 are rotated.

The motor pack 3510 may include at least one motor. For example, the motor pack 3510 may include at least one motor which generates power for driving the end tool 1100, based on a signal input to the manipulation part 3200.

In other embodiments, the motor pack 3510 may further include a roll driving motor 3514.

The motor pack 3510 may include a yaw driving motor 3511. The yaw driving motor 3511 may generate power for the yaw rotation of the end tool 1100. For example, when the user manipulates the manipulation part 3200 for the yaw rotation of the end tool 1100, the yaw driving motor 3511 may generate a driving force for the yaw rotation of the end tool 1100.

The driving force generated by the yaw driving motor 3511 may be transmitted to the power transmission part 3300 to rotate the yaw driving pulley 3320. As the yaw wires 3361 and 3362 are moved by the rotation of the yaw driving pulley 3320, the end tool 1100 may perform a yaw rotation.

The yaw driving motor 3511 may include a yaw motor rotation shaft 35111 formed to extend in one direction. The yaw motor rotation shaft 35111 may be a portion which is rotated when the yaw driving motor 3511 is driven. For example, the yaw motor rotation shaft 35111 may be formed to extend from the main body of the yaw driving motor 3511 toward the power transmission part 3300. As described below, a yaw motor plate 3521 may be arranged at an end of the yaw motor rotation shaft 35111. When the yaw motor rotation shaft 35111 is rotated, the yaw motor plate 3521 may be rotated together. The yaw motor plate 3521 may be connected to the yaw driving pulley 3320. As the yaw motor plate 3521 is rotated, the driving force may be transmitted to the yaw driving pulley 3320.

The motor pack 3510 may include a pitch driving motor 3512. The pitch driving motor 3512 may generate power for the pitch rotation of the end tool 1100. For example, when the user manipulates the manipulation part 3200 for the pitch rotation of the end tool 1100, the pitch driving motor 3512 may generate a driving force for the pitch rotation of the end tool 1100.

The driving force generated by the pitch driving motor 3512 may be transmitted to the power transmission part 3300 to rotate the pitch driving pulley 3330. As the pitch wires 3363 and 3364 are moved by the rotation of the pitch driving pulley 3330, the end tool 1100 may perform a pitch rotation.

The pitch driving motor 3512 may include a pitch motor rotation shaft 35121 formed to extend in one direction. The pitch motor rotation shaft 35121 may be a portion which is rotated when the pitch driving motor 3512 is driven. For example, the pitch motor rotation shaft 35121 may be formed to extend from the main body of the pitch driving motor 3512 toward the power transmission part 3300. As described below, a pitch motor plate 3522 may be arranged at an end of the pitch motor rotation shaft 35121. When the pitch motor rotation shaft 35121 is rotated, the pitch motor plate 3522 may be rotated together. The pitch motor plate 3522 may be connected to the pitch driving pulley 3330. As the pitch motor plate 3522 is rotated, the driving force may be transmitted to the pitch driving pulley 3330.

The power generation part 3500 may include a roll driving motor 3514 which generates power for the roll rotation of the motor pack 3510. For example, when the user manipulates the manipulation part 3200 for the rotation of the motor pack 3510, the roll driving motor 3514 may generate a driving force for the roll rotation of the motor pack 3510.

In an embodiment, the roll driving motor 3514 may be provided in the motor pack 3510. For example, the roll driving motor 3514 may be provided in the motor back 3510 together with the yaw driving motor 3511 and the pitch driving motor 3512.

The roll driving motor 3514 may be driven by the manipulation of the user to generate a driving force for the rotation of the motor pack 3510. In this case, the roll driving motor 3514 may be moved together with the motor pack 3510. For example, when the roll rotation of the motor pack 3510 is performed by the roll driving motor 3514, the roll driving motor 3514 may be rotated together with the motor pack 3510 as a component provided in the motor pack 3510.

In another embodiment, the roll driving motor 3514 may not be provided in the motor pack 3510. For example, the roll driving motor 3514 may be arranged outside the motor pack 3510.

The roll driving motor 3514 may be driven by the manipulation of the user to generate a driving force for the rotation of the motor pack 3510. In this case, the roll driving motor 3514 may be moved independently of the motor pack 3510. For example, unlike as described below, the roll rotation of the motor pack 3510 may be performed by the roll driving motor 3514, but the roll driving motor 3514 may not be rotated together with the motor pack 3510. For example, the yaw driving motor 3511, the pitch driving motor 3512, and the firing driving motor 3513 may be rotated together, but the roll driving motor 3514 may not be rotated together.

Hereinafter, for convenience of explanation, an example in which the roll driving motor 3514 is provided in the motor pack 3510 is described. However, it will be understood from the following description by those of ordinary skill in the art that the roll driving motor 3514 may not be provided in the motor pack 3510. In some embodiments, it will be understood by those of ordinary skill in the art that, when the roll driving motor 3514 is not provided in the motor pack 3510, the following description may be appropriately modified and applied.

The motor pack 3510 may include a firing driving motor 3513. The firing driving motor 3513 may generate power for the linear motion of the operation member 1140 of the end tool 1100. For example, in case the user manipulates the manipulation part 3200 for the linear motion of the operation member 1140 of the end tool 1100, the firing driving motor 3513 may generate a driving force for the linear motion of the operation member 1140.

The firing driving motor 3513 may include a firing motor rotation shaft 35131 formed to extend in one direction. The firing motor rotation shaft 35131 may be a portion which is rotated when the firing driving motor 3513 is driven. For example, the firing motor rotation shaft 35131 may be formed to extend from the main body of the firing driving motor 3513 toward the power transmission part 3300. As described below, a firing motor plate 3523 may be arranged at an end of the firing motor rotation shaft 35131. When the firing motor rotation shaft 35131 is rotated, the firing motor plate 3523 may be rotated together. The firing motor plate 3523 may be connected to the firing driving pulley 3340. As the firing motor plate 3523 is rotated, the driving force may be transmitted to the firing driving pulley 3340.

The motor pack 3510 may include a base plate 3560. The base plate 3560 may be arranged in front of the yaw driving motor 3511, the pitch driving motor 3512, the roll driving motor 3514, and the firing driving motor 3513. The base plate 3560 may be connected to the roll driving motor 3514, the yaw driving motor 3511, the pitch driving motor 3512, and the firing driving motor 3513. From another viewpoint, it may be stated that the roll driving motor 3514, the yaw driving motor 3511, the pitch driving motor 3512, and the firing driving motor 3513 are connected to the base plate 3560. From another viewpoint, it may be stated that the base plate 3560 connects the roll driving motor 3514, the yaw driving motor 3511, the pitch driving motor 3512, and the firing driving motor 3513, so that the roll driving motor 3514, the yaw driving motor 3511, the pitch driving motor 3512, and the firing driving motor 3513 are moved or rotated as one body.

Accordingly, when the base plate 3560 is rotated by the driving force of the roll driving motor 3514, the roll driving motor 3514, the yaw driving motor 3511, the pitch driving motor 3512, and the firing driving motor 3513, which are connected to the base plate 3560, may be rotated simultaneously. For example, when the roll driving motor 3514 is driven, the base plate 3560 may be rotated, and when the base plate 3560 is rotated, the roll driving motor 3514, the yaw driving motor 3511, the pitch driving motor 3512, and the firing driving motor 3513, which are connected to the base plate 3560, may be rotated together with the base plate 3560. Because the base plate 3560 is rotated with respect to the extension direction of the connection part 3400, the motor pack 3510 including the base plate 3560, the roll driving motor 3514, the yaw driving motor 3511, the pitch driving motor 3512, and the firing driving motor 3513 may perform a roll rotation with respect to the extension direction of the connection part 3400.

In an embodiment, the roll driving motor 3514, the yaw driving motor 3511, the pitch driving motor 3512, and the firing driving motor 3513 may be arranged parallel to each other. In some embodiments, the roll driving motor 3514, the yaw driving motor 3511, the pitch driving motor 3512, and the firing driving motor 3513 may be arranged to form a circular pattern.

As described above, the motor pack 3510 may perform a roll rotation in the housing 3201 of the manipulation part 3200. In this case, because the motor pack 3510 includes a plurality of motors, the roll driving motor 3514, the yaw driving motor 3511, the pitch driving motor 3512, and the firing driving motor 3513 may be arranged to form a circular pattern, and thus, the diameter of the space occupied by the motor pack 3510 when rotated may be minimized. For example, because the roll driving motor 3514, the yaw driving motor 3511, the pitch driving motor 3512, and the firing driving motor 3513 are arranged to form a circular pattern, the inner diameter of the housing 3201 required for the rotation of the motor pack 3510 may be designed to be smaller, which may contribute to the miniaturization and weight reduction of the surgical instrument 3000.

In some embodiments, the expression that the roll driving motor 3514, the yaw driving motor 3511, the pitch driving motor 3512, and the firing driving motor 3513 are arranged to form a circular pattern does not mean that the roll driving motor 3514, the yaw driving motor 3511, the pitch driving motor 3512, and the firing driving motor 3513 are arranged at equal intervals, and it may be sufficient as long as the outer circumferential surfaces of the roll driving motor 3514, the yaw driving motor 3511, the pitch driving motor 3512, and the firing driving motor 3513 are arranged within a circle.

In an embodiment, the roll driving motor 3514, the yaw driving motor 3511, the pitch driving motor 3512, and the firing driving motor 3513 may be provided to having different performances. For example, the roll driving motor 3514, the yaw driving motor 3511, the pitch driving motor 3512, and the firing driving motor 3513 may have different magnitudes of driving forces which have to be generated so as to perform the respective functions. To this end, the roll driving motor 3514, the yaw driving motor 3511, the pitch driving motor 3512, and the firing driving motor 3513 may have different outputs or different sizes as necessary.

At least one through hole may be formed in the base plate 3560. For example, as many through holes as at least the number of motors included in the motor pack 3510 may be formed in the base plate 3560. The through hole is a portion through which the rotation shafts of the respective motors pass.

For example, the yaw motor rotation shaft 35111 may be formed to extend from the main body of the yaw driving motor 3511 and extend to pass through the base plate 3560 (through the through hole). In some embodiments, the pitch motor rotation shaft 35121 may be formed to extend from the main body of the pitch driving motor 3512 and extend to pass through the base plate 3560 (through the through hole). In some embodiments, the firing motor rotation shaft 35131 may be formed to extend from the main body of the firing driving motor 3513 and extend to pass through the base plate 3560 (through the through hole).

In some embodiments, the roll driving motor 3514 may include a roll motor rotation shaft 35141 formed to extend in one direction. The roll motor rotation shaft 35141 may be a portion which is rotated when the roll driving motor 3514 is driven. For example, the roll motor rotation shaft 35141 may be formed to extend forward from the main body of the roll driving motor 3514. For example, the roll motor rotation shaft 35141 may be formed to extend forward from the main body of the roll driving motor 3514 and extend to pass through the base plate 3560.

Hereinafter, the principle of rotation of the motor pack 3510 is described in detail.

Referring to FIGS. 46 and 47, the power generation part 3500 may include a first gear 3551 formed in a circular shape and a second gear 3552 engaged with the first gear 3551.

The first gear 3551 may be formed in the shape of a hollow circle, and gear teeth may be formed on the inner circumferential surface of the circle. For example, the first gear 3551 may be a type of ring gear having gear teeth formed on the inner circumferential surface.

The second gear 3552 is a gear with gear teeth formed on the outer circumferential surface and may be engaged with the first gear 3551. The second gear 3552 may be arranged in the roll motor rotation shaft 35141. For example, the roll motor rotation shaft 35141 may be formed to extend forward from the main body of the roll driving motor 3514 to pass through the base plate 3560, and the second gear 3552 may be arranged in the extension portion of the roll motor rotation shaft 35141. At this time, the second gear 3552 may be coupled to the roll motor rotation shaft 35141. When the roll motor rotation shaft 35141 is rotated, the second gear 3552 may be rotated together.

The first gear 3551 may be arranged in front of the base plate 3560. In some embodiments, the first gear 3551 may be fixed to the inner circumferential surface of the housing 3201. Accordingly, when the roll driving motor 3514 is driven, the motor pack 3510 may perform a roll rotation in the housing 3201.

For example, when the roll driving motor 3514 is driven, the roll motor rotation shaft 35141 may be rotated, and the second gear 3552 arranged in the roll motor rotation shaft 35141 may be rotated together. At this time, when the second gear 3552 is rotated, the first gear 3551 engaged with the second gear 3552 is fixed to the inner circumferential surface of the housing 3201, and thus, the second gear 3552 may be moved along the gear teeth of the first gear 3551. For example, when the roll driving motor 3514 is driven, the first gear 3551 and the second gear 3552 may be relatively rotated. At this time, because the first gear 3551 is fixed to the housing 3201, the second gear 3552 may be relatively moved along the first gear 3551. In some embodiments, the second gear 3552 may be connected to the roll motor rotation shaft 35141, the roll driving motor 3514 may be connected to the base plate 3560, and the base plate 3560 may be connected to the yaw driving motor 3511 and the pitch driving motor 3512. Accordingly, the motor pack 3510 may be rotated relative to the housing 3201 by the motions of the first gear 3551 and the second gear 3552. From another viewpoint, the motor pack 3510 may be rotated independently of the movement of the housing 3201.

For example, the base plate 3560 may be rotated relative to the housing 3201. For example, the second gear 3552 is connected to the base plate 3560 by the roll motor rotation shaft 35141, and thus, when the second gear 3552 is moved, the base plate 3560 may be rotated relative to the housing 3201 as the second gear 3552 is moved along the first gear 3551. The roll motor rotation shaft 35141 is eccentric with respect to the rotation shaft of the base plate 3560, and thus, when the second gear 3552 is moved along the first gear 3551, the base plate 3560 may be rotated relative to the housing 3201 rather than changing a position along the second gear 3552.

In some embodiments, as described below, the bearing plate 3540 may also be connected to the second gear 3552 by the roll motor rotation shaft 35141. Accordingly, when the second gear 3552 is rotated, the bearing plate 3540 may be rotated relative to the housing 3201 as the second gear 3552 is moved along the first gear 3551. In this case, as described below, a bearing 3541 for reducing rotational friction of the bearing plate 3540 may be arranged coaxially with the bearing plate 3540 to be in contact with the inner circumferential surface of the housing 3201. Accordingly, the bearing plate 3540 may be easily rotated.

In some embodiments, the gear teeth of the first gear 3551 and the second gear 3552 are illustrated as spur gears, but the disclosure is not limited thereto, and the gear teeth of the first gear 3551 and the second gear 3552 may have various gear shapes, such as helical gears or herringbone gears.

In some embodiments, when the roll driving motor 3514 is arranged outside the motor pack 3510, the first gear 3551 and the second gear 3552 may be formed at different positions.

In an embodiment, the power generation part 3500 may further include a bearing plate 3540 and a first bearing 3541. When the motor pack 3510 performs a roll rotation, the bearing plate 3540 and the first bearing 3541 may reduce rotational friction between the motor pack 3510 and the housing 3201.

The bearing plate 3540 may be arranged in front of the first gear 3551.

At least one through hole may be formed in the bearing plate 3540. The through hole is a portion through which the rotation shafts of the respective motors pass.

For example, the yaw motor rotation shaft 35111 may be formed to extend from the main body of the yaw driving motor 3511 to pass through the base plate 3560 and the bearing plate 3540. In some embodiments, the pitch motor rotation shaft 35121 may be formed to extend from the main body of the pitch driving motor 3512 to pass through the base plate 3560 and the bearing plate 3540. In some embodiments, the firing motor rotation shaft 35131 may be formed to extend from the main body of the firing driving motor 3513 to pass through the base plate 3560 and the bearing plate 3540. In this case, the roll motor rotation shaft 35141 may be formed to extend from the main body of the roll driving motor 3514 to pass through the base plate 3560, but may not extend up to the bearing plate 3540.

Because the yaw motor rotation shaft 35111, the pitch motor rotation shaft 35121, and the firing motor rotation shaft 35131 extend to pass through the bearing plate 3540, the base plate 3560 and the bearing plate 3540 may be rotated together when the motor pack 3510 is rotated.

The first bearing 3541 may be arranged on the outer circumferential surface of the bearing plate 3540. For example, the first bearing 3541 may be arranged to cover the outer circumferential surface of the bearing plate 3540. Accordingly, when the motor pack 3510 performs a roll rotation, the bearing plate 3540 may be rotated together with the motor pack 3510. At this time, the bearing plate 3540 and the first bearing 3541 may reduce rotational friction between the motor pack 3510 and the housing 3201.

In an embodiment, the power generation part 3500 may further include a circuit plate 3570 and a second bearing 3571. When the motor pack 3510 performs a roll rotation, the circuit plate 3570 and the second bearing 3571 may reduce rotational friction between the motor pack 3510 and the housing 3201.

The circuit plate 3570 may be arranged at the rear of the motor pack 3510.

The circuit plate 3570 may be a portion to which a circuit unit 3600 is connected, as described below.

The circuit plate 3570 may be connected to the motor pack 3510. Accordingly, when the motor pack 3510 is rotated, the circuit plate 3570 may be rotated together.

The second bearing 3571 may be arranged on the outer circumferential surface of the circuit plate 3570. Accordingly, when the motor pack 3510 performs a roll rotation, the circuit plate 3570 may be rotated together with the motor pack 3510. At this time, the circuit plate 3570 and the second bearing 3571 may reduce rotational friction between the motor pack 3510 and the housing 3201.

The power generation part 3500 may further include a pulley coupling plate 3530.

The pulley coupling plate 3530 may be a portion to which the power transmission part 3300 is connected.

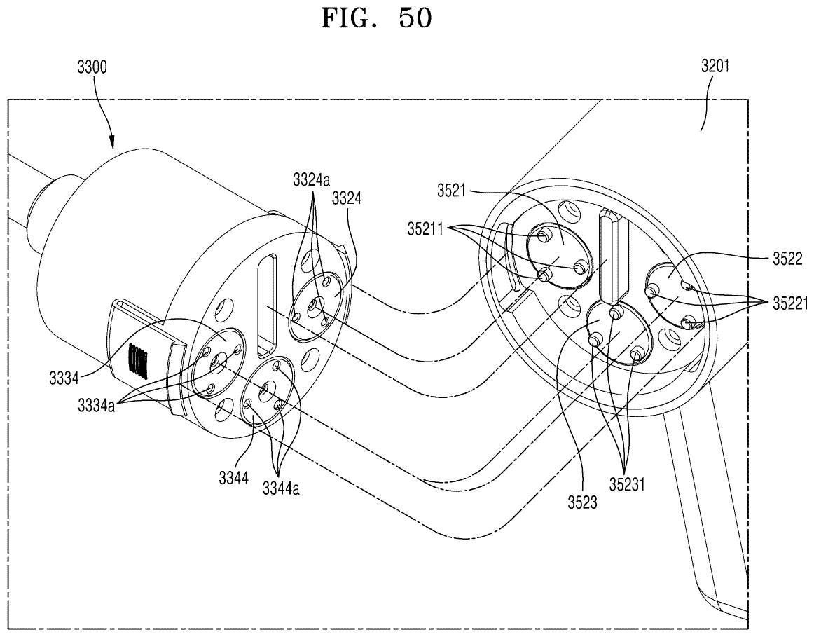

FIG. 50 is a diagram for describing the coupling structure of the surgical instrument according to the second embodiment. Referring to FIG. 50, in an embodiment, the power transmission part 3300 may be detachably fastened to the power generation part 3500. For example, the power transmission part 3300 may be detachably fastened to the pulley coupling plate 3530. Accordingly, after the user uses the components (the power transmission part 3300, the connection part 3400, and the end tool 1100) in a direction from the power transmission part 3300 toward the distal end, the user may discard the components and use the surgical instrument 3000 by coupling a new product again to the manipulation part 3200 which accommodates the power generation part 3500.

In an embodiment, at least one coupling member 3316 may be formed in the pulley frame 3310 of the power transmission part 3300. A hook 33161 may be formed in the coupling member 3316. In some embodiments, the pulley coupling plate 3530 may include an inner space for accommodating at least a portion of the pulley frame 3310 and a wall surface formed along the circumference of the pulley coupling plate 3530 so as to define the inner space. At this time, a hook groove 3532 to which the hook 33161 is caught and fixed may be formed in the wall surface. Accordingly, when the pulley frame 3310 is inserted into the inner space of the pulley coupling plate 3530, the hook 33161 may be inserted into and fixedly fixed to the hook groove 3532.

In an embodiment, the pulley coupling plate 3530 may include a coupling block 3531 formed to protrude, and the pulley frame 3310 may include an insertion groove into which the coupling block 3531 is inserted. When the pulley coupling plate 3530 and the pulley frame 3310 are coupled to each other, the coupling block 3531 is inserted into the insertion groove. Accordingly, the pulley coupling plate 3530 and the pulley frame 3310 may be coupled to each other at a set position. In some embodiments, because the coupling block 3531 is inserted into the insertion groove, the roll rotation force of the pulley coupling plate 3530 may be transmitted to the pulley frame 3310 by the coupling block 3531 and the insertion groove. In other embodiments, the coupling block 3531 may be formed in an elongated bar shape. In this case, the insertion groove may be formed in the corresponding shape.

In an embodiment, although not illustrated, the surgical instrument 3000 according to the disclosure may further include a waterproof structure.

In other embodiments, at least one O-ring may be provided in the housing 3201. For example, the O-ring may be provided between the outer circumferential surface of the pulley coupling plate 3530 and the housing 3201. The O-ring may be arranged in close contact between the outer circumferential surface of the pulley coupling plate 3530 and the housing 3201 to prevent infiltration of water or the like between the power generation part 3500 and the housing 3201.

As another example, the O-ring may be provided between the motor plate 3521 and the pulley coupling plate 3530 and/or between the pulley motor plate 3522 and the pulley coupling plate 3530. The O-ring may be arranged in close contact between the yaw/pitch motor plates 3521 and 3522 and the pulley coupling plate 3530 to prevent infiltration of water or the like between the yaw/pitch motor plates 3521 and 3522 and the pulley coupling plate 3530.

In an embodiment, the pulley coupling plate 3530 and the bearing plate 3540 may be fastened to each other by at least one bolt. In this case, at least one seal washer may be arranged under the bolt in a bolt hole into which the bolt is inserted. The seal washer may prevent infiltration of water or the like through the bolt hole.

At least one through hole may be formed in the pulley coupling plate 3530. For example, two through holes may be formed in the pulley coupling plate 3530.

The yaw motor plate 3521, the pitch motor plate 3522, and the firing motor plate 3523 may be arranged in the through holes formed in the pulley coupling plate 3530.

The yaw motor plate 3521 may be rotated by the driving force generated by the yaw driving motor 3511. The yaw motor plate 3521 may be arranged at an end of the yaw motor rotation shaft 35111. For example, when the yaw motor rotation shaft 35111 is rotated, the yaw motor plate 3521 may be rotated together. From another viewpoint, it may be stated that the yaw motor plate 3521 is a member which transmits the driving force generated by the yaw driving motor 3511 to the power transmission part 3300.

At least one first protrusion 35211 may be formed in the yaw motor plate 3521. The first protrusion 35211 may be a portion which protrudes outward from the yaw motor plate 3521. As described above, the first protrusion 35211 may be inserted into a first insertion hole 3324a formed in a yaw pulley plate 3324.

The pitch motor plate 3522 may be rotated by the driving force generated by the pitch driving motor 3512. The pitch motor plate 3522 may be arranged at an end of the pitch motor rotation shaft 35121. For example, when the pitch motor rotation shaft 35121 is rotated, the pitch motor plate 3522 may be rotated together. From another viewpoint, it may be stated that the pitch motor plate 3522 is a member which transmits the driving force generated by the pitch driving motor 3512 to the power transmission part 3300.

At least one second protrusion 35221 may be formed in the pitch motor plate 3522. The second protrusion 35221 may be a portion which protrudes outward from the pitch motor plate 3522. As described above, the second protrusion 35221 may be inserted into a second insertion hole 3334a formed in the pitch pulley plate 3334.

The firing motor plate 3523 may be rotated by the driving force generated by the firing driving motor 3513. The firing motor plate 3523 may be arranged at an end of the firing motor rotation shaft 35131. For example, when the firing motor rotation shaft 35131 is rotated, the firing motor plate 3523 may be rotated together. From another viewpoint, it may be stated that the firing motor plate 3523 is a member which transmits the driving force generated by the firing driving motor 3513 to the power transmission part 3300.

At least one third protrusion 35231 may be formed in the firing motor plate 3523. The third protrusion 35231 may be a portion which protrudes outward from the firing motor plate 3523. As described below, the third protrusion 35231 may be inserted into a third insertion hole 3344a formed in the firing pulley plate 3344.

The yaw pulley plate 3324, the pitch pulley plate 3334, and the firing pulley plate 3344 may be arranged in the pulley frame 3310.

The yaw pulley plate 3324 may be formed to be rotatable. For example, the yaw pulley plate 3324 may be fastened to the yaw motor plate 3521. When the yaw motor plate 3521 is rotated, the yaw pulley plate 3324 may be rotated together. The yaw pulley plate 3324 may be a portion connected to the yaw driving pulley 3320. When the yaw pulley plate 3324 is rotated, the yaw driving pulley 3320 may be rotated together. From another viewpoint, when power transmitted from the outside rotates the yaw pulley plate 3324, the yaw driving pulley 3320 may be rotated together. From another viewpoint, it may be stated that the yaw pulley plate 3324 is a portion which receives the driving force generated by the yaw driving motor 3511 and transmits the driving force to the yaw driving pulley 3320.

The yaw pulley plate 3324 may include at least one first insertion hole 3324a. The first insertion hole 3324a may be a portion into which the first protrusion 35211 of the yaw motor plate 3521 is inserted. Accordingly, the yaw motor plate 3521 and the yaw pulley plate 3324 may be stably coupled to each other by the coupling of the at least one first protrusion 35211 and the at least one first insertion hole 3324a, and the driving force of the yaw driving motor 3511 may be efficiently transmitted to the yaw driving pulley 3320.

The pitch pulley plate 3334 may be formed to be rotatable. For example, the pitch pulley plate 3334 may be fastened to the pitch motor plate 3522. When the pitch motor plate 3522 is rotated, the pitch pulley plate 3334 may be rotated together. The pitch pulley plate 3334 may be a portion which is connected to the pitch driving pulley 3330. When the pitch pulley plate 3334 is rotated, the pitch driving pulley 3330 may be rotated together. From another viewpoint, when power transmitted from the outside rotates the pitch pulley plate 3334, the pitch driving pulley 3330 may be rotated together. From another viewpoint, it may be stated that the pitch pulley plate 3334 is a portion which receives the driving force generated by the pitch driving motor 3512 and transmits the driving force to the pitch driving pulley 3330.

The pitch pulley plate 3334 may include at least one second insertion hole 3334a. The second insertion hole 3334a may be a portion into which the second protrusion 35221 of the pitch motor plate 3522 is inserted. Accordingly, the pitch motor plate 3522 and the pitch pulley plate 3334 may be stably coupled to each other by the coupling of the at least one second protrusion 35221 and the at least one second insertion hole 3334a, and the driving force of the pitch driving motor 3512 may be efficiently transmitted to the pitch driving pulley 3330.

The firing pulley plate 3344 may be formed to be rotatable. For example, the firing pulley plate 3344 may be fastened to the firing motor plate 3523. When the firing motor plate 3523 is rotated, the firing pulley plate 3344 may be rotated together. The firing pulley plate 3344 may be a portion connected to the firing driving pulley 3340. When the firing pulley plate 3344 is rotated, the firing driving pulley 3340 may be rotated together. From another viewpoint, when power transmitted from the outside rotates the firing pulley plate 3344, the firing driving pulley 3340 may be rotated together. From another viewpoint, it may be stated that the firing pulley plate 3344 is a portion which receives the driving force generated by the firing driving motor 3513 and transmits the driving force to the firing driving pulley 3340.

The firing pulley plate 3344 may include at least one third insertion hole 3344a. The third insertion hole 3344a may be a portion into which the third protrusion 35231 of the firing motor plate 3523 is inserted. Accordingly, the firing motor plate 3523 and the firing pulley plate 3344 may be stably coupled to each other by the coupling of the at least one third protrusion 35231 and the at least one third insertion hole 3334a, and the driving force of the firing driving motor 3513 may be efficiently transmitted to the firing driving pulley 3340.

In an embodiment, the yaw driving motor 3511, the pitch driving motor 3512, the roll driving motor 3514, and the firing driving motor 3513 may be driven independently of each other. Accordingly, the yaw driving motor 3511, the pitch driving motor 3512, the roll driving motor 3514, and the firing driving motor 3513 may independently perform the yaw rotation of the end tool 1100, the pitch rotation of the end tool 1100, the roll rotation of the motor pack 3510, and the linear motion of the operation member 1140.

Referring again to FIG. 48, as the roll driving motor 3514 is driven, the pulley coupling plate 3530 may be rotated in an A direction. At this time, because the yaw driving motor 3511 may be driven independently, the yaw driving motor 3511 may be driven independently regardless of the driving of the roll driving motor 3514 to rotate the yaw motor plate 3521 in a B direction. In some embodiments, because the pitch driving motor 3512 may be driven independently, the pitch driving motor 3512 may be driven independently of the driving of the roll driving motor 3514 and the yaw driving motor 3511 to rotate the pitch motor plate 3522 in a C direction. In some embodiments, because the firing driving motor 3513 may be driven independently, the firing driving motor 3513 may be driven independently of the driving of the roll driving motor 3514, the yaw driving motor 3511, and the pitch driving motor 3512 to rotate the firing motor plate 3523 in a D direction. From another viewpoint, the end tool 1100 may perform only one of the pitch rotation, the yaw rotation, the roll rotation, and the linear motion of the operation member 1140, or may perform the plurality of motions simultaneously.

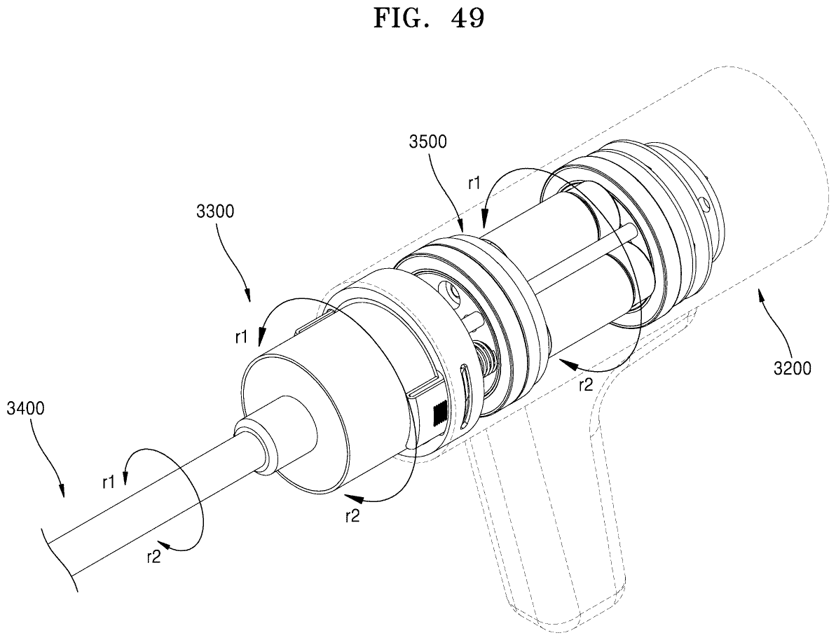

FIG. 49 is a diagram for describing the roll motion of the surgical instrument according to the second embodiment.

Referring to FIG. 49, the surgical instrument according to an embodiment may be formed so that the motor pack 3510 may perform a roll rotation.

The power generation part including the motor pack 3510 may include the pulley frame 3310 coupled to the power transmission part in the forefront. The pulley frame 3310 may be coupled to the power transmission part. Accordingly, the power transmission part 3300 may perform a roll rotation together with the pulley coupling plate 3530 in a state of being fastened to the pulley coupling plate 3530.

In some embodiments, the connection part 3400 may be connected to the power transmission part 3300. Accordingly, the connection part 3400 may perform a roll rotation together due to the roll rotation of the power transmission part 3300.

In some embodiments, the end tool 1100 arranged on a side of the connection part 3400 may be connected to the connection part 3400. Accordingly, the end tool 1100 may perform a roll rotation together due to the rotation of the connection part 3400.

As a result, according to the disclosure, when the user manipulates the manipulation part 3200 to drive the roll driving motor 3514, the configuration excluding the manipulation part 3200 may perform a roll rotation with respect to the longitudinal direction in which the connection part 3400 extends.

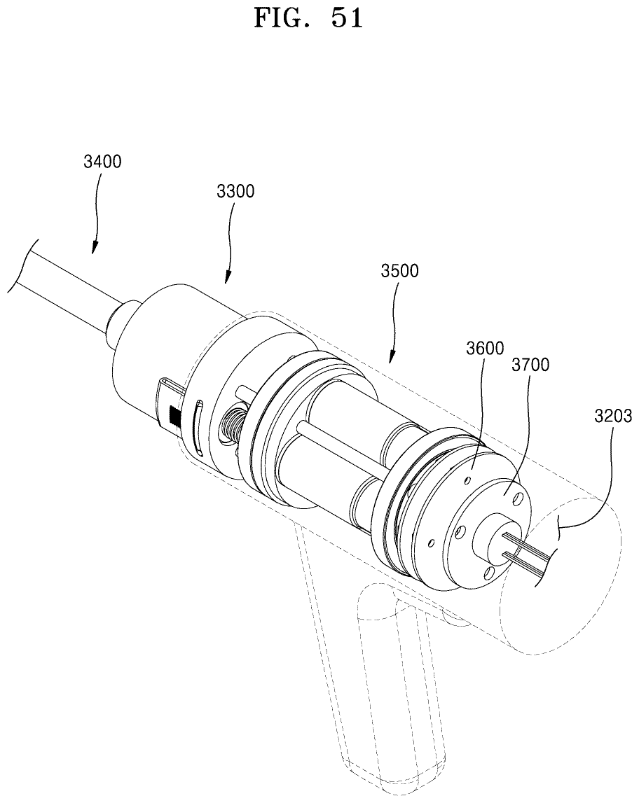

FIG. 51 is a diagram illustrating the internal structure of the surgical instrument according to the second embodiment.

Referring to FIG. 51, a manipulation part inner space 3203 may be provided in the manipulation part 3200.

A circuit unit 3600 may be arranged in the manipulation part inner space 3203.

The circuit unit 3600 is a configuration including an electronic circuit for controlling the driving of the motor pack 3510. As an example, the circuit unit 3600 may include a motor driver, a motor controller, and/or a microcontroller unit, but the disclosure is not limited thereto, and any circuit unit may be used as long as the circuit unit drives the motor pack 3510.

The circuit unit 3600 may be arranged on a side of the motor pack 3510. For example, the circuit unit 3600 may be arranged at the rear of the motor pack 3510 (i.e., opposite the connection part 3400) with reference to FIG. 51. For example, the circuit unit 3600 may be connected to the circuit plate 3570 of the power generation part 3500. Accordingly, when the motor pack 3510 performs a roll rotation, the circuit unit 3600 may be rotated together with the motor pack 3510.

Although not illustrated, the circuit unit 3600 and the motor pack 3510 may be connected to each other through a plurality of electric wires so as to drive the motor pack 3510. Accordingly, as the motor pack 3510 performs a roll rotation, the circuit unit 3600 may also be rotated, and thus, the problem that the plurality of electric wires connecting the motor pack 3510 to the circuit unit 3600 are twisted may be prevented.

In an embodiment, a slip ring 3700 may be arranged on a side of the circuit unit 3600. The slip ring 3700 is a configuration which connects various electrical/electronic elements to the motor pack 3510 or the circuit unit 3600, which controls the driving of the motor pack 3510, or connects communication. For example, the slip ring 3700 may electrically connect the motor pack 3510 or the circuit unit 3600, which controls the driving of the motor pack 3510, to a power source, a switch, a button, an organic light-emitting diode (OLED) screen, or other circuit units. The power source, the switch, the button, the OLED screen, or other circuit units may be arranged inside or outside the surgical instrument 3000 according to the disclosure. In some embodiments, the slip ring 3700 may connect communication between various elements for the operation of the surgical instrument 3000. For example, the slip ring 3700 may connect communication between at least some of the manipulation part 3200, the power transmission part 3300, the power generation part 3500, and the circuit unit 3600. At this time, the type of communication is not limited, and any type of communication may be adopted as long as various elements of the surgical instrument 3000 are communicatively connected.

As described above, the motor pack 3510 and the circuit unit 3600 may be formed to enable a roll rotation. In some embodiments, the motor pack 3510 and/or the circuit unit 3600 may be electrically connected to various electrical/electronic elements. In this case, when the motor pack 3510 and/or the circuit unit 3600 are connected to the electrical/electronic elements through electric wires or the like, the problem that the electric wires are twisted by the rotation of the motor pack 3510 and the circuit unit 3600 may occur.

Accordingly, because the slip ring 3700 is arranged on a side of the circuit unit 3600, the electric wires connecting the motor pack 3510 and/or the circuit unit 3600 to various electrical/electronic elements may not be twisted even when the circuit unit 3600 is rotated. For example, because the electric wires are not twisted, the motor pack 3510 and the circuit unit 3600 may stably receive power from an external power source.

In an embodiment, although not illustrated, the surgical instrument may further include at least one sub-circuit unit. The sub-circuit unit may be arranged in the manipulation part 3200. In other embodiments, the sub-circuit unit may be arranged in the handle (3202 of FIG. 43) of the manipulation part 3200. In this case, the sub-circuit unit may not be rotated even when the motor pack 3510 is rotated.

The sub-circuit unit may preprocess various signals for controlling the motor pack 3510. The sub-circuit unit may transmit the preprocessed signals to the circuit unit 3600. To this end, the circuit unit 3600 and the sub-circuit unit may be connected to each other by serial communication or the like, but the disclosure is not limited thereto, and the circuit unit 3600 and the sub-circuit unit may be connected to each other in various manners. Accordingly, the number of electric wires which need to be connected to the circuit unit 3600 through the slip ring 3700 may be reduced.

For example, when it is assumed that there are four buttons for manipulation on the manipulation part 3200 and two electric wires (for example, a ground electric wire and a communication electric wire) are required for each button so as to transmit and receive signals, at least five electric wires have to be connected to the circuit unit 3600. For example, even when the ground electric wire is commonly used, at least one ground electric wire and four communication electric wires are required are required for the manipulation of the manipulation part 3200. In this case, when the sub-circuit unit is included and signals of at least five electric wires are preprocessed once in the sub-circuit unit as in the present embodiment, the circuit unit 3600 and the four buttons of the manipulation part 3200 may perform communication with only two electric wires through the communication connection function of the slip ring 3700. Therefore, this configuration may simplify the layout of electric wires and may also minimize the size of the slip ring 3700. However, this example is only one of the plurality of functions of the sub-circuit unit, and the sub-circuit unit may preprocess signals of the plurality of electric wires, and thus, the technical contents of the disclosure are not limited to the above description.

In an embodiment, although not illustrated, the surgical instrument may further include a configuration which sets the zero point of roll rotation of the motor pack 3510 or the like. For example, the surgical instrument may further include at least one encoder which measures the roll rotation angle of the motor pack 3510 or the like. For example, the surgical instrument may further include a touch sensor, a hall effect sensor, a photo sensor, or the like so as to measure the roll rotation angle of the motor pack 3510 or the like. However, the disclosure is not limited thereto, and any device may be included in the surgical instrument of the disclosure as long as the device measures the roll rotation angle of the motor pack 3510 or the like.

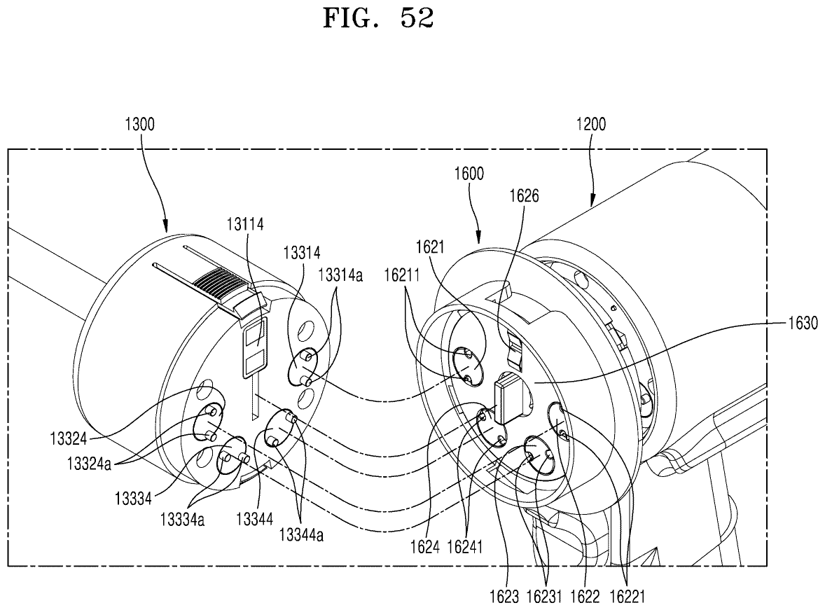

FIG. 52 is a diagram for describing the coupling structure of the surgical instrument according to the first embodiment.

Referring to FIG. 52, the surgical instrument according to the first embodiment differs from the surgical instrument according to the second embodiment, in that the number of driving motors in the motor pack is different, and accordingly, the number of driving pulleys in the power transmission part is different.

Because the common contents related to the manipulation part 1200, the power generation part 1500, and the power transmission part 1300 are the same as those described above in the surgical instrument according to the second embodiment, only the distinctively different parts are described.

The surgical instrument according to the first embodiment may include a drape device arranged between the power transmission part and the manipulation part.

The drape device may be a device which is fastened to the manipulation part and wraps a non-sterile manipulation part with a sterile drape so as to enable the use of the surgical instrument in a sterile state. For example, the user in the sterile region may use the surgical instrument in the sterile region by holding the manipulation part wrapped in the sterile drape.

The power transmission part according to the first embodiment may be directly coupled to the manipulation part, or may also be connected to the manipulation part through the drape device.

For example, in the surgical instrument according to the second embodiment, as described above with respect to the coupling structure of the power transmission part and the power generation part, the coupling structure of the drape device and the power generation part according to the first embodiment may be substantially the same as described above. For example, the motor plate and the pulley plate may be engaged with and coupled to each other so as to be rotated together.

In some embodiments, the coupling structure of the drape device and the power transmission part according to the first embodiment may be substantially the same as described above.

For example, the drape device may have at least one through hole formed in the pulley coupling plate. For example, four through holes may be formed in the pulley coupling plate. Pulley plates may be respectively arranged in the four through holes.

For example, a first firing pulley plate 1621, a second firing pulley plate 1622, a yaw pulley plate 1623, and a pitch pulley plate 1624 may be arranged in the through holes formed in the pulley coupling plate 1630.

The yaw pulley plate 1623 may be rotated by the driving force generated by the yaw driving motor (not shown). The yaw pulley plate 1623 may be arranged at an end of the yaw motor rotation shaft (not shown). For example, when the yaw motor rotation shaft (not shown) is rotated, the yaw pulley plate 1623 may be rotated together. From another viewpoint, it may be stated that the yaw pulley plate 1623 is a member which transmits the driving force generated by the yaw driving motor (not shown) to the drape device 1600.

At least one first insertion hole 16231 may be formed in the yaw pulley plate 1623. The first insertion hole 16231 may be a portion which is recessed inward from the yaw pulley plate 1623. As described below, a first protrusion 13334a formed in the yaw driving pulley plate 13334 of the power transmission part 1300 may be inserted into the first insertion hole 16231.

Because the pitch pulley plate 1624, the first firing pulley plate 1621, and the second firing pulley plate 1622 are substantially identical to the yaw pulley plate 1623 in the corresponding ranges, a detailed description thereof is omitted.

In some embodiments, as described above, the power transmission part 1300 according to the first embodiment may include a first firing driving pulley plate 13314, a second firing driving pulley plate 13324, a yaw driving pulley plate 13334, and a pitch driving pulley plate 13344, which are arranged in the pulley frame 1310.

The yaw driving pulley plate 13334 may be formed to be rotatable. For example, the yaw driving pulley plate 13334 may be fastened to the yaw pulley plate 1623. When the yaw pulley plate 1623 is rotated, the yaw driving pulley plate 13334 may be rotated together. The yaw driving pulley plate 13334 may be a portion connected to the yaw driving pulley 1333. When the yaw driving pulley plate 13334 is rotated, the yaw driving pulley 1333 may be rotated together. From another viewpoint, when power transmitted from the outside rotates the yaw driving pulley plate 13334, the yaw driving pulley 1333 may be rotated together. From another viewpoint, it may be stated that the yaw driving pulley plate 13334 is a portion which receives the driving force generated by the yaw driving motor (not shown) and transmits the driving force to the yaw driving pulley 1333.

The yaw driving pulley plate 13334 may include at least one first protrusion 13334a. The first protrusion 13334a may be a portion inserted into the first insertion hole 16231 of the yaw pulley plate 1623. Accordingly, the yaw pulley plate 1623 and the yaw driving pulley plate 13334 may be stably coupled to each other by the coupling of the at least one first protrusion 13334a and the at least one first insertion hole 16231, and the driving force of the yaw driving motor (not shown) may be efficiently transmitted to the yaw driving pulley 1333.

The pitch driving pulley plate 13344 may include at least one second protrusion 13344a. The second protrusion 13344a may be a portion inserted into the second insertion hole 16241 of the pitch pulley plate 1624. Accordingly, the pitch pulley plate 1624 and the pitch driving pulley plate 13344 may be stably coupled to each other by the coupling of the at least one second protrusion 13344a and the at least one second insertion hole 16241, and the driving force of the pitch driving motor (not shown) may be efficiently transmitted to the pitch driving pulley 1334.

The first firing driving pulley plate 13314 may include at least one third protrusion 13314a. The third protrusion 13314a may be a portion inserted into the third insertion hole 16211 of the first firing pulley plate 1621. Accordingly, the first firing pulley plate 1621 and the first firing driving pulley plate 13314 may be stably coupled to each other by the coupling of the at least one third protrusion 13314a and the at least one third insertion hole 16211, and the driving force of the first firing driving motor (not shown) may be efficiently transmitted to the first firing driving pulley 1331.

Because the coupling relationship between the second firing driving pulley plate 13324 and the second firing pulley plate 1622 is substantially the same as described above, a detailed description thereof is omitted.

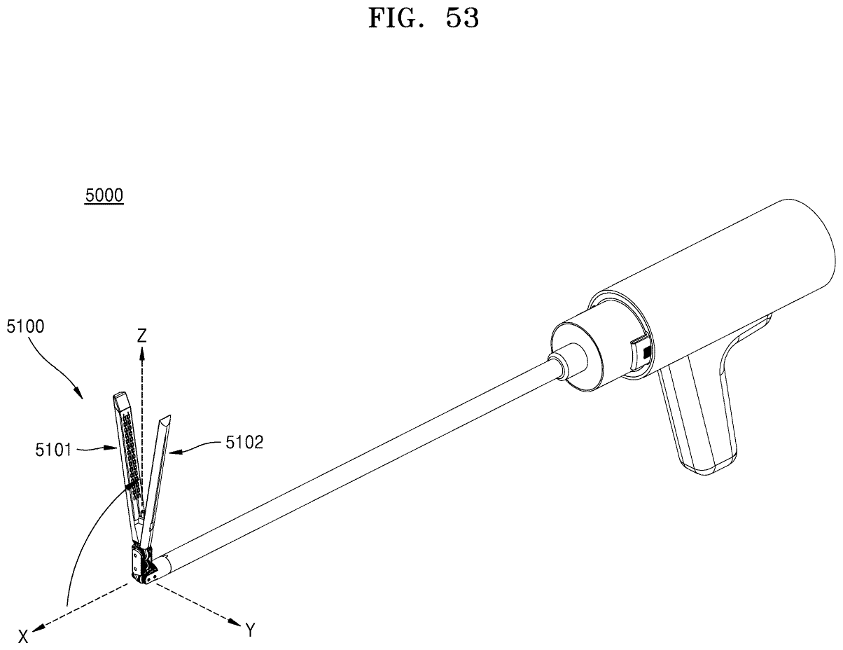

FIGS. 53 to 57 are diagrams illustrating a pitch rotation motion of a surgical instrument according to an embodiment.

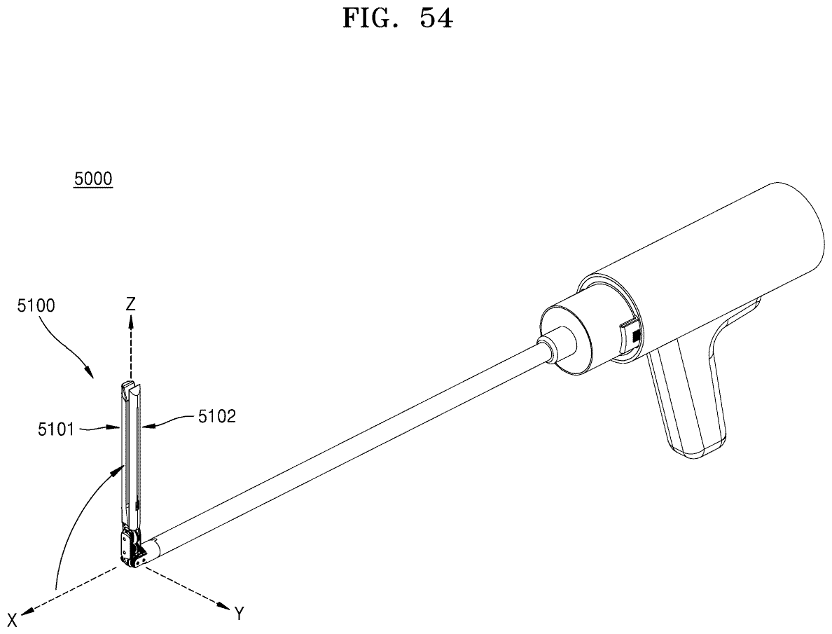

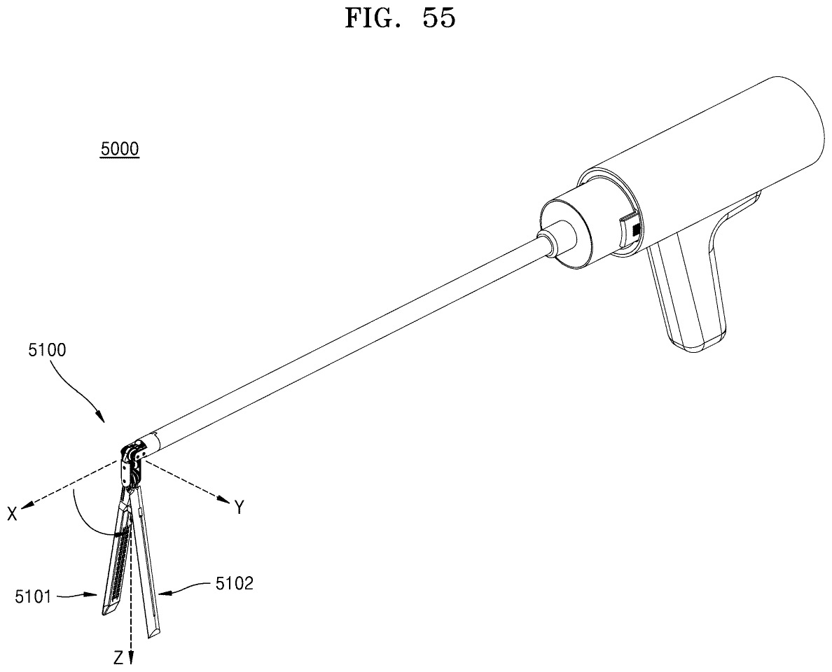

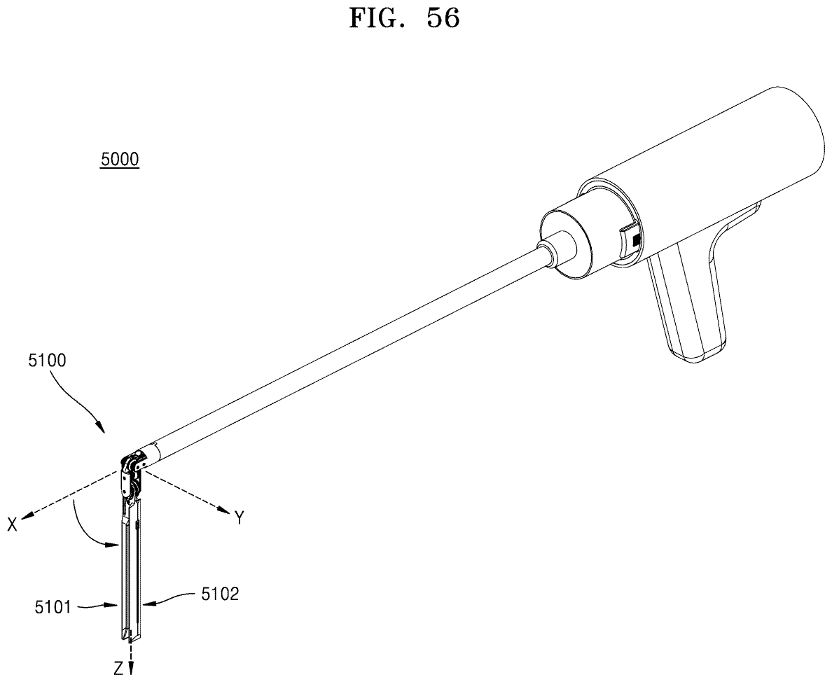

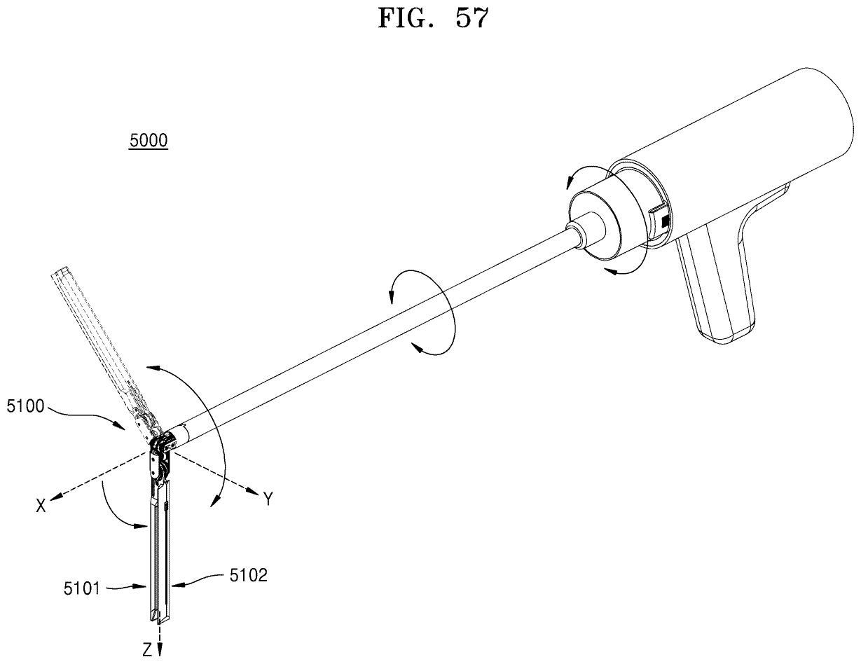

FIG. 53 is a diagram illustrating a state where jaws are pitch-rotated by −90°, and FIG. 54 is a diagram illustrating a process in which an actuation motion is performed in a state where the jaws are pitch-rotated by −90°. FIG. 55 is a diagram illustrating a state where the jaws are pitch-rotated by +90°, FIG. 56 is a diagram illustrating a process in which an actuation motion is performed in a state where the jaws are pitch-rotated by +90°, and FIG. 57 is a diagram illustrating a state where a roll motion is performed in a state where the jaws are pitch-rotated.

Referring to FIGS. 53 to 57, a surgical instrument 5000 according to the disclosure may include an end tool 5100 including a first jaw 5101 and a second jaw 5102. The end tool 5100 of the surgical instrument 5000 may be the end tool 1100 described with reference to FIGS. 1 to 8. For example, the end tool 5100 may be the end tool 1100 in which at least some configurations are changed or omitted.

For example, the end tool 5100 of the surgical instrument 5000 is not limited to a stapler, and configurations such as a surgical grasper, a vessel sealer, or a single-arm cauterizer may also be used as the end tool.

The end tool 5100 of the surgical instrument 5000 may be pitch-rotated in the positive (+) direction with respect to the pitch rotation shaft (Y-axis). At this time, the first jaw 5101 and the second jaw 5102 of the end tool 5100 may perform an actuation motion in a state where the end tool 5100 is pitch-rotated in the positive (+) direction with respect to the pitch rotation shaft (Y-axis).

In some embodiments, the end tool 5100 of the surgical instrument 5000 may pitch-rotate in the negative (−) direction with respect to the pitch rotation shaft (Y-axis). At this time, the first jaw 5101 and the second jaw of the end tool 5100 may perform an actuation motion in a state where the end tool 5100 is pitch-rotated in the negative (−) direction with respect to the pitch rotation shaft (Y-axis).

The rotation angle of the end tool 5100 may be variously set according to the ratio of the pulley.

In some embodiments, the end tool 5100 of the surgical instrument 5000 may not be rotated with respect to the pitch rotation shaft (Y-axis), may be pitch-rotate in the positive (+) direction, or may be roll-rotated with respect to the roll rotation shaft (X-axis) in a state of being pitch-rotated in the negative (−) direction. At this time, the end tool 5100 may perform a roll rotation in a state where the first jaw 5101 and the second jaw 5102 are separated from each other, or may perform a roll rotation in a state where the first jaw 5101 and the second jaw 5102 perform an actuation motion.

The end tool 5100 may be mechanically connected to the motor pack of the power generation part and may be rotated together with the motor pack. When the motor pack of the power generation part is rotated, the power transmission part connected to the power generation part, the connection part connected to the power transmission part, and the end tool 5100 formed on a side of the connection part may be simultaneously rotated.

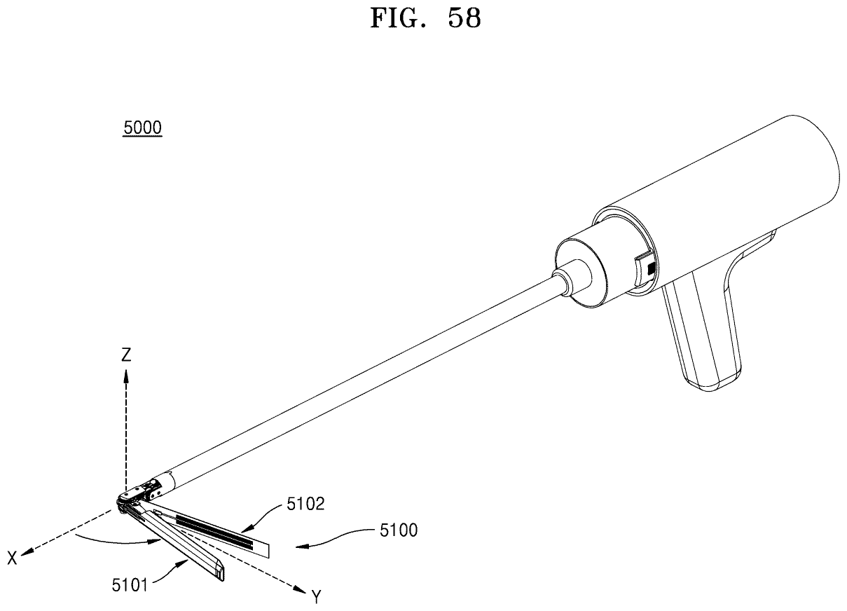

FIGS. 58 to 62 are diagrams illustrating a yaw rotation motion of a surgical instrument according to an embodiment.

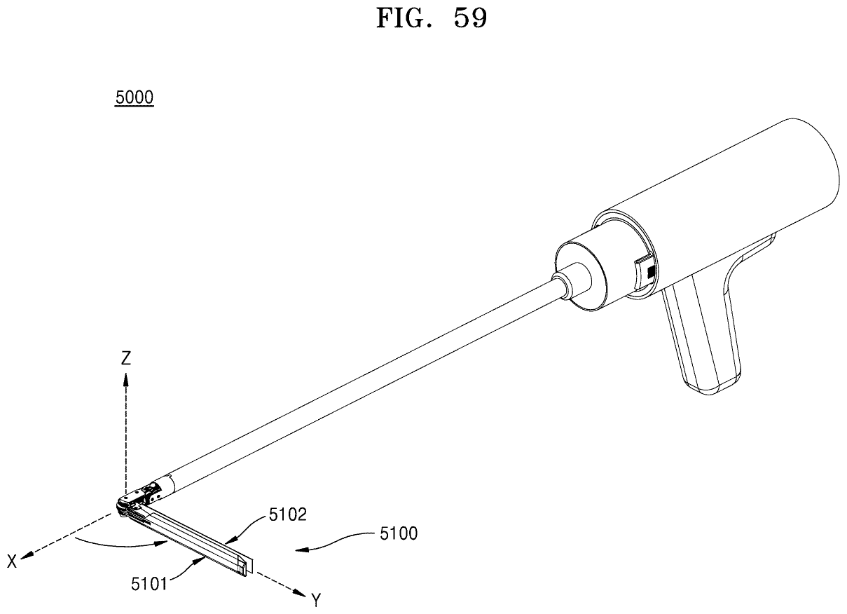

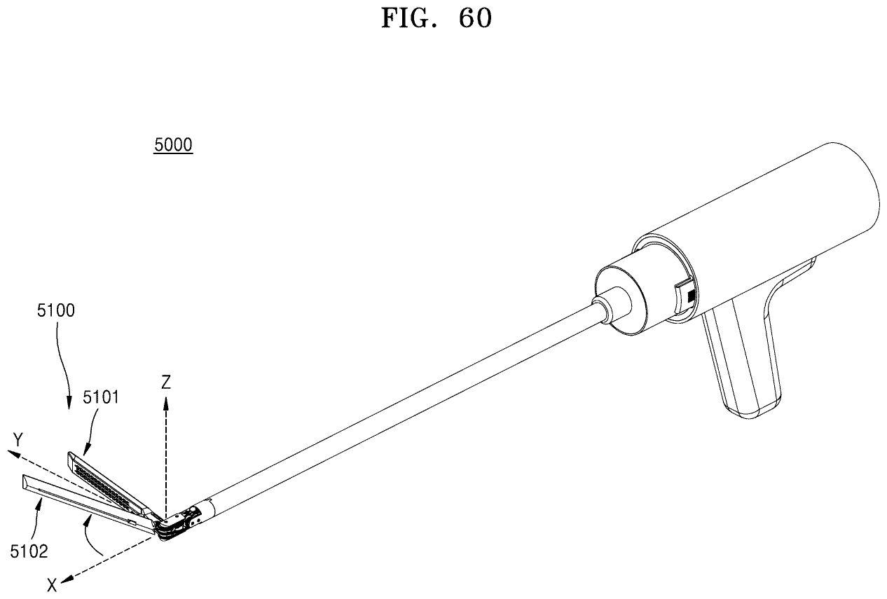

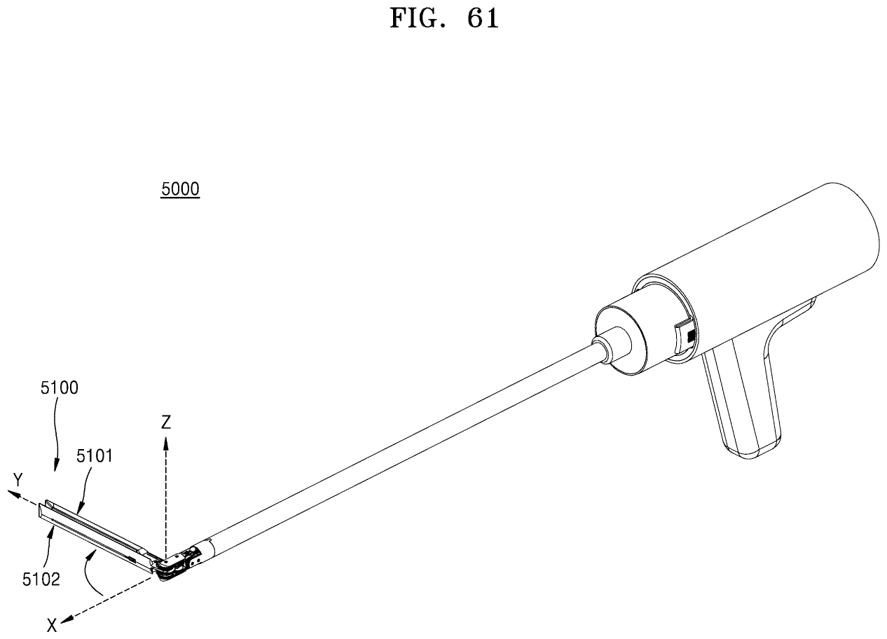

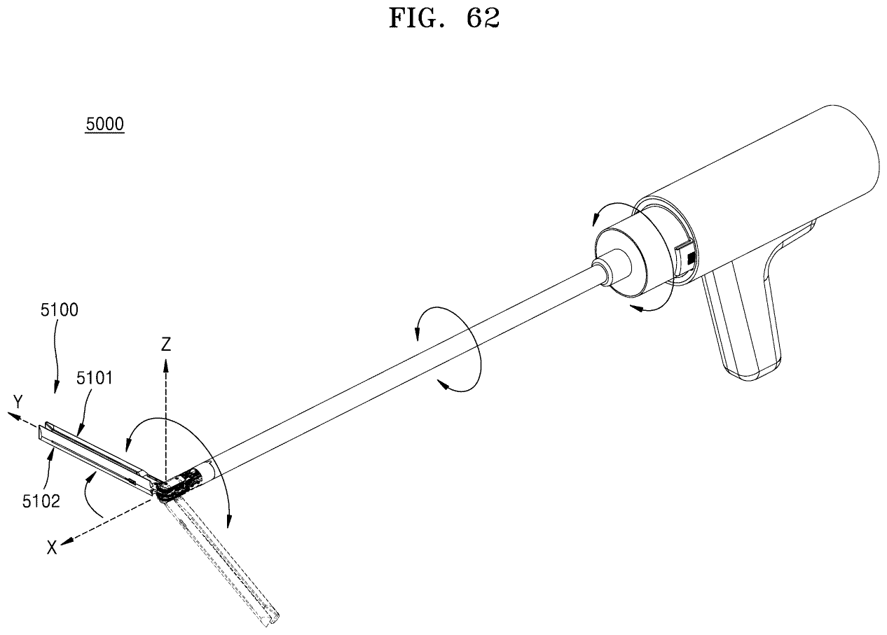

FIG. 58 is a diagram illustrating a state where the jaws are yaw-rotated by −90°, and FIG. 59 is a diagram illustrating a process in which an actuation motion is performed in a state where the jaws are yaw-rotated by −90°. FIG. 60 is a diagram illustrating a state where the jaws are yaw-rotated by +90°, FIG. 61 is a diagram illustrating a process in which an actuation motion is performed in a state where the jaws are yaw-rotated by +90°, and FIG. 62 is a diagram illustrating a state where a roll motion is performed in a state where the jaws are yaw-rotated.

Referring to FIGS. 58 to 62, a surgical instrument 5000 according to the disclosure may include an end tool 5100 including a first jaw 5101 and a second jaw 5102. The end tool 5100 of the surgical instrument 5000 may be the end tool 1100 described with reference to FIGS. 1 to 8. For example, the end tool 5100 may be the end tool 1100 in which at least some configurations are changed or omitted.

For example, the end tool 5100 of the surgical instrument 5000 is not limited to a stapler, and configurations such as a surgical grasper, a vessel sealer, or a single-arm cauterizer may also be used as the end tool.

The end tool 5100 of the surgical instrument 5000 may be yaw-rotated in the positive (+) direction with respect to the yaw rotation shaft (Z-axis). At this time, the first jaw 5101 and the second jaw 5102 of the end tool 5100 may perform an actuation motion in a state where the end tool 5100 is yaw-rotated in the positive (+) direction with respect to the yaw rotation axis (Z-axis).

In some embodiments, the end tool 5100 of the surgical instrument 5000 may yaw-rotate in the negative (−) direction with respect to the yaw rotation axis (Z-axis). At this time, the first jaw 5101 and the second jaw 5102 of the end tool 5100 may perform an actuation motion in a state where the end tool 5100 is yaw-rotated in the negative (−) direction with respect to the yaw rotation shaft (Z-axis).

The rotation angle of the end tool 5100 may be variously set according to the ratio of the pulley.

In some embodiments, the end tool 5100 of the surgical instrument 5000 may not be rotated with respect to the yaw rotation shaft (Z-axis), may be yaw-rotate in the positive (+) direction, or may be roll-rotated with respect to the roll rotation shaft (X-axis) in a state of being yaw-rotated in the negative (−) direction. At this time, the end tool 5100 may perform a roll rotation in a state where the first jaw 5101 and the second jaw 5102 are separated from each other, or may perform a roll rotation in a state where the first jaw 5101 and the second jaw 5102 perform an actuation motion.

The end tool 5100 may be mechanically connected to the motor pack of the power generation part and may be rotated together with the motor pack. When the motor pack of the power generation part is rotated, the power transmission part connected to the power generation part, the connection part connected to the power transmission part, and the end tool 5100 formed on a side of the connection part may be simultaneously rotated.

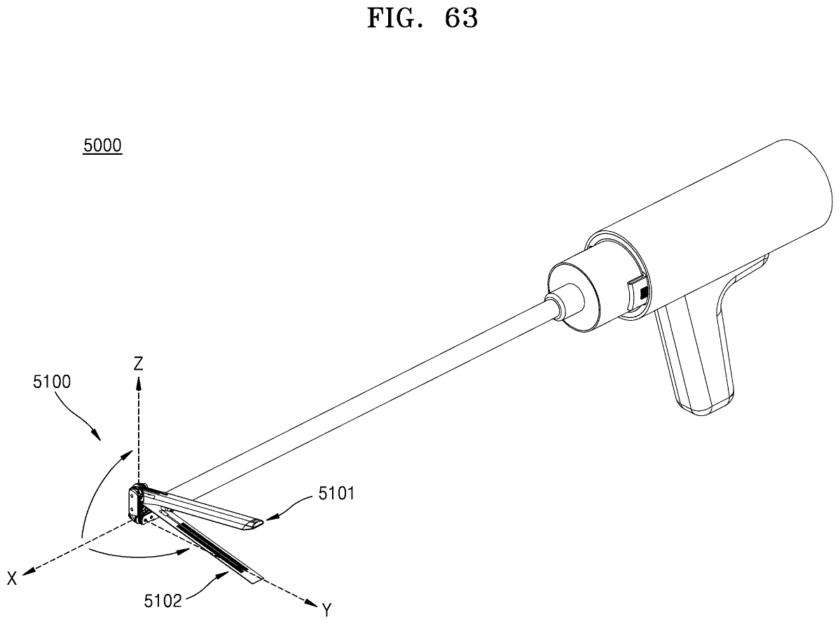

FIGS. 63 to 67 are diagrams illustrating a state where a surgical instrument according to an embodiment performs a pitch rotation motion and a yaw rotation motion.

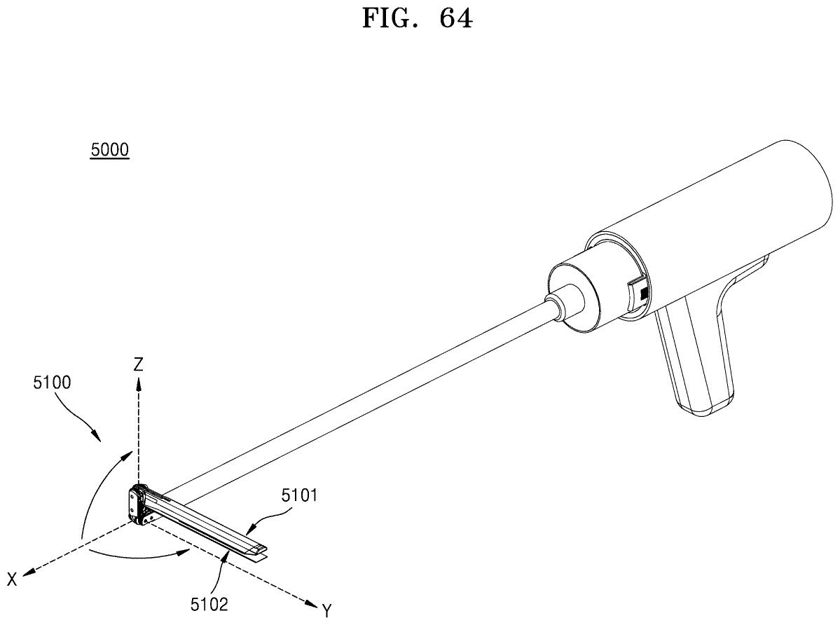

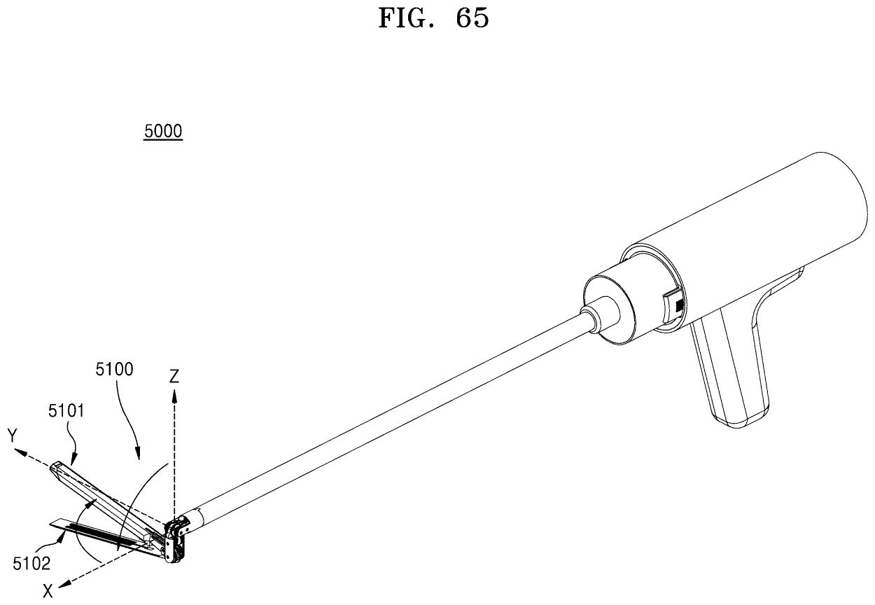

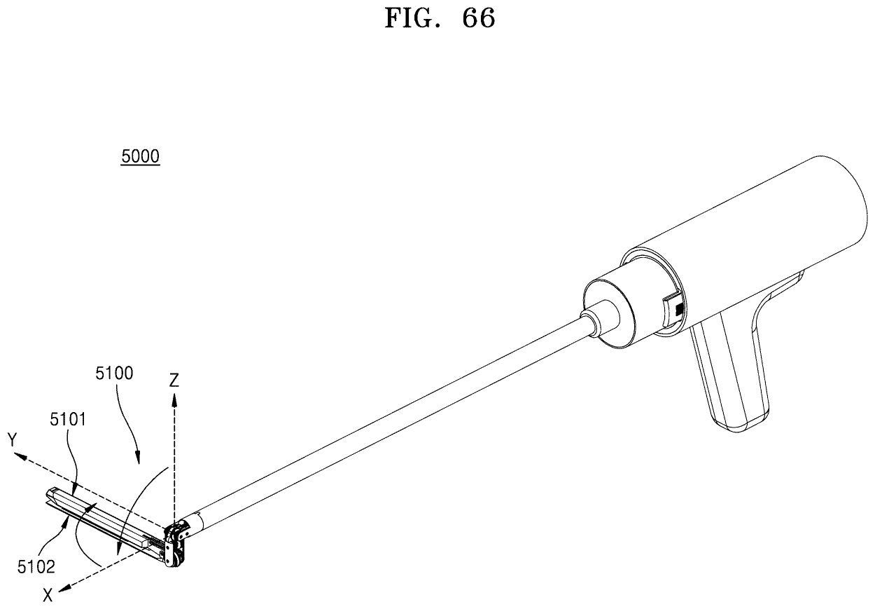

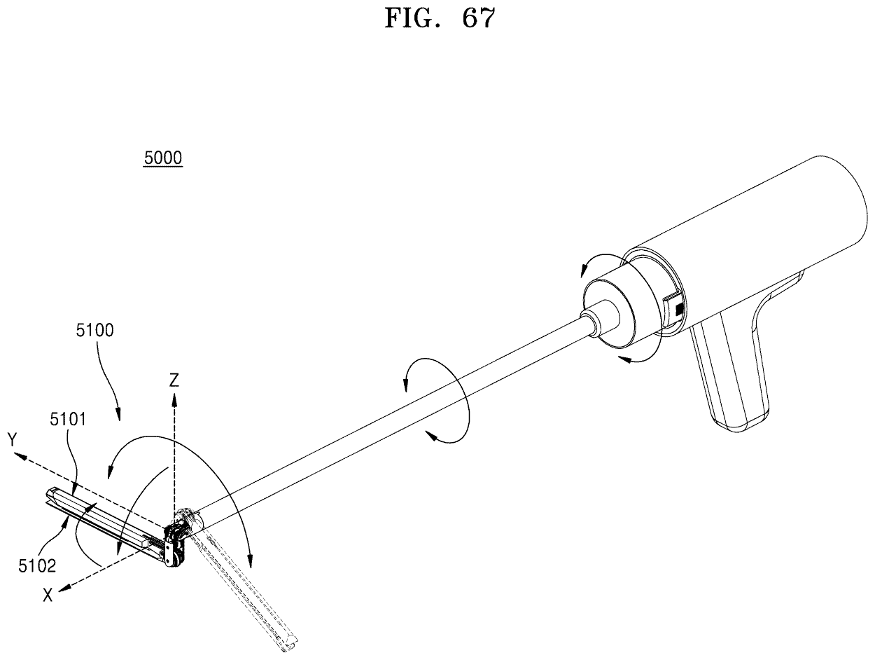

FIG. 63 is a diagram illustrating a state where the jaws are pitch-rotated by −90° and yaw-rotated by +90°, and FIG. 64 is a diagram illustrating a process in which an actuation motion is performed in a state where the jaws are pitch-rotated by −90° and yaw-rotated by +90°. FIG. 65 is a diagram illustrating a state where the jaws are pitch-rotated by +90° and yaw-rotated by −90°, FIG. 66 is a diagram illustrating a process in which an actuation motion is performed in a state where the jaws are pitch-rotated by +90° and yaw-rotated by −90°, and FIG. 67 is a diagram illustrating a state where a roll motion is performed in a state where the jaws are pitch-rotated and yaw-rotated.

Referring to FIGS. 63 to 67, a surgical instrument 5000 according to the disclosure may include an end tool 5100 including a first jaw 5101 and a second jaw 5102. The end tool 5100 of the surgical instrument 5000 may be the end tool 1100 described with reference to FIGS. 1 to 8. For example, the end tool 5100 may be the end tool 1100 in which at least some configurations are changed or omitted.

For example, the end tool 5100 of the surgical instrument 5000 is not limited to a stapler, and configurations such as a surgical grasper, a vessel sealer, or a single-arm cauterizer may also be used as the end tool.

The end tool 5100 of the surgical instrument 5000 may be yaw-rotated with respect to the yaw rotation shaft (Z-axis) and pitch-rotated with respect to the pitch rotation shaft (Y-axis). From another viewpoint, the end tool 5100 of the surgical instrument 5000 may perform a yaw rotation and a pitch rotation simultaneously. At this time, the first jaw 5101 and the second jaw 5102 of the end tool 5100 may perform an actuation motion in a state where the end tool 5100 is yaw-rotated and pitch-rotated.

The rotation angle of the end tool 5100 may be variously set according to the ratio of the pulley.

In some embodiments, the end tool 5100 of the surgical instrument 5000 may be roll-rotated with respect to the roll rotation shaft (X-axis) in a state of being pitch-rotated and yaw-rotated. At this time, the end tool 5100 may perform a roll rotation in a state where the first jaw 5101 and the second jaw 5102 are separated from each other, or may perform a roll rotation in a state where the first jaw 5101 and the second jaw 5102 perform an actuation motion.

The end tool 5100 may be rotated along with the rotation of the motor pack of the power generation part. When the motor pack of the power generation part is rotated, the power transmission part connected to the power generation part, the connection part connected to the power transmission part, and the end tool 5100 formed on a side of the connection part may be simultaneously rotated.

Because the surgical instrument 1000 according to the disclosure is configured so that the motor packs 1510 and 3510, the power transmission parts 1300, and the end tools 1100 and 1100 may be roll-rotated simultaneously, the problem that wires or electric wires are twisted in the surgical instruments 1000 and 3000 may not occur. This has technical significance in that unlimited roll rotation of the surgical instruments 1000 and 3000 is enabled.

For example, in conventional surgical instruments, the pulleys inside the power transmission part are not roll-rotated and only the connection part and the end tool are roll-rotated, which causes the problem that the wires connecting the end tool to the power transmission part are twisted in the connection part. In this case, when the end tool and the connection part continue to perform a roll rotation, the wires may be eventually broken or damaged.

In contrast, in the surgical instruments 1000 and 3000 according to the disclosure, the pulleys 1320, 1330, 3320, 3330, and 3340 inside the end tools 1100 and 1100, the connection parts 1400 and 3400, and the power transmission parts 1300 and 3300 may be roll-rotated together. From another viewpoint, it may be stated that the start points and the end points of the wires 1361, 1362, 1363, 1364, 3361, 3362, 3363, 3364, 3365, and 3366 connecting the pulleys 1320, 1330, 3320, 3330, and 3340 of the end tools 1100 and 1100 and the power transmission parts 3300 are roll-rotated together. Accordingly, there is an effect in that the wires 1361, 1362, 1363, 1364, 3361, 3362, 3363, 3364, 3365, and 3366 are not twisted in the connection parts 1400 and 3400.

According to the disclosure, a surgical instrument capable of an axial rotation (roll) without any limitation of rotation angle may be provided. According to the disclosure, a compact product may be provided by optimizing an arrangement structure of driving pulleys.

The above-described embodiments or modifications may be implemented independently of each other or may be implemented together.

The disclosure has been described with reference to some embodiments. Those of ordinary skill in the art will understand that the disclosure may be implemented in modified forms without departing from the essential features of the disclosure. Therefore, the disclosed embodiments should be considered in an illustrative sense rather than a restrictive sense. The scope of the disclosure is indicated in the claims rather than the foregoing description, and all differences within the scope equivalent thereto should be construed as falling within the disclosure.

What is claimed is:

1. A reload assembly of a surgical instrument, the reload assembly comprising:
an end tool comprising one or more rotatable jaws and configured to be rotated in at least one direction;
a shaft having an end to which the end tool is connected; and
a power transmission part connected to the shaft and configured to transmit power generated by a power generation part to the end tool,
wherein the power transmission part comprises:
a pulley frame connected to the power generation part;
a driving pulley unit accommodated in the pulley frame and configured to receive the power generated by the power generation part and perform an axial rotation; and
a wire unit having one end portion connected to the driving pulley unit and another end portion connected to the end tool and configured to transmit the power generated by the power generation part to the end tool,
wherein a rotation shaft of the driving pulley unit is arranged in a direction extending toward the end tool.

2. The reload assembly of claim 1, wherein
the driving pulley unit comprises a first driving pulley,
the wire unit comprises a first wire wound around the first driving pulley in a first direction and a second wire wound around the first driving pulley in a second direction opposite to the first direction, and
by unidirectional rotation of the first driving pulley, one of the first wire and the second wire is wound around the first driving pulley and another of the first wire and the second wire is unwound from the first driving pulley.

3. The reload assembly of claim 2, wherein
a groove having a shape of a thread root is formed on an outer circumferential surface of the first driving pulley, and
the first wire and the second wire are wound around the groove.

4. The reload assembly of claim 3, wherein
a portion of the first wire fixed to the first driving pulley is constant, and
a portion of the second wire fixed to the first driving pulley is variable.

5. The reload assembly of claim 3, wherein
the groove is formed continuously to allow the first wire and the second wire to be wound around the groove, and
when one of the first wire and the second wire is unwound from a wound position as the first driving pulley is rotated, another of the first wire and the second wire is wound at the wound position.

6. The reload assembly of claim 1, wherein the driving pulley unit comprises a pair of driving pulleys arranged symmetrically with respect to a central axis of the shaft.

7. The reload assembly of claim 1, wherein the driving pulley unit comprises:
at least one firing driving pulley around which a forward wire and a backward wire are wound;
a yaw driving pulley around which a yaw wire is wound; and
a pitch driving pulley around which a pitch wire is wound.

8. The reload assembly of claim 1, wherein the power transmission part further comprises an auxiliary pulley unit contacting at least a portion of the wire unit and configured to change a progressing path of the wire unit extending from the driving pulley unit and guide the wire unit into the shaft.

9. The reload assembly of claim 8,
wherein the driving pulley unit comprises a first driving pulley and a second driving pulley arranged symmetrically with respect to a central axis of the shaft, and
wherein the auxiliary pulley unit comprises a center auxiliary pulley arranged between the first driving pulley and the second driving pulley.

10. The reload assembly of claim 9, wherein the center auxiliary pulley is axially coupled to a center auxiliary pulley rotation shaft, and the center auxiliary pulley rotation shaft is arranged to cross an imaginary plane passing through the first driving pulley and the second driving pulley.

11. The reload assembly of claim 10, wherein
the center auxiliary pulley rotation shaft is configured to receive forces from a plurality of wires passing through the center auxiliary pulley, and
a portion of the forces applied in a direction perpendicular to the center auxiliary pulley rotation shaft are partially offset from each other.

12. The reload assembly of claim 10, wherein the first driving pulley and the second driving pulley manipulate a same degree of freedom of the end tool.

13. The reload assembly of claim 9,
wherein the center auxiliary pulley comprises a first auxiliary pulley, a second auxiliary pulley, a third auxiliary pulley, and a fourth auxiliary pulley, all axially coupled about a same rotation shaft,
the first auxiliary pulley and the second auxiliary pulley are arranged on one side with respect to the central axis of the shaft, and
wherein the third auxiliary pulley and the fourth auxiliary pulley are arranged on another side with respect to the central axis of the shaft.

14. The reload assembly of claim 13,
wherein the wire unit comprises a pair of wires including a forward wire and a backward wire,
wherein one of the forward wire and the backward wire passes through one of the first auxiliary pulley and the second auxiliary pulley, and
wherein another of the forward wire and the backward wire passes through one of the third auxiliary pulley and the fourth auxiliary pulley.

15. The reload assembly of claim 8, wherein
the driving pulley unit comprises a first driving pulley,
the auxiliary pulley unit comprises at least one pair of auxiliary pulleys, and
each of the at least one pair of auxiliary pulleys are spaced apart from each other corresponding to a diameter of the first driving pulley around which a pair of wires are wound.

16. The reload assembly of claim 8, wherein the pulley frame is configured to accommodate the driving pulley unit to allow the driving pulley unit to be axially rotatable.

17. The reload assembly of claim 16, wherein the pulley frame comprises:
a first pulley frame comprising an auxiliary pulley fixing part configured to accommodate at least a portion of auxiliary pulley unit; and
a second pulley frame configured to connect the shaft to the first pulley frame.

18. A surgical instrument comprising:
an end tool comprising one or more rotatable jaws and configured to be rotated in at least one direction;
a shaft having an end to which the end tool is connected;
a power generation part configured to generate power for driving the end tool; and
a power transmission part arranged between the shaft and a power generation part and configured to transmit the power generated by the power generation part to the end tool,
wherein the power transmission part comprises:
a pulley frame connected to the power generation part;
a driving pulley unit accommodated in the pulley frame and configured to receive the power generated by the power generation part and perform an axial rotation; and
a wire unit having one end portion connected to the driving pulley unit and another end portion connected to the end tool and configured to transmit the power generated by the power generation part to the end tool,
wherein a rotation shaft of the driving pulley unit is arranged in a direction extending toward the end tool.

19. The surgical instrument of claim 18, further comprising a manipulation part configured to receive, from a user, a signal for controlling an operation of the end tool,
wherein the power generation part comprises:
a motor pack including at least one motor arranged to be at least partially accommodated in a housing of the manipulation part and configured to generate power for driving the end tool based on a signal input to the manipulation part; and
a roll driving motor configured to generate power for a roll rotation of the motor pack.

20. The surgical instrument of claim 19, wherein the power transmission part is connected to the motor pack and configured to be roll-rotated together with the motor pack.
